(19) United States
(12) Patent Application Publication
(10) Pub. No.: US 2012/0295890 A1
(43) Pub. Date: Nov. 22, 2012

(54) GLYCOSAMINOGLYCAN INHIBITORS

(75) Inventors: Brett E. Crawford, Poway, CA (US); Charles A. Glass, San Diego, CA (US); Jillian R. Brown, Poway, CA (US)

(73) Assignee: Zacharon Pharmaceutical, Inc., San Diego, CA (US)

(21) Appl. No.: 13/519,262

(22) PCT Filed: Dec. 28, 2010
(86) PCT No.: PCT/US10/62247

§ 371 Date: Jun. 26, 2012

Related U.S. Application Data
(60) Provisional application No. 61/291,601, filed on Dec. 31, 2009.

(51) Int. Cl. C12P 21/00; C12N 9/10; C12N 5/071; A61K 31/55; G01N 33/53; A61P 25/00; G01N 33/566; C12Q 1/527; A61P 35/00; A61P 29/00; A61P 19/02; A61P 43/00; C12N 9/99; G01N 21/64

(52) U.S. Cl. 514/217.11; 435/69.1; 435/184; 435/193; 435/375; 435/7.1; 435/7.5; 435/29

(57) ABSTRACT

Provided herein are chondroitin sulfate inhibitors, including modulators of glycosylation, and/or sulfation of galactose or N-acetyl galactosamine glycosaminoglycans.

### CROSS-REFERENCE

This application claims the benefit of U.S. Provisional Application No. 61/291,601, filed 31 Dec. 2009, which application is incorporated herein by reference in its entirety.

### STATEMENT AS TO FEDERALLY SPONSORED RESEARCH

Certain inventions described herein were made with the support of the United States government under Grant Applications 1R43NS054350-01A1 and 1R43CA112794-01A1.

### BACKGROUND OF THE INVENTION

Various glycans (e.g., chondroitin sulfate, dermatan sulfate and keratan sulfate) contain galactose or N-acetyl galactosamine containing glycosaminoglycans For example, chondroitin sulfate (CS) is a glycan found in animals comprising galactosamine and glucuronic acid groups. In certain instances, chondroitin sulfate is bound to a core protein via a linkage tetrasaccharide, which generally has the structure -GlcAβ3Galβ3Galβ4XylβO—. FIG. 1 illustrates exemplary structures of chondroitin sulfate, dermatan sulfate and keratan sulfate.

### SUMMARY OF THE INVENTION

Provided in certain embodiments, herein is a process for modifying the structure of a glycan (e.g., chondroitin sulfate), e.g., on a core protein, comprising contacting a cell that translationally produces at least one core protein having at least one attached glycan (e.g., chondroitin sulfate moiety) with a selective inhibitor of a glycan biosynthetic or degradative enzyme, (e.g., a glycosyltransferase, a sulfotransferase, or a phosphotransferase specific for the specific glycan, such as chondroitin sulfate). In some embodiments, the selective inhibitor is an inhibitor of a chondroitin sulfate and/or dermatan sulfate bisynthetic or degradative enzyme or an inhibitor of the production of any oligosaccharide in the chondroitin sulfate and/or dermatan sulfate biosynthetic pathway, such as any enzyme or oligosaccharide described in FIG. 2A, 2B, or 2C. In certain embodiments, the selective inhibitor is a selective inhibitor of a chondroitin sulfate or dermatan sulfate comprising a disaccharide repeat of FIG. 3. In some embodiments, the selective inhibitor is an inhibitor of a keratan sulfate bisynthetic or degradative enzyme or an inhibitor of the production of any oligosaccharide in the keratan sulfate biosynthetic pathway, such as any enzyme or oligosaccharide described in FIG. 4A, or 4B.

For example, in some embodiments, the selective inhibitor of a chondroitin sulfate sulfotransferase is an inhibitor of a chondroitin sulfate O-sulfotransferase. In specific embodiments, the inhibitor of a chondroitin sulfate O-sulfotransferase inhibits the 6-OH sulfation of a galactosaminyl moiety, the 4-OH sulfation of a galactosaminyl moiety, the 2-OH sulfation of a uronic acid moiety, or a combination thereof. In some embodiments, the inhibitor of a chondroitin sulfate glycosyltransferase inhibits the synthesis of the linkage region, the modification of the linkage region, the initiation of chondroitin sulfate synthesis, the synthesis of chondroitin sulfate, or a combination thereof.

Provided in certain embodiments, herein is a process of inhibiting chondroitin sulfate function in a cell comprising contacting the cell with a selective modulator of a chondroitin sulfate glycosyltransferase or a modulator of a chondroitin sulfate sulfotransferase. In certain embodiments, the chondroitin sulfate function inhibited is an ability to bind a chondroitin sulfate binding lectin. In specific embodiments, the chondroitin sulfate lectin is a growth factor. In more specific embodiments, the growth factor is a fibroblast growth factor (FGF), lamin, nuclear ribonucleoprotein, an antibody, Plasmodium falciparum lectin, annexin 4, annexin 6, PTPsigma, endostatin, or any other chondroitin sulfate binding agent. In some embodiments, the modulator of chondroitin sulfate sulfotransferase is an inhibitor of chondroitin sulfate sulfotransferase. In specific embodiments, the inhibitor of chondroitin sulfate sulfotransferase is an inhibitor of chondroitin sulfate O-sulfotransferase. In more specific embodiments, the inhibitor of a chondroitin sulfate O-sulfotransferase inhibits the 6-OH sulfation of a galactosaminyl moiety, the 4-OH sulfation of a galactosaminyl moiety, the 2-OH sulfation of a uronic acid moiety, or a combination thereof. In some embodiments, the modulator of a chondroitin sulfate sulfotransferase is a promoter of the chondroitin sulfate sulfotransferase. In certain embodiments, the modulator of a chondroitin sulfate glycosyltransferase is an inhibitor of the chondroitin sulfate glycosyltransferase. In some embodiments, the modulator of a chondroitin sulfate glycosyltransferase is a promoter of the chondroitin sulfate glycosyltransferase. In certain embodiments, the cell is present in a human diagnosed with cancer.

Provided in some embodiments herein is a process of inhibiting chondroitin sulfate function in a cell comprising contacting the cell with a selective modulator of chondroitin sulfate biosynthesis. In certain embodiments, the selective modulator of chondroitin sulfate biosynthesis inhibits chondroitin glycosylation. In some embodiments, the selective modulator of chondroitin sulfate biosynthesis inhibits sulfation of chondroitin. In certain embodiments, the selective modulator of chondroitin sulfate biosynthesis promotes sulfation of chondroitin. In further or alternative embodiments, the selective modulator of chondroitin sulfate biosynthesis has a molecular weight of less than 1,000 g/mol.

Provided in certain embodiments herein is a method of treating cancer comprising administering a therapeutically effective amount of a selective modulator of chondroitin sulfate glycosylation, or a selective modulator of chondroitin sulfate sulfation. In some embodiments, the selective modulator of chondroitin sulfate biosynthesis inhibits chondroitin glycosylation. In certain embodiments, the selective modulator of chondroitin sulfate promotes chondroitin glycosylation. In some embodiments, the selective modulator of chondroitin sulfate inhibits sulfation of chondroitin. In certain embodiments, the selective modulator of chondroitin sulfate promotes sulfation of chondroitin. In further or alternative embodiments, the selective modulator of chondroitin sulfate biosynthesis has a molecular weight of less than 1,000 g/mol.

Provided in certain embodiments herein is a method of treating a lysosomal storage disease comprising administering a therapeutically effective amount of a selective modulator of chondroitin sulfate glycosylation, or a selective modulator of chondroitin sulfate sulfation. In some embodiments, the lysosomal storage disease is selected from mucopolysaccharidosis. In further or alternative embodiments, the selective modulator of chondroitin sulfate glycosylation is an inhibitor of chondroitin sulfate glycosylation. In further or alternative embodiments, the selective modulator of chondroitin sulfate sulfation is an inhibitor of chondroitin sulfate sulfation.

Provided in certain embodiments herein is a method of treating an inflammatory disease comprising administering a therapeutically effective amount of a selective modulator of chondroitin sulfate glycosylation, or a selective modulator of chondroitin sulfate sulfation. In some embodiments, the inflammatory disease is selected from osteoarthritis. In further or alternative embodiments, the selective modulator of chondroitin sulfate glycosylation is an inhibitor of chondroitin sulfate glycosylation. In further or alternative embodiments, the selective modulator of chondroitin sulfate glycosylation is a promoter of chondroitin sulfate glycosylation. In further or alternative embodiments, the selective modulator of chondroitin sulfate sulfation is an inhibitor of chondroitin sulfate sulfation. In further or alternative embodiments, the selective modulator of chondroitin sulfate sulfation is a promoter of chondroitin sulfate sulfation. In further or alternative embodiments, the selective modulator of chondroitin sulfate biosynthesis has a molecular weight of less than 1,000 g/mol.

Provided in certain embodiments herein is a method of treating injury to the central nervous system (CNS) comprising administering a therapeutically effective amount of a selective modulator of chondroitin sulfate glycosylation, or a selective modulator of chondroitin sulfate sulfation, wherein the modulator promotes axon regeneration. In some embodiments, the selective modulator of chondroitin sulfate glycosylation is an inhibitor of chondroitin sulfate glycosylation. In certain embodiments, the selective modulator of chondroitin sulfate sulfation is an inhibitor of chondroitin sulfate sulfation.

Provided in some embodiments, is a process for identifying a compound that selectively modulates chondroitin sulfate biosynthesis comprising:
a. contacting a mammalian cell with the compound
b. contacting the mammalian cell and compound combination with a first labeled probe and a second labeled probe, wherein the first labeled probe binds chondroitin sulfate and the second labeled probe binds at least one glycan other than chondroitin sulfate;
c. incubating the mammalian cell, compound, the first labeled probe, and the second labeled probe;
d. collecting the first labeled probe that is bound to chondroitin sulfate;
e. collecting the second labeled probe that is bound to at least one glycan other than chondroitin sulfate;
f. detecting or measuring the amount of first labeled probe bound to chondroitin sulfate; and
g. detecting or measuring the amount of the second labeled probe bound to at least one glycan other than chondroitin sulfate.

In some embodiments, the mammalian cell is a human cervical cancer cell (HeLa). In some embodiments the cell is a heparan sulfate deficient cell (for example, CHO-pgsD) that expresses increased levels of sulfated chondroitin sulfate. In certain embodiments, the labeled probe comprises a biotinyl moiety and the process further comprises tagging the labeled probe with streptavidin-Cy5-PE. In some embodiments, the labeled probe comprises a fluorescent label. In certain embodiments, the labeled probe is a labeled growth factor. In some embodiments, the labeled growth factor is labeled fibroblast growth factor 2 (FGF2) lamin, nuclear ribonucleoprotein, an antibody, Plasmodium falciparum lectin, annexin 4, annexin 6, PTPsigma, endostatin, or any other chondroitin sulfate binding agent.

Also provided in some embodiments herein is a process for identifying a compound that modulates chondroitin sulfate biosynthesis comprising:
a. collecting chondroitin sulfate from a first mammalian cell of a selected type, wherein the chondroitin sulfate is sulfated oligosaccharide comprising galactosaminyl groups, and glucuronic acid groups;
b. cleaving the chondroitin sulfate into a plurality of disaccharide component parts;
c. measuring:
   i. the amount of chondroitin sulfate disaccharides produced by the first mammalian cell,
   ii. the amount of 6-OH sulfation of the galactosaminyl groups, the 4-OH sulfation of the galactosaminyl groups, the 2-OH sulfation of the uronic acid groups, or a combination thereof of the chondroitin sulfate,
   iii. the pattern of sulfation; or
   iv. a combination thereof; and
d. contacting and incubating a second mammalian cell of the selected type with the compound;
e. collecting modified chondroitin sulfate from the second mammalian cell, wherein the modified chondroitin sulfate is sulfated oligosaccharide comprising galactosaminyl groups, and glucuronic acid groups;
f. cleaving the modified chondroitin sulfate into a plurality of disaccharide component parts;
g. measuring:
   i. the amount of chondroitin sulfate disaccharides produced by the second mammalian cell,
   ii. the amount of 6-OH sulfation of the galactosaminyl groups, the 4-OH sulfation of the galactosaminyl groups, the 2-OH sulfation of the uronic acid groups, or a combination thereof of the modified chondroitin sulfate,
   iii. the pattern of sulfation; or
   iv. a combination thereof; and
h. comparing:
   i. the amounts of chondroitin sulfate disaccharides produced by the first and second mammalian cells,
   ii. the amounts of 6-OH sulfation of the galactosaminyl groups, the 4-OH sulfation of the galactosaminyl groups, the 2-OH sulfation of the uronic acid groups, pattern of sulfation, or a combination thereof of the chondroitin sulfate and the modified chondroitin sulfate,
   iii. the pattern of sulfation; or a combination thereof.

Further provided in some embodiments herein is a process for modifying the structure of chondroitin sulfate on a core protein, comprising contacting a cell that translationally produces at least one core protein having at least one attached chondroitin sulfate moiety with a selective inhibitor of a chondroitin sulfate glycosyltransferase, a chondroitin sulfate sulfotransferase, or a chondroitin sulfate phosphotransferase.

Also provided in some embodiments herein is a process for modifying the structure of keratan sulfate on a core protein, comprising contacting a cell that translationally produces at least one core protein having at least one attached keratan sulfate moiety with a selective inhibitor of a keratan sulfate glycosyltransferase, a keratan sulfate sulfotransferase, or a keratan sulfate phosphotransferase.

Further provided in some embodiments herein is a process for modifying the structure of dermatan sulfate on a core protein, comprising contacting a cell that translationally produces at least one core protein having at least one attached dermatan sulfate moiety with a selective inhibitor of a dermatan sulfate glycosyltransferase, a dermatan sulfate sulfotransferase, or a dermatan sulfate phosphotransferase.

Provided in some embodiments herein is a process of modulating chondroitin sulfate biosynthesis in a cell comprising contacting the cell with a selective modulator of chondroitin sulfate biosynthesis. In some the embodiments, the selective modulator of chondroitin sulfate biosynthesis alters or disrupts the chain length of chondroitin sulfate compared to endogenous chondroitin sulfate by at least 5%. In certain embodiments, the selective modulator of chondroitin sulfate biosynthesis alters or disrupts the concentration of chondroitin sulfate compared to endogenous chondroitin sulfate by at least 5%. In some embodiments, the selective modulator of chondroitin sulfate biosynthesis alters or disrupts the 2-O sulfation of chondroitin sulfate compared to endogenous chondroitin sulfate by at least 5%. In certain embodiments, the selective modulator of chondroitin sulfate biosynthesis alters or disrupts the 4-O sulfation of chondroitin sulfate compared to endogenous chondroitin sulfate by at least 5%. In some embodiments, the selective modulator of chondroitin sulfate biosynthesis alters or disrupts the 6-O sulfation of chondroitin sulfate compared to endogenous chondroitin sulfate by at least 5%. In certain embodiments, the selective modulator of chondroitin sulfate biosynthesis alters the ratio of 4-O sulfation to 6-O sulfation to below 6.3 to 1. In some embodiments, the selective modulator of chondroitin sulfate biosynthesis alters the ratio of 6-O sulfation to 2-O sulfation to below 0.16 to 1.

Also provided in some embodiments herein is a process of modulating dermatan sulfate biosynthesis in a cell comprising contacting the cell with a selective modulator of dermatan sulfate biosynthesis. In some embodiments, the selective modulator of dermatan sulfate biosynthesis alters the ratio of 2-O sulfation to 4-O sulfation to below 0.13 to 1. In certain embodiments, the selective modulator of dermatan sulfate biosynthesis alters the ratio of 2-O sulfation to 6-O sulfation to below 1.44 to 1. In some embodiments, the selective modulator of dermatan sulfate biosynthesis alters the ratio of 4-O sulfation to 6-O sulfation to below 11.4 to 1. In certain embodiments, the selective modulator of dermatan sulfate biosynthesis alters the ratio of 4-O sulfation to 2-O sulfation to below 7.9 to 1. In some embodiments, the selective modulator of dermatan sulfate biosynthesis alters the ratio of 6-O sulfation to 2-O sulfation to below 0.7 to 1. In certain embodiments, the selective modulator of dermatan sulfate biosynthesis alters the ratio of 6-O sulfation to 4-O sulfation to below 0.09 to 1.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates exemplary structures of chondroitin sulfate, dermatan sulfate and keratan sulfate.

FIGS. 2A, 2B, and 2C illustrate chondroitin/dermatan sulfate biosynthesis and structure.

FIG. 3 illustrates disaccharide units present in chondroitin sulfate.

FIGS. 4A and 4B illustrate keratan sulfate biosynthesis and structure.

FIG. 5 illustrates in the upper diagram chondroitin sulfate inhibition using anti-chondroitin sulfate antibody 2B6 in cells treated with N-(4-chlorophenyl)-2-[(4-methoxyphenyl)amino]-3,5-dinitrobenzamide (2) and in the lower diagram the specificity of inhibition in a lectin panel.

FIG. 6 illustrates in the upper diagram chondroitin sulfate inhibition using anti-chondroitin sulfate antibody 2B6 in cells treated with 4-bromo-N′-[2-(trifluoroacetyl)-1-cyclopenten-1-yl]benzohydrazide (17) and in the lower diagram the specificity of inhibition in a lectin panel.

FIG. 7 illustrates in the upper diagram chondroitin sulfate inhibition using anti-chondroitin sulfate antibody 2B6 in cells treated with 7-[(3-chlorophenyl)(2-pyridinylamino)methyl]-2-methyl-8-quinolinol (59) and in the lower diagram the specificity of inhibition in a lectin panel.

FIG. 8 illustrates in the upper diagram chondroitin sulfate inhibition using anti-chondroitin sulfate antibody 2B6 in cells treated with 7-[(2-fluorophenyl)(2-pyridinylamino)methyl]-2-methyl-8-quinolinol (60) and in the lower diagram the specificity of inhibition in a lectin panel.

FIG. 9 illustrates in the upper diagram chondroitin sulfate inhibition using anti-chondroitin sulfate antibody 2B6 in cells treated with N-{4-[(4-benzyl-1-piperidinyl)carbonyl]-1-phenyl-1H-pyrazol-5-yl}-3-methylbenzamide (98) and in the lower diagram the specificity of inhibition in a lectin panel.

FIG. 10 illustrates in the upper diagram chondroitin sulfate inhibition using anti-chondroitin sulfate antibody 2B6 in cells treated with 7-tert-butyl-2-(trifluoromethyl)-5,6,7,8-tetrahydro[1]benzothieno[2,3-d]pyrimidin-4(3H)-one (99).

FIG. 11 illustrates in the upper diagram chondroitin sulfate inhibition using anti-chondroitin sulfate antibody 2B6 in cells treated with N-[(8-hydroxy-7-quinolinyl)(4-methylphenyl)methyl]cyclohexanecarboxamide (115) and in the lower diagram the specificity of inhibition in a lectin panel.

FIG. 12 illustrates in the upper diagram chondroitin sulfate inhibition using anti-chondroitin sulfate antibody 2B6 in cells treated with N-[(dibenzylamino)carbonothioyl]-2-fluorobenzamide (181) and in the lower diagram the specificity of inhibition in a lectin panel.

FIG. 13 illustrates in the upper diagram chondroitin sulfate inhibition using anti-chondroitin sulfate antibody 2B6 in cells treated with 3-chloro-N-[(dibenzylamino)carbonothioyl]benzamide (185) and in the lower diagram the specificity of inhibition in a lectin panel.

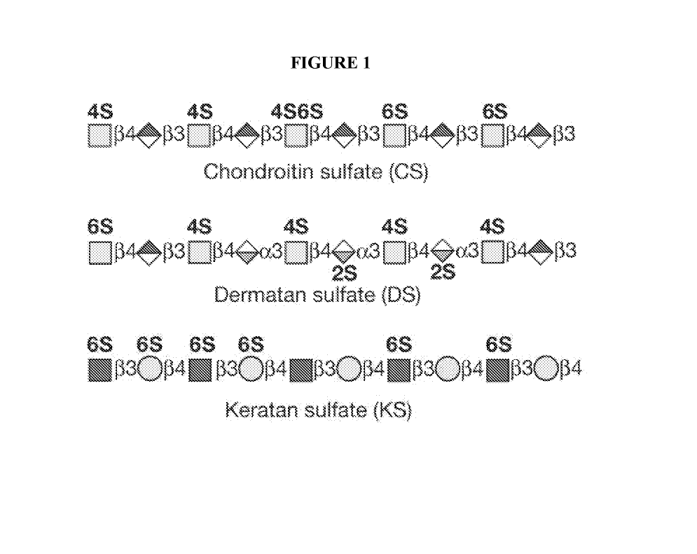

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Glycosaminoglycan Inhibitors

Provided in certain embodiments herein are glycosaminoglycan inhibitors (e.g., galactose or N-acetyl galactosamine containing glycosaminoglycans, such as chondroitin sulfate inhibitors, dermatan sulfate inhibitors, and/or keratan sulfate inhibitors). In general, such inhibitors modulate or alter the nature (e.g., character, structure, or concentration) of the specific glycan or of a class of glycans comprising galactose or N-acetyl galactosamine (e.g., the endogenous glycan on a protein or biomolecule, or in a cell, tissue, organ or individual). Specific description of biosynthesis inhibitors described herein are understood (unless stated otherwise) to include a general disclosure of modulators as well as inhibitors, specifically.

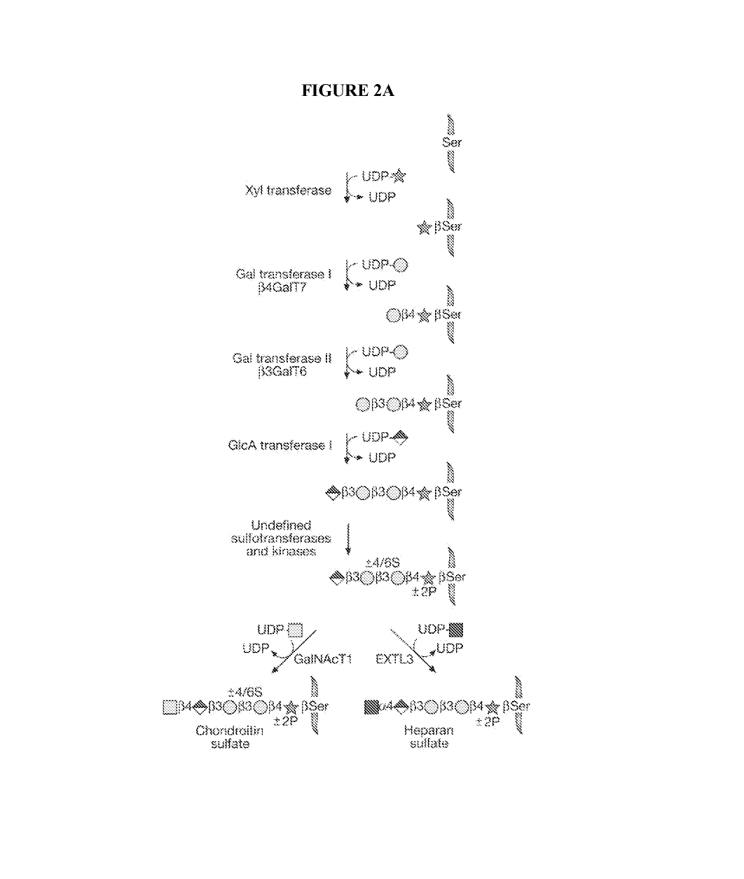

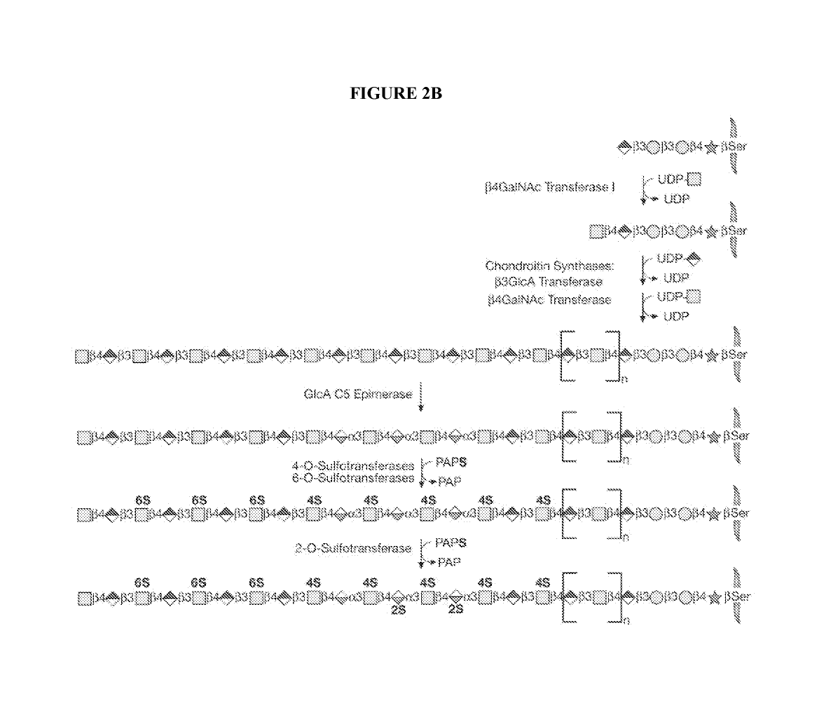

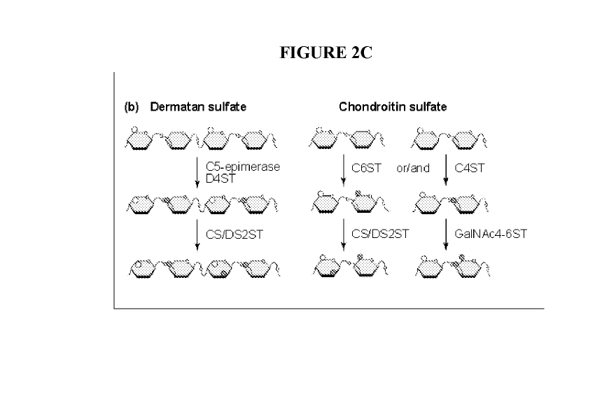

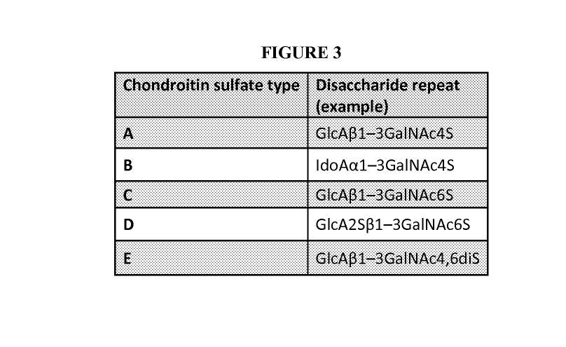

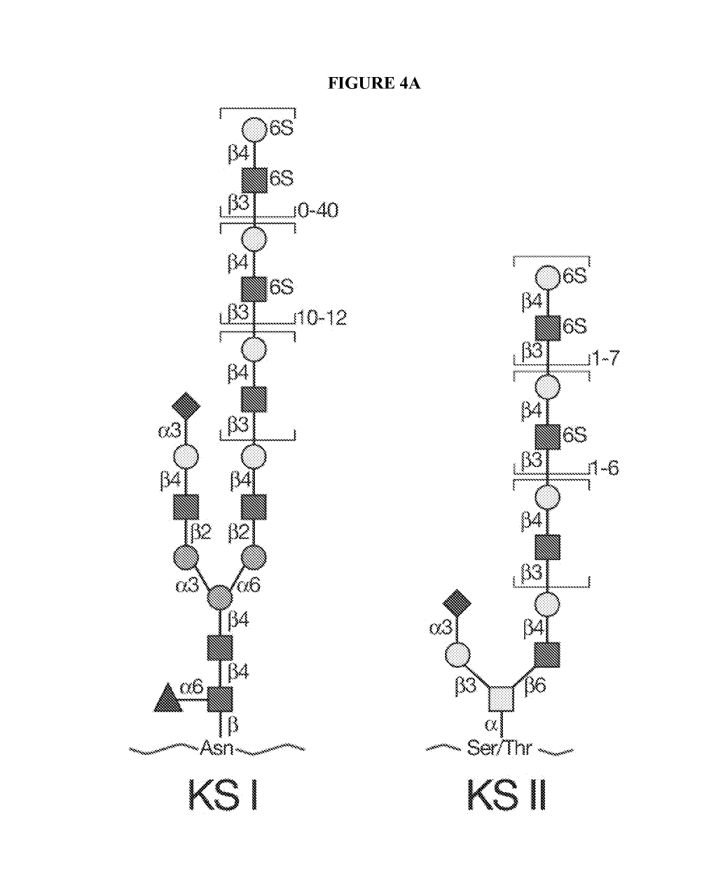

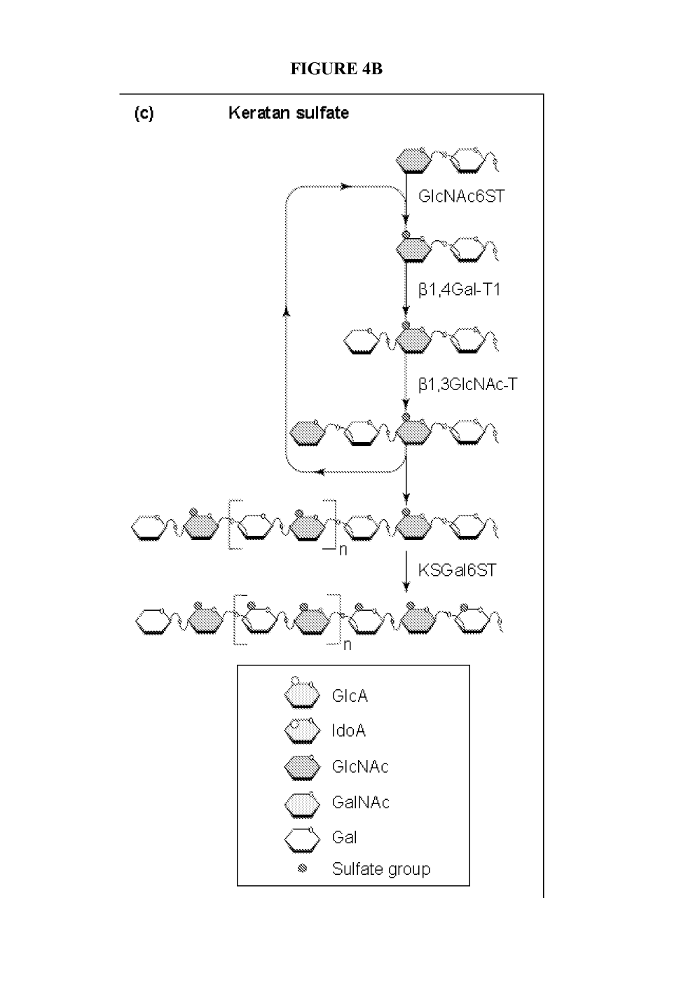

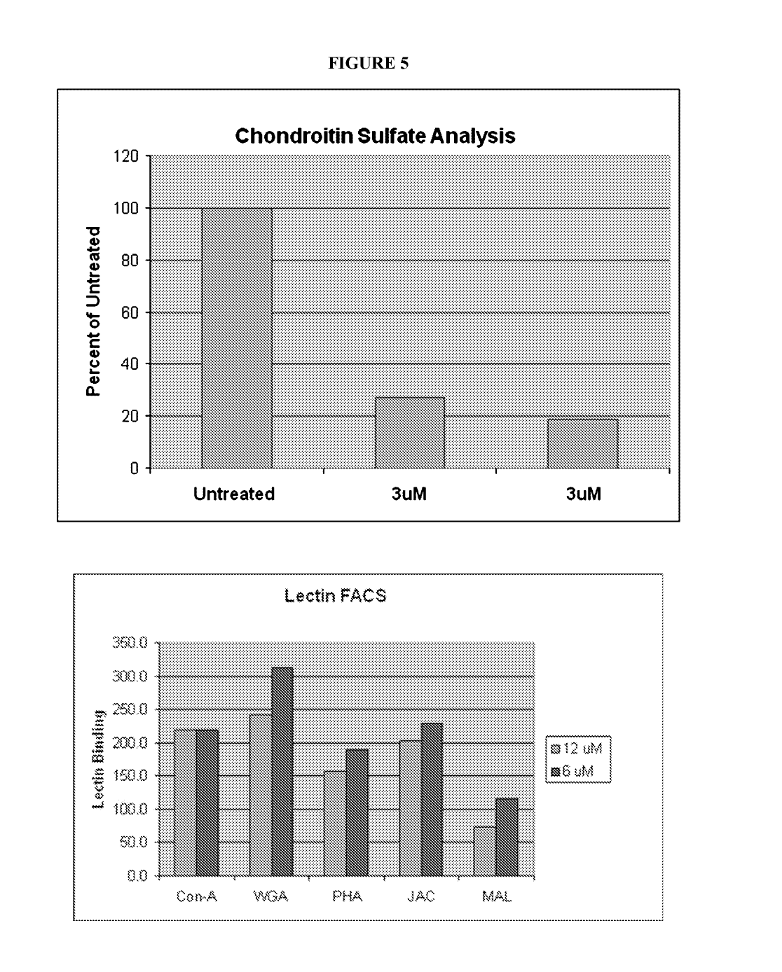

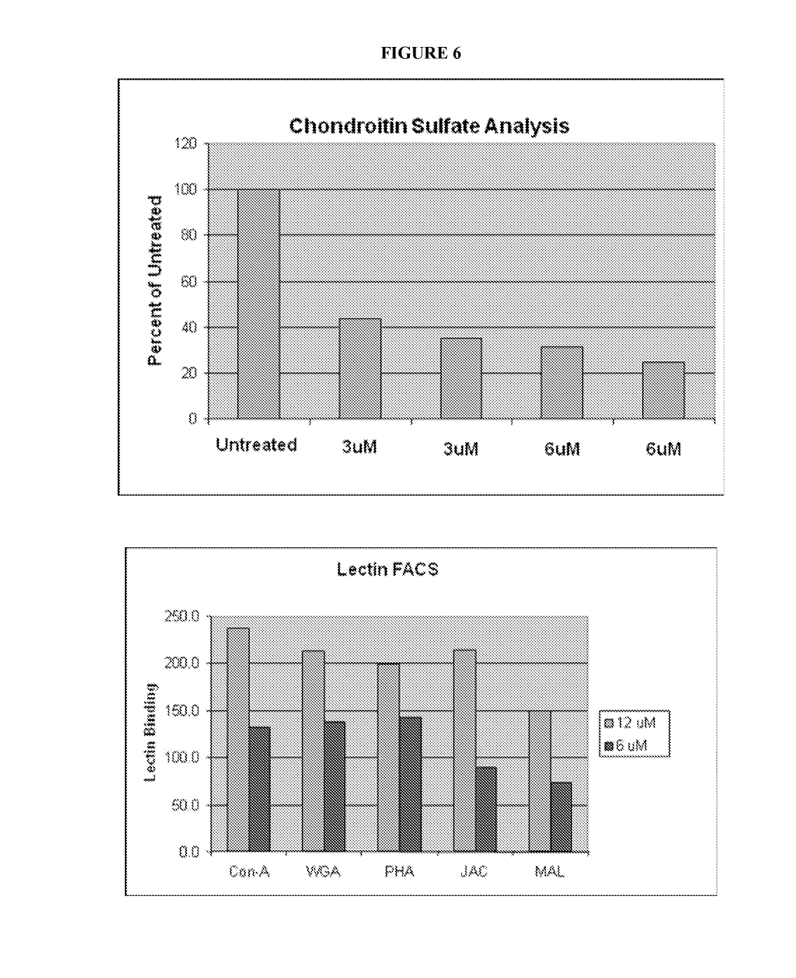

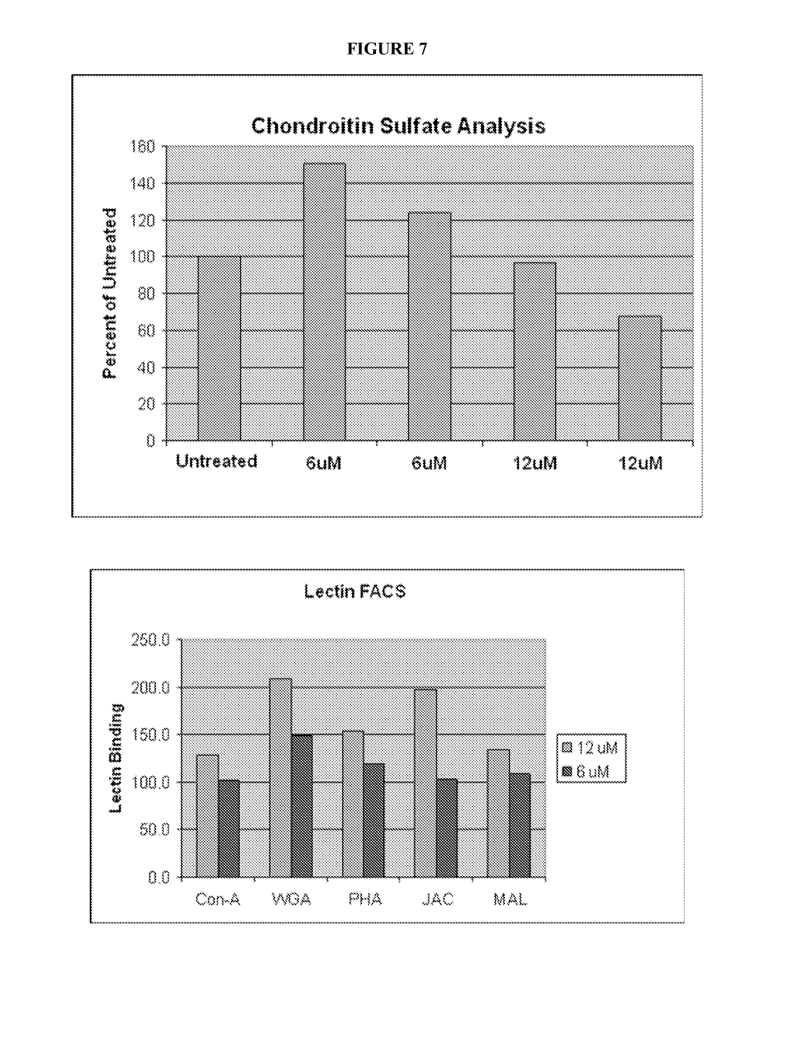

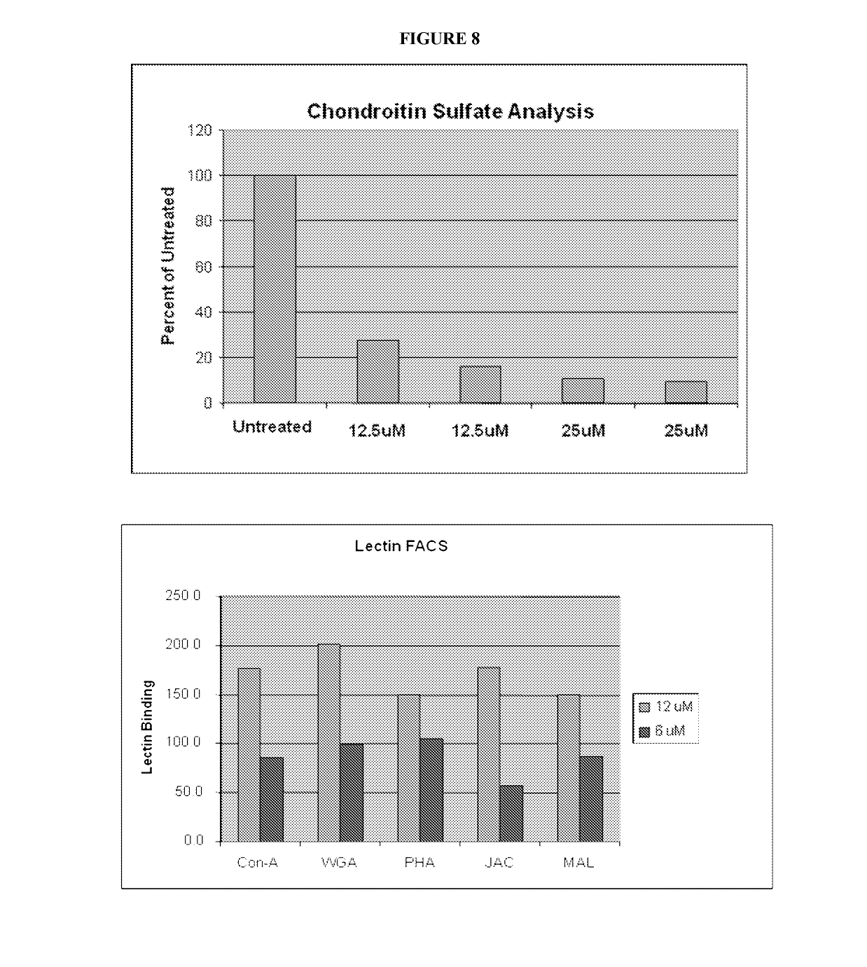

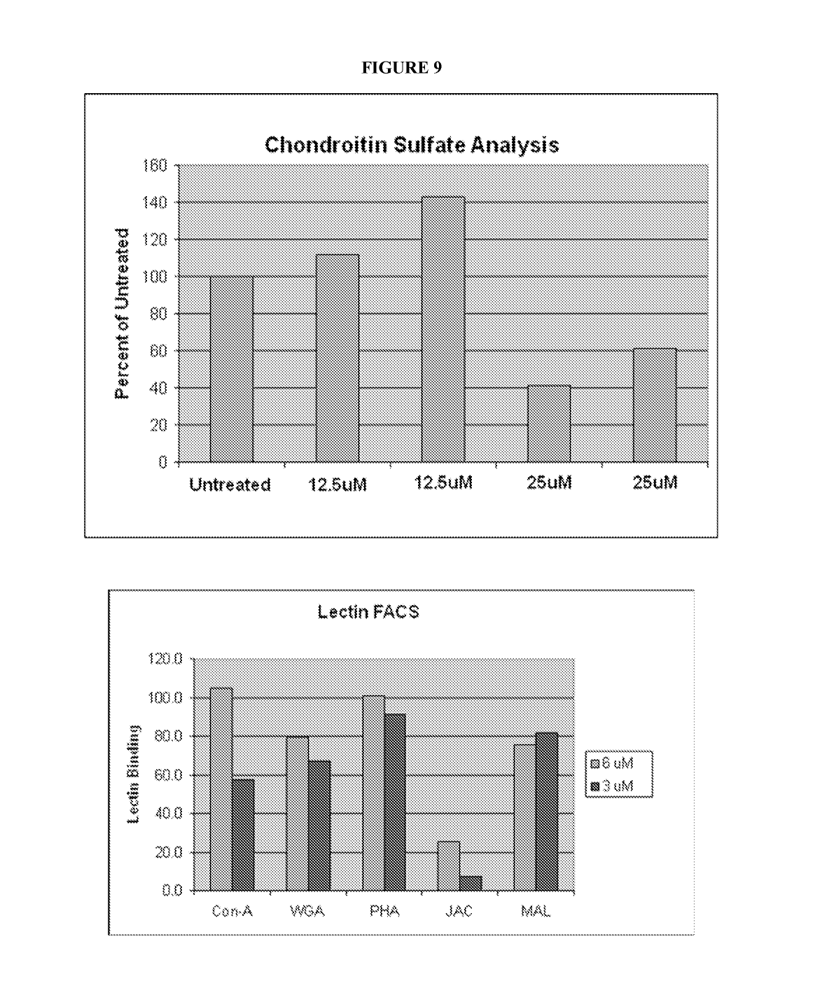

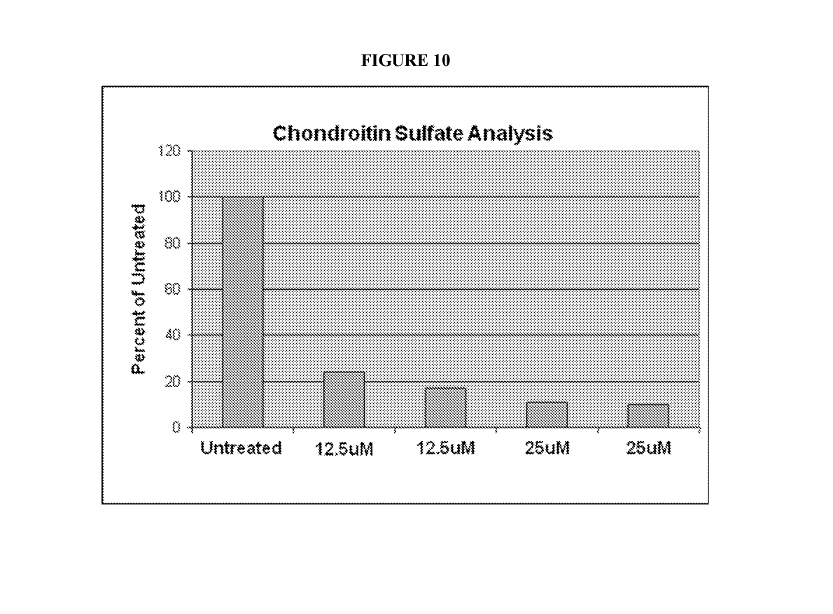

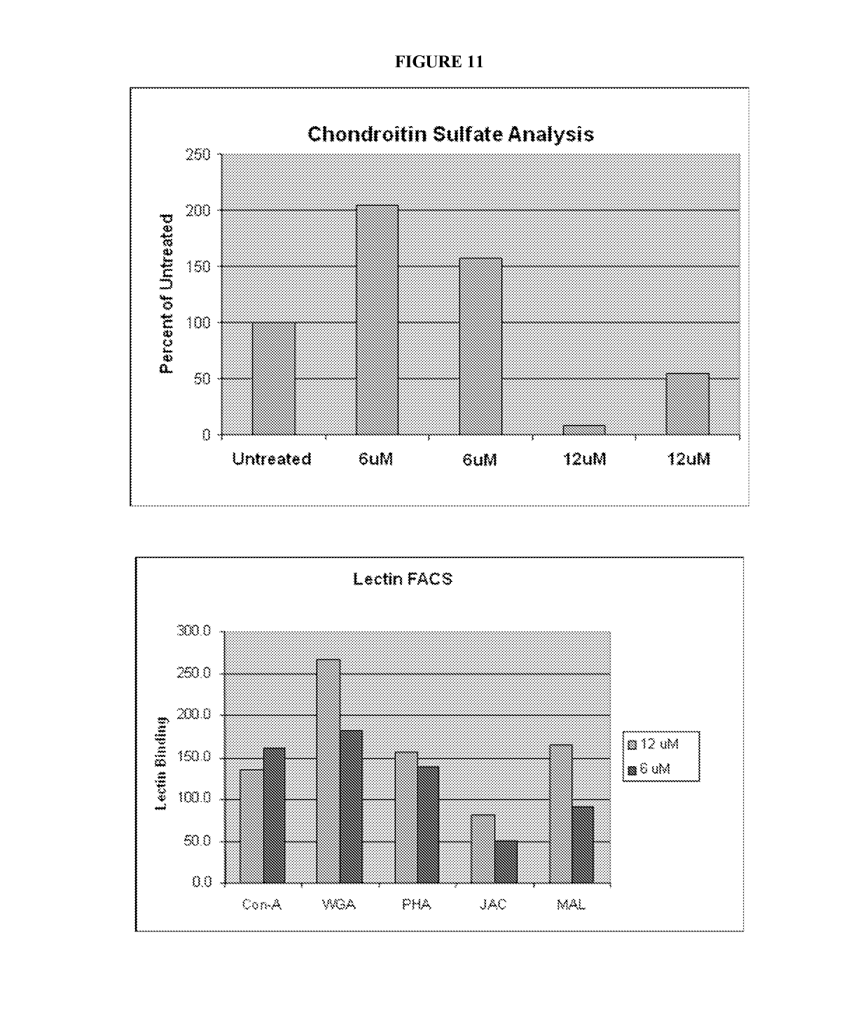

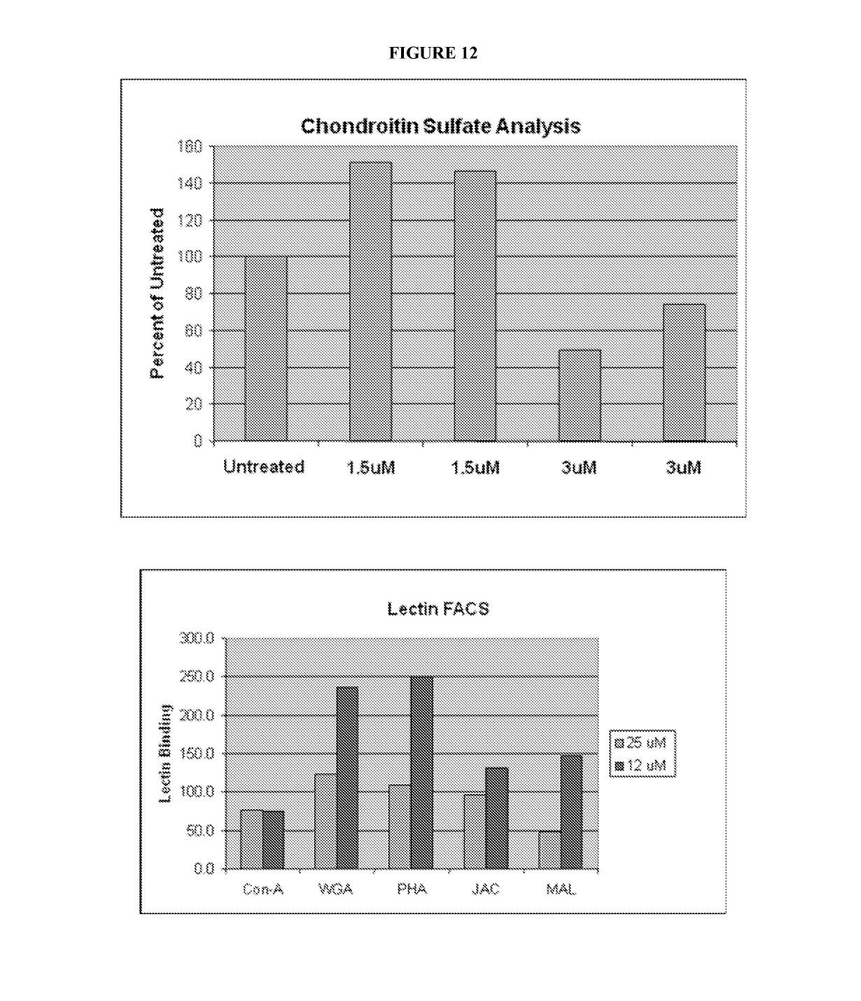

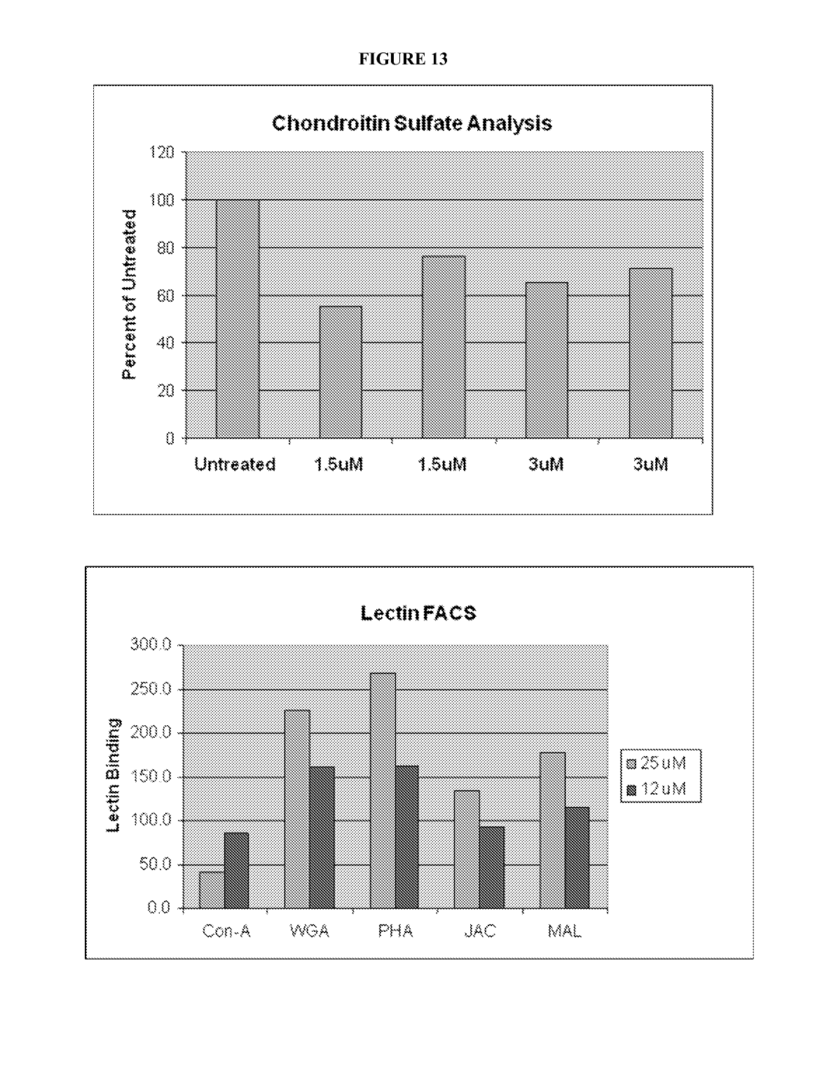

In some embodiments, provided herein is a process for modifying the structure of a glycan (e.g., chondroitin sulfate), e.g., on a core protein, comprising contacting a cell that translationally produces at least one core protein having at least one attached glycan (e.g., chondroitin sulfate moiety) with a selective inhibitor of a glycan biosynthetic or degradative enzyme, (e.g., a glycosyltransferase, a sulfotransferase, or a phosphotransferase specific for the specific glycan, such as chondroitin sulfate). In some embodiments, the selective inhibitor is an inhibitor of a chondroitin sulfate and/or dermatan sulfate bisynthetic or degradative enzyme or an inhibitor of the production of any oligosaccharide in the chondroitin sulfate and/or dermatan sulfate biosynthetic pathway, such as any enzyme or oligosaccharide described in FIG. 2A, 2B, or 2C. In certain embodiments, the selective inhibitor is a selective inhibitor of a chondroitin sulfate or dermatan sulfate comprising a disaccharide repeat of FIG. 3. In some embodiments, the selective inhibitor is an inhibitor of a keratan sulfate bisynthetic or degradative enzyme or an inhibitor of the production of any oligosaccharide in the keratan sulfate biosynthetic pathway, such as any enzyme or oligosaccharide described in FIG. 4A, or 4B.

In specific embodiments, provided herein are methods for identifying small molecule (non-carbohydrate), selective, modulators, of chondroitin sulfate, dermatan sulfate, and/or keratan sulfate biosynthesis, degradation, and/or accumulation by identifying compounds that selectively alter the binding of a lectin that recognizes the target glycan without affecting the binding of lectins that recognize other glycan classes. Provided in further specific embodiments are composition of the glycan produced in the presence of such modulators.

### Galactosamine or Galactose Containing Glycosaminoglycans

Various methods, products, and compositions are described herein. In some instances, methods, products, and compositions are described herein with regard to chondroitin sulfate and/or dermatan sulfate. In various other embodiments, instead of chondroitin sulfate and/or dermatan sulfate, inhibitors described herein instead inhibit dermatan sulfate (e.g., modulate the biosynthesis, degradation, and/or accumulation thereof). In still other embodiments, instead of chondroitin sulfate and/or dermatan sulfate, inhibitors described herein instead inhibit keratan sulfate (e.g., modulate the biosynthesis, degradation, and/or accumulation thereof).

Provided in certain embodiments herein are galactose, galactosamine, glucosamine, or uronic acid containing glycosaminoglycan (e.g., chondroitin sulfate, dermatan sulfate, and/or keratan sulfate) inhibitors, which are also referred to herein simply as glycan inhibitors. In general, glycan inhibitors modulate or alter the nature (e.g., character, structure, or concentration) of one or more glycan (e.g., chondroitin sulfate, dermatan sulfate, and/or keratan sulfate) (e.g., the endogenous glycan on a protein or biomolecule, or in a cell, tissue, organ or individual).

For example, chondroitin sulfate (CS) is a glucuronic acid and N-acetyl galactosamine containing glycosaminoglycan (GAG) comprising a plurality of disaccharide units. One or more of the disaccharide units of chondroitin sulfate comprise an N-acetyl-galactosamine (GalNAc) (Formula I) group linked to a glucuronic acid group (GlcA) (Formula II). Each unit (e.g., N-acetyl-galactosamine or glucuronic acid group) is optionally and independently sulfated. Within the class of compounds described as chondroitin sulfate, there is broad variability with respect to the location and degree of sulfation and other modifications. Therefore, in various instances, glucuronic acid is sometimes O-sulfated at the C2 position (GlcA(2S)); N-acetyl-galactosamine is sometimes O-sulfated at the C6 position (GalNAc(6S)); N-acetyl-galactosamine is sometimes O-sulfated at the C4 position (GalNAc(4S)); N-acetyl-galactosamine is sometimes O-sulfated at the C6 position and the C4 position (GalNAc(4S,6S)); and the like. In certain instances, the disaccharide units are connected to a core protein via and/or comprising a linkage tetrasaccharide, which generally has the structure -GlcAβ3Galβ3Galβ4Xylβ-O—. The linkage tetrasaccharide can be modified by 2-O-phosphorylation of the xylose and 4-O or 6-O sulfation of either of the galactose residues, in any combination. In some instances, the disaccharide units are connected to a core protein at an L-serine amino acid group (e.g., CS-GlcAβ3Galβ3Galβ4Xylβ-O-L-Ser).

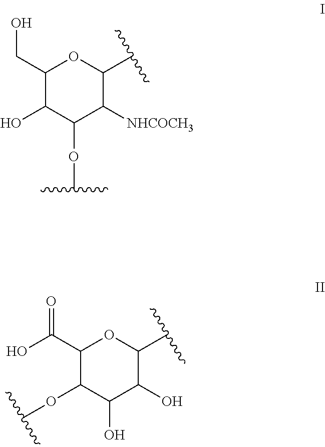

In some embodiments, glycan (e.g., chondroitin sulfate, dermatan sulfate, and/or keratan sulfate) inhibitors described herein modulate glycan (e.g., chondroitin sulfate, dermatan sulfate, and/or keratan sulfate) biosynthesis, e.g., glycan (e.g., chondroitin sulfate, dermatan sulfate, and/or keratan sulfate) glycosylation, glycan (e.g., chondroitin sulfate, dermatan sulfate, and/or keratan sulfate) sulfation, and/or glycan (e.g., chondroitin sulfate, dermatan sulfate, and/or keratan sulfate) phosphorylation. As utilized herein, modulation of glycan (e.g., chondroitin sulfate, dermatan sulfate, and/or keratan sulfate) biosynthesis or the modulation of glycan (e.g., chondroitin sulfate, dermatan sulfate, and/or keratan sulfate) glycosylation or glycan (e.g., chondroitin sulfate, dermatan sulfate, and/or keratan sulfate) sulfation includes the promotion of one or more of and/or the inhibition of one or more of glycan (e.g., chondroitin sulfate, dermatan sulfate, and/or keratan sulfate) glycosylation or glycan (e.g., chondroitin sulfate, dermatan sulfate, and/or keratan sulfate) sulfation.

In some embodiments, the modulation of glycan (e.g., chondroitin sulfate, dermatan sulfate, and/or keratan sulfate) glycosylation includes the modulation of the production of the linkage region that connects glycan (e.g., chondroitin sulfate, dermatan sulfate, and/or keratan sulfate) to a core protein (e.g., GlcAβ3Galβ3Galβ4Xylβ-O—). In certain embodiments, the modulation of the production of the linkage region includes the inhibition of the production of or synthesis of the linkage region. In some embodiments, the modulation of the production of the linkage region includes the cleavage of one or more bonds within the linkage region. In certain instances, a glycan (e.g., chondroitin sulfate, dermatan sulfate, and/or keratan sulfate) inhibitor described herein directly promotes production or cleavage, while in other instances, a glycan (e.g., chondroitin sulfate, dermatan sulfate, and/or keratan sulfate) inhibitor impacts (including modifying the character of) an endogenous chemical (e.g., by activating or deactivating an enzyme) that inhibits production or promotes cleavage of the linkage region. In some embodiments, an inhibitor of glycan (e.g., chondroitin sulfate, dermatan sulfate, and/or keratan sulfate) that modulates the production of the linkage region inhibits one or more glycosyltransferase. In some embodiments, the glycosyltransferase is xylosyltransfarase (e.g., xylosyltransfarase I and/or II), galactosyltransferase (e.g., galactosyltransferase I and/or II), glucuronosyltransferase (e.g., glucuronosyltransferase I), or a combination thereof. In some embodiments, the glycosyltransferase is a uronic acid glycosyl transferase. In more specific embodiments, the glycosyltransferase is a specific uronic acid glycosyl transferase as compared to amino sugar transferases (e.g., GalNAc transferases). In some embodiments, the glycosyltransferase is an amino sugar transferase. In more specific embodiments, the glycosyltransferase is a specific amino sugar transferase as compared to uronic acid glycosyl transferases (e.g., GlcA transferases). In certain instances, specificity includes inhibition of the indicated type of glycosyltransferase by a ratio of greater than 10:1, greater than 9:1, greater than 8:1, greater than 7:1, greater than 6:1, greater than 5:1, greater than 4:1, greater than 3:1, or greater than 2:1 over the other types of glycosyltransferase.

The modulation of glycan (e.g., chondroitin sulfate, dermatan sulfate, and/or keratan sulfate) glycosylation further includes the modulation of the initiation of glycan (e.g., chondroitin sulfate, dermatan sulfate, and/or keratan sulfate) synthesis on the linkage region that connects glycan (e.g., chondroitin sulfate, dermatan sulfate, and/or keratan sulfate) to a core protein (e.g., GlcAβ3Galβ3Galβ4Xylβ-O—). In certain embodiments, the modulation of the initiation of glycan (e.g., chondroitin sulfate, dermatan sulfate, and/or keratan sulfate) synthesis on the linkage region includes the inhibition of the production of or synthesis or modification of the linkage region. In some embodiments, the modulation of the initiation of chondroitin sulfate synthesis to the linkage region includes the cleavage of a bond connecting the first galactosamine group to the linkage region. In certain instances, a glycan (e.g., chondroitin sulfate, dermatan sulfate, and/or keratan sulfate) inhibitor described herein directly promotes synthesis or cleavage, while in other instances, a chondroitin sulfate and/or dermatan sulfate inhibitor impacts an endogenous chemical (e.g., by activating or deactivating an enzyme) that inhibits synthesis or promotes cleavage of a bond connecting the first galactosamine group to the linkage region. In some embodiments, an inhibitor of chondroitin sulfate that modulates the initiation of chondroitin sulfate synthesis to the linkage region inhibits one or more glycosyltransferase, e.g., N-acetylgalactosaminyl transferase (e.g., N-acetylgalactosaminyl transferase I).

The modulation of glycan (e.g., chondroitin sulfate, dermatan sulfate, and/or keratan sulfate) glycosylation further includes the modulation of the synthesis (i.e., polymerization) of glycan. In certain embodiments, the modulation of the synthesis of glycan (e.g., chondroitin sulfate, dermatan sulfate, and/or keratan sulfate) includes the inhibition of synthesis of the glycan and/or cleavage of a glycan sulfate bond. In certain instances, a glycan (e.g., chondroitin sulfate, dermatan sulfate, and/or keratan sulfate) inhibitor described herein directly promotes synthesis or cleavage, while in other instances, a glycan (e.g., chondroitin sulfate, dermatan sulfate, and/or keratan sulfate) inhibitor impacts an endogenous chemical (e.g., by activating or deactivating an enzyme) that inhibits synthesis or promotes cleavage of a glycan bond. In some embodiments, a glycan (e.g., chondroitin sulfate, dermatan sulfate, and/or keratan sulfate) inhibitor that modulates the synthesis of glycan inhibits one or more glycosyltransferase, e.g., glucuronosyltransferase (e.g., glucuronosyltransferase II), N-acetylgalactosaminyl transferase (e.g., N-acetylgalactosaminyl transferase II), or a combination thereof.

The modulation of glycan (e.g., chondroitin sulfate, dermatan sulfate, and/or keratan sulfate) sulfation includes the modulation of the oxygen sulfation (i.e., sulfation of the hydroxy group used interchangeably herein with O-sulfation). In some embodiments, a glycan (e.g., chondroitin sulfate, dermatan sulfate, and/or keratan sulfate) inhibitor modulates one or more sulfotransferase. In some embodiments, modulation of O-sulfation includes the inhibition of the 2-O sulfation of a glucuronic acid group of the chondroitin sulfate and/or dermatan sulfate (used interchangebly herein with a glucuronic acid moiety), the 4-O sulfation of a galactosamine group of the chondroitin sulfate and/or dermatan sulfate (used interchangeably herein with a galactosamine moiety), the 6-O sulfation of a galactosamine group of the chondroitin sulfate and/or dermatan sulfate, or a combination thereof. Furthermore, in some embodiments, modulation of O-sulfation includes the promotion of the 2-O sulfation of a glucuronic acid group of the chondroitin sulfate and/or dermatan sulfate, the 4-O sulfation of a galactosamine group of the chondroitin sulfate and/or dermatan sulfate, the 6-O sulfation of a galactosamine group of the chondroitin sulfate and/or dermatan sulfate, or a combination thereof. In certain instances, a single chondroitin sulfate inhibitor inhibits one type of sulfation while also promoting another. For example, in various embodiments, a single chondroitin sulfate and/or dermatan sulfate inhibitor promotes 6-O sulfation while inhibiting 2-O sulfation, a single chondroitin sulfate and/or dermatan sulfate inhibitor promotes 2-O sulfation while inhibiting 6-O sulfation, a single chondroitin sulfate and/or dermatan sulfate inhibitor promotes 6-O sulfation while inhibiting 4-O sulfation, a single chondroitin sulfate and/or dermatan sulfate inhibitor promotes 6-O and 2-O sulfation while inhibiting 4-O sulfation, or the like. In certain embodiments, the chondroitin sulfate and/or dermatan sulfate inhibitor specifically inhibits, modulates or promotes 2-O sulfation of glucuronic acid groups, the chondroitin sulfate and/or dermatan sulfate inhibitor specifically inhibits, modulates or promotes 4-O sulfation of galactosamine groups, the chondroitin sulfate and/or dermatan sulfate inhibitor specifically inhibits, modulates or promotes 6-O sulfation of galactosamine groups, or a combination thereof. In certain instances, specificity includes inhibition, modulation or promotion of the indicated type of sulfation by a ratio of greater than 10:1, greater than 9:1, greater than 8:1, greater than 7:1, greater than 6:1, greater than 5:1, greater than 4:1, greater than 3:1, or greater than 2:1 over the other types of sulfation.

In certain embodiments, chondroitin sulfate and/or dermatan sulfate inhibitors or modulators of chondroitin sulfate and/or dermatan sulfate biosynthesis are compounds that modify the nature (e.g., character, structure and/or concentration) of chondroitin sulfate and/or dermatan sulfate endogenous to a cellular compartment (including vesicles), cell, tissue, organ or individual when contacted or administered to the cell, tissue, organ or individual. It is to be understood that contacting a cell, tissue, or organ is possible via the administration to an individual within whom such cell, tissue or organ resides. In certain instances, chondroitin sulfate and/or dermatan sulfate inhibitors or modulators of chondroitin sulfate and/or dermatan sulfate biosynthesis modify the character and/or concentration of chondroitin sulfate and/or dermatan sulfate in a targeted type of cell, tissue type or organ. In other instances, chondroitin sulfate and/or dermatan sulfate inhibitors or modulators of chondroitin sulfate and/or dermatan sulfate biosynthesis modify the character and/or concentration of chondroitin sulfate and/or dermatan sulfate in a systemic manner.

In certain embodiments, a glycan (e.g., chondroitin sulfate, dermatan sulfate, and/or keratan sulfate) inhibitor (used interchangeably herein with a modulator glycan (e.g., chondroitin sulfate, dermatan sulfate, and/or keratan sulfate) biosynthesis) alters or disrupts the nature of glycan (e.g., chondroitin sulfate, dermatan sulfate, and/or keratan sulfate) compared to endogenous glycan (e.g., chondroitin sulfate, dermatan sulfate, and/or keratan sulfate) and/or glycan (e.g., chondroitin sulfate, dermatan sulfate, and/or keratan sulfate) in an amount sufficient to alter or disrupt glycan (e.g., chondroitin sulfate, dermatan sulfate, and/or keratan sulfate) binding, glycan (e.g., chondroitin sulfate, dermatan sulfate, and/or keratan sulfate) signaling, or a combination thereof. In some embodiments, the glycan (e.g., chondroitin sulfate, dermatan sulfate, and/or keratan sulfate) inhibitor alters or disrupts the nature of glycan (e.g., chondroitin sulfate, dermatan sulfate, and/or keratan sulfate) in a selected tissue type or organ compared to endogenous glycan (e.g., chondroitin sulfate, dermatan sulfate, and/or keratan sulfate) in the selected tissue type or organ. In some embodiments, the selected tissue is, by way of non-limiting example, brain tissue, connective tissue, liver tissue, kidney tissue, intestinal tissue, skin tissue, or the like. In some embodiments, a glycan (e.g., chondroitin sulfate, dermatan sulfate, and/or keratan sulfate) inhibitor as described herein alters or disrupts the nature of glycan (e.g., chondroitin sulfate, dermatan sulfate, and/or keratan sulfate) compared to endogenous glycan (e.g., chondroitin sulfate, dermatan sulfate, and/or keratan sulfate) by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, or more. In certain embodiments, the glycan (e.g., chondroitin sulfate, dermatan sulfate, and/or keratan sulfate) inhibitor described herein alters or disrupts the concentration of glycan (e.g., chondroitin sulfate, dermatan sulfate, and/or keratan sulfate) compared to endogenous glycan (e.g., chondroitin sulfate, dermatan sulfate, and/or keratan sulfate) in a cell, tissue, organ, or individual by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, or more. In certain embodiments, the glycan (e.g., chondroitin sulfate, dermatan sulfate, and/or keratan sulfate) inhibitor described herein alters or disrupts the sulfation, O-sulfation, the 2-O sulfation, the 4-O sulfation, or the 6-O sulfation of glycan (e.g., chondroitin sulfate, dermatan sulfate, and/or keratan sulfate) compared to endogenous glycan (e.g., chondroitin sulfate, dermatan sulfate, and/or keratan sulfate) in a cell, tissue, organ, or individual by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, or more. In certain embodiments, the glycan (e.g., chondroitin sulfate, dermatan sulfate, and/or keratan sulfate) inhibitor described herein alters or disrupts the chain length (or chondroitin sulfate and/or dermatan sulfate molecular weight) of glycan (e.g., chondroitin sulfate, dermatan sulfate, and/or keratan sulfate) compared to endogenous glycan (e.g., chondroitin sulfate, dermatan sulfate, and/or keratan sulfate) in a cell, tissue, organ, or individual by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, or more. In certain embodiments, the glycan (e.g., chondroitin sulfate, dermatan sulfate, and/or keratan sulfate) inhibitor described herein alters or disrupts, in combination (e.g., the sum of the change in amount of sulfation, concentration, and chain length), the nature of glycan (e.g., chondroitin sulfate, dermatan sulfate, and/or keratan sulfate) compared to endogenous chondr glycan (e.g., chondroitin sulfate, dermatan sulfate, and/or keratan sulfate) in a cell, tissue, organ, or individual by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, or more. In certain embodiments, a glycan (e.g., chondroitin sulfate, dermatan sulfate, and/or keratan sulfate) as described herein alters or disrupts the sulfation and/or phosphorylation of the linkage region of glycan (e.g., chondroitin sulfate, dermatan sulfate, and/or keratan sulfate) compared to endogenous glycan (e.g., chondroitin sulfate, dermatan sulfate, and/or keratan sulfate) in an organism, organ, tissue or cell by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, or more. As used herein, endogenous glycan (e.g., chondroitin sulfate, dermatan sulfate, and/or keratan sulfate) is described as glycan (e.g., chondroitin sulfate, dermatan sulfate, and/or keratan sulfate) present in the absence of treatment or contact with a glycan (e.g., chondroitin sulfate, dermatan sulfate, and/or keratan sulfate) inhibitor. In some embodiments, the comparison between altered or disrupted glycan (e.g., chondroitin sulfate, dermatan sulfate, and/or keratan sulfate) compared to endogenous glycan (e.g., chondroitin sulfate, dermatan sulfate, and/or keratan sulfate) is based on the average characteristic (e.g., the concentration, sulfation, O-sulfation, 2-O sulfation, 4-O sulfation, 6-O sulfation, 2-O phosphorylation, chain length or molecular weight, combinations thereof, or the like) of the altered or disrupted glycan (e.g., chondroitin sulfate, dermatan sulfate, and/or keratan sulfate). Furthermore, in some embodiments, the comparison between altered or disrupted glycan (e.g., chondroitin sulfate, dermatan sulfate, and/or keratan sulfate) is based on a comparison of the sulfated and/or non-sulfated domains of the modified glycan (e.g., chondroitin sulfate, dermatan sulfate, and/or keratan sulfate) to the sulfated and/or non-sulfated domains endogenous glycan (e.g., chondroitin sulfate, dermatan sulfate, and/or keratan sulfate). In some instances, the degree or nature of sulfation in the domains that have high sulfation in endogenous glycan (e.g., chondroitin sulfate, dermatan sulfate, and/or keratan sulfate) are increased or decreased in the modified glycan (e.g., chondroitin sulfate, dermatan sulfate, and/or keratan sulfate). Similarly, in certain instances, the degree or nature of sulfation in the domains that have low sulfation in endogenous glycan (e.g., chondroitin sulfate, dermatan sulfate, and/or keratan sulfate) have increased sulfation in the modified glycan (e.g., chondroitin sulfate, dermatan sulfate, and/or keratan sulfate). The concentration, amount, character, and/or structure of glycan (e.g., chondroitin sulfate, dermatan sulfate, and/or keratan sulfate) can be determined in any suitable manner, including those set forth herein. As used herein, altering includes increasing or decreasing. Furthermore, as used herein, disrupting includes reducing or inhibiting.

In specific embodiments, the glycan (e.g., chondroitin sulfate, dermatan sulfate, and/or keratan sulfate) inhibitor described herein alters or disrupts the nature of the glycan (e.g., chondroitin sulfate, dermatan sulfate, and/or keratan sulfate) such that it inhibits glycan (e.g., chondroitin sulfate, dermatan sulfate, and/or keratan sulfate) signaling. In other specific embodiments, the glycan (e.g., chondroitin sulfate, dermatan sulfate, and/or keratan sulfate) inhibitor described herein alters or disrupts the nature of the glycan (e.g., chondroitin sulfate, dermatan sulfate, and/or keratan sulfate) such that it inhibits glycan (e.g., chondroitin sulfate, dermatan sulfate, and/or keratan sulfate) binding. In more specific embodiments, the glycan (e.g., chondroitin sulfate, dermatan sulfate, and/or keratan sulfate) inhibitor described herein alters or disrupts the nature of the glycan (e.g., chondroitin sulfate, dermatan sulfate, and/or keratan sulfate) such that it inhibits glycan (e.g., chondroitin sulfate, dermatan sulfate, and/or keratan sulfate) binding and glycan (e.g., chondroitin sulfate, dermatan sulfate, and/or keratan sulfate) signaling. In some embodiments, the glycan (e.g., chondroitin sulfate, dermatan sulfate, and/or keratan sulfate) inhibitor alters or disrupts the nature of the glycan (e.g., chondroitin sulfate, dermatan sulfate, and/or keratan sulfate) such that it inhibits the binding, signaling, or a combination thereof of any lectin (including polypeptides) subject to glycan (e.g., chondroitin sulfate, dermatan sulfate, and/or keratan sulfate) binding, signaling or a combination thereof, in the absence of a glycan (e.g., chondroitin sulfate, dermatan sulfate, and/or keratan sulfate) inhibitor. In some embodiments, the polypeptide is, by way of non-limiting example, a growth factor. In specific embodiments, the growth factor is, by way of non-limiting example, pleiotrophin, midkine, vascular endothelial growth factor (VEGF), fibroblast growth factor (FGF), hepatocyte growth factor, heparin co-factor II or heparin-binding epidermal growth factor (HB-EGF).

In specific embodiments, selective inhibitors of glycosaminoglycans comprising galactose or N-acetyl galactosamine, following contacting a cell with such inhibitors:
a Inhibit binding of one or more or all of the lectins and antibodies to glycans produced by the cell:
   i. 2B6, MO-225, LY111, NC21C, 1-B-5,2-B-6,3-B-3, Factor H, HC-II (e.g., demonstrates inhibition of CS/DS)
   ii. 5-D-4, galectins (e.g., demonstrates inhibition of KS)
b. But not one or more or all of:
   i. WGA, MAL (e.g., demonstrates inhibitors that selectively inhibit glycosaminoglycans containing galactose or N-acetyl galactosamine over O-linked glycans)
   ii. PHA, ConA (e.g., demonstrates inhibitors that selectively inhibit glycosaminoglycans containing galactose or N-acetyl galactosamine over N-linked glycans)
   iii. FGF2 (e.g., demonstrates inhibitors that selectively inhibit glycosaminoglycans containing galactose or N-acetyl galactosamine over heparan sulfate)
   iv. CTB (cholera toxin B-subunit) (e.g., demonstrates inhibitors that selectively inhibit glycosaminoglycans containing galactose or N-acetyl galactosamine over gangliosides)

In some embodiments, a glycan (e.g., chondroitin sulfate, dermatan sulfate, and/or keratan sulfate) inhibitor described herein is a selective chondroitin sulfate, dermatan sulfate, or keratan sulfate inhibitor. In some embodiments, the selective inhibitor selective alters or disrupts the nature (e.g., concentration, chain length, sulfation, etc.) of chondroitin sulfate, dermatan sulfate, and/or keratan sulfate compared to other glycans. In certain embodiments, the selective inhibitor selectively affects the biosynthesis of glycosaminoglycans (GAGs), such as chondroitin sulfate, dermatan sulfate, keratan sulfate, and/or hyaluronan, but not other glycans (e.g., N-linked, O-linked, lipid linked, or the like). In certain embodiments, selective chondroitin sulfate inhibitors selectively inhibit GAGs compared to other glycans or non-GAG glycans by a ratio of greater than 2:1, 3:1, 4:1, 5:1, 6:1, 8:1, 10:1 or more. In some embodiments, the selective inhibitor selectively affects the biosynthesis of sulfated GAGs, but not non-sulfated GAGs (e.g., hyaluronan) or other non-GAG glycans (e.g., N-linked glycans, O-linked glycans, gangliosides, etc.). In certain embodiments, selective inhibitors selectively inhibit sulfated GAGs compared to non-sulfated GAGs and non-GAG glycans by a ratio of greater than 2:1, 3:1, 4:1, 5:1, 6:1, 8:1, 10:1 or more. In some embodiments, selective inhibitors selectively inhibit the biosynthesis of chondroitin sulfate, chondroitin sulfate and dermatan sulfate, but not keratan sulfate, non-sulfated GAGs or non-GAG glycans. In certain embodiments, selective inhibitors selectively inhibit chondroitin sulfate, chondroitin sulfate and dermatan sulfate, compared to keratan sulfate, non-sulfated GAGs and non-GAG glycans by a ratio of greater than 2:1, 3:1, 4:1, 5:1, 6:1, 8:1, 10:1 or more. In some embodiments, selective inhibitors selective inhibit chondroitin sulfate, but not other glycans (e.g., other GAGs and non-GAG glycans). In certain embodiments, selective inhibitors selectively inhibit chondroitin sulfate compared to other GAGs and non-GAG glycans by a ratio of greater than 2:1, 3:1, 4:1, 5:1, 6:1, 8:1, 10:1 or more.

In specific embodiments, selective glycan inhibitors described herein selectively inhibit one or more of 2.8.2.B1 APS sulfotransferase, 2.8.2.1 aryl sulfotransferase, 2.8.2.5 chondroitin 4-sulfotransferase, 2.8.2.7 UDP-N-acetylgalactosamine-4-sulfate sulfotransferase, 2.8.2.8 [heparan sulfate]-glucosamine N-sulfotransferase, 2.8.2.11 galactosylceramide sulfotransferase, 2.8.2.12 heparitin sulfotransferase, 2.8.2.17 chondroitin 6-sulfotransferase, 2.8.2.21 keratan sulfotransferase, 2.8.2.23 [heparan sulfate]-glucosamine 3-sulfotransferase 1, 2.8.2.24 desulfoglucosinolate sulfotransferase, 2.8.2.29 [heparan sulfate]-glucosamine 3-sulfotransferase 2, 2.8.2.30 [heparan sulfate]-glucosamine 3-sulfotransferase 3, 2.8.2.33 N-acetylgalactosamine 4-sulfate 6-O-sulfotransferase.

Furthermore, in certain embodiments, glycan (e.g., chondroitin sulfate, dermatan sulfate, and/or keratan sulfate) inhibitors selectively modulate specific types of action that inhibit galactose or N-acetyl galactosamine glycan (e.g., chondroitin sulfate, dermatan sulfate, and/or keratan sulfate) function. For example, in some embodiments, glycan (e.g., chondroitin sulfate, dermatan sulfate, and/or keratan sulfate) inhibitors selectively modulate sulfation, glycosylsation, or phosphorylation.

In some embodiments, certain glycan (e.g., chondroitin sulfate, dermatan sulfate, and/or keratan sulfate) inhibitors selectively modulate (e.g., promote or inhibit) 2-O sulfation over other types of sulfation (e.g., 6-O, or 4-O). In some embodiments, certain glycan (e.g., chondroitin sulfate, dermatan sulfate, and/or keratan sulfate) inhibitors selectively modulate (e.g., promote or inhibit) 6-O sulfation. In some embodiments, certain glycan (e.g., chondroitin sulfate, dermatan sulfate, and/or keratan sulfate) inhibitors selectively modulate (e.g., promote or inhibit) 4-sulfation. In some embodiments, certain glycan (e.g., chondroitin sulfate, dermatan sulfate, and/or keratan sulfate) inhibitors selectively modulate (e.g., promote or inhibit) 2-O phosphorylation.

In some embodiments, certain glycan (e.g., chondroitin sulfate, dermatan sulfate, and/or keratan sulfate) inhibitors selectively modulate (e.g., promote or inhibit) glycosyltransferase, and/or specific types of glycosyltransferase. In some embodiments, glycan (e.g., chondroitin sulfate, dermatan sulfate, and/or keratan sulfate) inhibitors selectively modulate (e.g., promote or inhibit) one of a xylosyltransfarase, a galactosyltransferase, a glucuronosyltransferase, or an N-acetylgalactosaminyl transferase. In more specific embodiments, glycan (e.g., chondroitin sulfate, dermatan sulfate, and/or keratan sulfate) inhibitors selectively modulate (e.g., promote or inhibit) one of xylosyltransfarase I, xylosyltransfarase II, galactosyltransferase I, galactosyltransferase II, glucuronosyltransferase I, glucuronosyltransferase II, N-acetylgalactosaminyl transferase I, or N-acetylgalactosaminyl transferase II.

In certain embodiments, glycan (e.g., chondroitin sulfate, dermatan sulfate, and/or keratan sulfate) sulfate inhibitors described herein are small molecule organic compounds. Thus, in certain instances, glycan (e.g., chondroitin sulfate, dermatan sulfate, and/or keratan sulfate) inhibitors utilized herein are not polypeptides or carbohydrates. In some embodiments, in certain embodiments, a small molecule organic compound has a molecular weight of less than 2,000 g/mol, less than 1,500 g/mol, less than 1,000 g/mol, less than 700 g/mol, or less than 500 g/mol.

In some embodiments, provided herein are glycan modulators that alter the normal human serum levels of chondroitin sulfate and/or dermatan sulfate. Normal human serum chondroitin sulfate and dermatan sulfate are summarized in Tables 1-3.

TABLE 1

| % Composition | OS | 4S | 2S | 6S | 2S4S | 4S6S | 2S6S |
|---|---|---|---|---|---|---|---|
| Dermatan | 0.5 | 80.5 | 3.0 | 4.5 | 8.0 | 3.0 | 0.5 |
| Sulfate | | | | | | | |
| Chondroitin | 52.5 | 41.0 | 0.0 | 6.5 | 0.0 | 0.0 | 0.0 |
| Sulfate | | | | | | | |

In specific embodiments, inhibitors described herein alter the CS or DS serum compositions to reduce mono 4-sulfated disaccharides to below 80.5%, below 75%, below 70%, below 65%, below 60%, below 55%, below 50%, below 40%, below 30%, below 20%, below 10%, between 10% and 80%, between 10% and 70%, between 20% and 60%, between 20% and 70%, or the like in DS or below 41%, below 40%, below 30%, below 20%, below 10%, between 10% and 40%, between 10% and 30%, between 20% and 30%, between 20% and 40%, or the like in CS; reduce mono 2-sulfated disaccharides to less than 3%, less than 2%, less than 1%, less than 0.5%, between 0.1% and 3%, between 0.1% and 2%, or the like in DS, or mono 6-sulfated disaccharides to less than 4.5%, less than 4%, less than 3%, less than 2%, less than 1%, less than 0.5%, between 0.1% and 4%, between 0.1% and 3%, or the like in DS or less than 6.5%, less than 6%, less than 5%, less than 4%, less than 3%, less than 2%, less than 1%, less than 0.5%, between 0.1% and 3%, between 0.1% and 2%, or the like in CS; reduce disulfated 2S4S disaccharides to less than 8%, less than 7%, less than 6%, less than 5% less than 4%, less than 3%, less than 2%, less than 1%, less than 0.5%, between 0.1% and 8%, between 0.1% and 6%, or the like in DS; reduce disulfated 4S6S disaccharides to less than 3%, less than 2%, less than 1%, less than 0.5%, between 0.1% and 3%, between 0.1% and 2%, or the like in DS; reduce disulfated 2S6S disaccharides to less than 0.5%, less than 0.4%, less than 0.3%, less than 0.2%, less than 0.1%, between 0.01% and 0.5%, between 0.01% and 0.4%, or the like in DS. Alternatively in some embodiments, an inhibitor increases nonsulfated disaccharides to greater than 52.5%, greater than 55%, greater than 60%, greater than 65%, greater than 70%, greater than 80%, greater than 90%, 55-95%, 60-90%, or the like in CS or >0.5%, >1%, >2%, >3%, >5%, >10%, 0.5-90%, 1-50%, 2-50%, or the like in DS. Also provided in certain embodiments, are compositions comprising human serum and a chondroitin sulfate and/or dermatan sulfate as described above, and optionally further comprising any inhibitor described herein.

TABLE 2

| % Composition | 4S | 2S | 6S |
|---|---|---|---|
| Dermatan | 91.5 | 11.5 | 8.0 |
| Sulfate | | | |
| Chondroitin | 41.0 | 0.0 | 6.5 |
| Sulfate | | | |

In some embodiments, inhibitors described herein reduce the 4-sulfation to less than 91.5%, less than 90%, less than 95%, less than 90%, less than 85%, less than 80%, less than 70%, less than 60%, less than 50%, 10-90%, 5-90%, 1-90%, 10-80%, or the like of DS or less than 41%, below 40%, below 30%, below 20%, below 10%, between 10% and 40%, between 10% and 30%, between 20% and 30%, between 20% and 40%, or the like in CS, reduce 2-O sulfation to less than 11.5%, less than 11%, less than 10%, less than 9%, less than 8%, less than 7%, less than 6%, less than 5% less than 4%, less than 3%, less than 2%, less than 1%, less than 0.5%, between 0.1% and 11%, between 0.1% and 10%, or the like in DS, or reduce 6-O sulfation to less than 8.0%, less than 7%, less than 6%, less than 5% less than 4%, less than 3%, less than 2%, less than 1%, less than 0.5%, between 0.1% and 8%, between 0.1% and 6%, or the like in DS or less than 6.5%, less than 6%, less than 5% less than 4%, less than 3%, less than 2%, less than 1%, less than 0.5%, between 0.1% and 6%, between 0.1% and 5%, or the like in CS. Also provided in certain embodiments, are compositions comprising human serum and a chondroitin sulfate and/or dermatan sulfate as described above, and optionally further comprising any inhibitor described herein.

TABLE 3

| % Composition | 2S:4S | 2S:6S | 4S:6S | 4S:2S | 6S:2S | 6S:4S |
|---|---|---|---|---|---|---|
| Dermatan | 0.13 | 1.44 | 11.44 | 7.96 | 0.70 | 0.09 |
| Sulfate | | | | | | |
| Chondroitin | 0.00 | 0.00 | 6.31 | — | — | 0.16 |
| Sulfate | | | | | | |

In some embodiments, inhibitors alter the ratio of sulfation types, for example a 2-O sulfation inhibitor would alter the ratio of 2-O sulfation to 4-O sulfation to below 0.13 to 1, below 0.12:1, below 0.1:1, below 0.9:1, below 0.5:1, between 0.01:1 and 0.13:1, between, 0.01:1 and 0.1:1, or the like or the ratio of 2-O sulfation to 6-O sulfation to below 1.44 to 1, below 0.13 to 1, below 0.12:1, below 0.1:1, below 0.9:1, below 0.5:1, between 0.01:1 and 0.13:1, between, 0.01:1 and 0.1:1, or the like. A 4-O sulfation inhibitor would alter the ratio of 4-O sulfation to 6-O sulfation to below 11.4 to 1, below 11 to 1, below 10:1, below 9:1, below 5:1, below 2:1, between 0.01:1 and 11:1, between, 0.01:1 and 10:1, or the like or the ratio of 4-O sulfation to 2-O sulfation to below 7.9 to 1, below 7.5 to 1, below 7:1, below 6:1, below 5:1, below 2:1, between 0.01:1 and 7.5:1, between, 0.01:1 and 7:1, or the like in DS or the ratio of 4-O sulfation to 6-O sulfation to below 6.3 to 1, below 6 to 1, below 5:1, below 4:1, below 3:1, below 2:1, between 0.01:1 and 6:1, between, 0.01:1 and 3:1, or the like in CS. A 6-O sulfation inhibitor would alter the ratio of 6-O sulfation to 2-O sulfation to below 0.7 to 1, below 0.6 to 1, below 0.5:1, below 0.3:1, below 0.2:1, between 0.01:1 and 0.65:1, between, 0.01:1 and 0.6:1, or the like or the ratio of 6-O sulfation to 4-O sulfation to below 0.09 to 1, below 0.08 to 1, below 0.07 to 1, below 0.05:1, below 0.03:1, below 0.02:1, between 0.001:1 and 0.065:1, between, 0.01:1 and 0.05:1, or the like in DS or the ratio of 6-O sulfation to 4-O sulfation to below 0.16 to 1, below 0.15 to 1, below 0.12 to 1, below 0.1:1, below 0.05:1, below 0.01:1, between 0.001:1 and 0.15:1, or the like in CS. Also provided in certain embodiments, are compositions comprising human serum and a chondroitin sulfate and/or dermatan sulfate as described above, and optionally further comprising any inhibitor described herein.

In some embodiments, provided herein are glycan modulators that alter the normal bovine levels of keratan sulfate. Normal bovine chondrocytes (connective tissue) keratan sulfate is summarized in Tables 4-6.

TABLE 4

| KS Disaccharide | pg/cell | % |
|---|---|---|
| galβ1,4glcNAc6S | 0.0015 | 12 |
| gal6Sβ1,4glcNAc6S | 0.0025 | 20 |
| glcNAcβ1,3gal | 0.0025 | 20 |
| glcNAc6Sβ1,3gal | 0.006 | 48 |

In some embodiments, inhibitors alter the composition of KS to reduce the abundance of 6-sulfated saccharides. For example, in some embodiments, inhibitors reduce the abundance of keratanase and endogalactosidase released disaccharides such as galβ1,4glcNAc6S to below 12%, below 10%, below 8%, below 7%, below 5%, between 0.1% and 11%, between 1% and 10%, or the like, or gal6Sβ1,4glcNAc6S to below 20%, below 19%, below 18%, below 15%, below 10%, below 5%, 1-19%, 0.1-19%, 0.1-15%, or the like, glcNAcβ1,3gal to below 20%, below 19%, below 18%, below 15%, below 10%, below 5%, 1-19%, 0.1-19%, 0.1-15%, or the like, or glcNAc6Sβ1,3gal to below 48%, below 47%, below 45%, below 40%, below 30%, below 25%, below 10%, below 5%, 1-45%, 0.1-45%, 0.1-25%, or the like. Also provided in certain embodiments, are bovine compositions comprising a keratan sulfate as described above, and optionally further comprising any inhibitor described herein.

TABLE 5

| Sulfation type | % |
|---|---|
| 6S-GlcNAc | 80 |
| 6S-Gal | 20 |

In some embodiments, inhibitors alter the extent of 6-O sulfation to below 80%, below 78%, below 75%, below 70%, below 50%, below 25%, below 10%, below 5%, 1-75%, 0.1-75%, 0.1-50%, or the like of the GlcNAc residues and/or less than 20%, below 19%, below 18%, below 15%, below 12%, below 10%, below 5%, 1-19%, 0.1-19%, 0.1-15%, or the like of the galactose residues that are liberated into disaccharides by the treatment with keratanase and endogalactosidase. Also provided in certain embodiments, are bovine compositions comprising a keratan sulfate as described above, and optionally further comprising any inhibitor described herein.

TABLE 6

| | 6SGlcNAc:6SGal | 6SGal:6SGlcNAc |
|---|---|---|
| Ratio | 4 | .25 |

In some embodiments, inhibitors of GlcNAc 6-O sulfation alter the ratio of 6-O sulfation of GlcNAc to Galactose to less than 4:1, less than 3.5:1, less than 3:1, less than 2:1, less than 1.5:1, 0.1:1 to 4:1, 0.1:1 to 3.9:1, 0.1:1 to 3:1, or the like and desirable inhibitors of galactose 6-O sulfation would alter the ratio of 6-O sulfation of GlcNAc to Galactose to greater than 4:1, less than 3.5:1, less than 3:1, less than 2:1, less than 1.5:1, 0.1:1 to 4:1, 0.1:1 to 3.9:1, 0.1:1 to 3:1, or the like. Also provided in certain embodiments, are bovine compositions comprising a keratan sulfate as described above, and optionally further comprising any inhibitor described herein.

As discussed throughout this specification, in some embodiments, provided herein is a glycan inhibitor that is a non-carbohydrate—small molecule. In certain instances, carbohydrates tend to be hydrophilic due to the polyhydroxyls and therefore do not diffuse into cells efficiently. Carbohydrates typically have pharmacokinetic and pharmacodynamic properties in animals that are inappropriate for therapeutic drug effects. Further, in some instances, the hydroxyls are reactive and make carbohydrates difficult and expensive to synthesize. The range of possible structures is limited compared to noncarbohydrate small molecules limiting the range of structural diversity. Moreover, in certain instances, carbohydrates are not known to cross the blood-brain barrier. On the other hand, in some instances, non-carbohydrate small molecules are less likely to be immunogenic or immunoreactive than are carbohydrates. As used herein, carbohydrates are polyhydroxyaldehydes, polyhydroxyketones and their simple derivatives or larger compounds that can be hydrolyzed into such units. Carbohydrates also include polyhydroxyaldehydes, polyhydroxyketones and their simple derivatives that have been modified such that when they enter cells they are reconverted into polyhydroxyaldehydes, polyhydroxyketones. Carbohydrates also include sugar mimetics such as imino structures and alkaloids that inhibit glycosidases such as Deoxynojirimycin, Castanospermine, Australine, Deoxymannojirimycin, Kifunensen, Swainsonine and Mannostatin (page 709 of Essentials of Glycobiology second edition 2008 CSHL Press, CSH, New York). In certain instances, non-carbohydrate small molecules are organic compounds containing less than 3 linked hydroxyl groups with a molecular weight of less than 2000 Daltons.

Similarly, as discussed herein, in certain embodiments, glycan (e.g., chondroitin sulfate, dermatan sulfate, and/or keratan sulfate) inhibitors described herein are selective inhibitors. In some instances, selective inhibitors are beneficial because they are limited to glycan modifications which limit undesirable or toxic side effects. In certain instances, further restrictions to subsets of glycans, further restrict side effects and makes identification, isolation and tracking the effects of the inhibitors more reliable. In some instances, this makes dose determination more reliable. Selective inhibitors include, e.g.,
a. inhibitors that are selective for a glycan (carbohydrate portion of a molecule) not protein, not nucleic acid, not lipid.
b. Inhibitors that are selective for specific glycans and/or specific glycans including, e.g., inhibitors that are selective for one or more of:
   i. Glycans containing galactose (Gal)
   ii. Glycans containing N-acetylglucosamine (GlcNAc)
   iii. Glycans containing N-acetylgalatosamine (GalNAc)
   iv. Glycans containing mannose (Man)
   v. Glycans containing xylose (Xyl)
   vi. Glycans containing fucose (Fuc)
   vii. Glycans containing sialic acid (Sia)
   viii. Glycans containing GlcNAc and Man
   ix. Glycosaminoglycans, but not N-linked, and/or O-linked, and/or glycopipids
   x. Galactose containing glycosaminoglycans
   xi. GlcNAc containing glycosaminoglycans
   xii. Glycans with 6-O sulfated hexosamines
   xiii. Glycans with 6-O sulfated galactose

In certain embodiments, inhibitors described herein inhibit processes that are late in the biosynthetic pathway of a particular glycan. In some instances, targeting early biosynthetic enzymes eliminates or severely reduces other glycans which could have global effects on protein folding, protein solubility and protein processing. These effects could be extremely toxic or lethal. Thus, in certain instances, targeting late enzymes block modifications that involve more specific receptor binding that is involved in certain cellular adhesion and trafficking interactions. In certain instances, specific interactions involving late pathway enzymes are more readily controlled and under controlled conditions (appropriate dosing) have beneficial effects for a number of diseases. Late in the biosynthetic pathway refers to structures late in the biosynthesis of the target glycan. In the case of chondroitin sulfate and dermatan sulfate, this means after the last biosynthetic step that is shared by other glycans (heparan sulfate). This is the GalNac transferase (GalNAc-TI) that initiates CS and DS synthesis. In the case of keratan sulfate, late in the biosynthetic pathway means biosynthetic enzymes after the synthesis of the underlying core glycan (N-linked or O-linked). The first late biosynthetic enzymes are the beta1,4Gal-TI and the beta1,3GlcNAc-T that synthesizes the backbone of KS I and II.

In certain embodiments, inhibitors described herein are cellularly active (e.g., inhibitors alter the function of a biosynthetic enzyme or a regulator of one in an intact cell in culture or in an intact organism). Generally, modification (Inhibition/promotion) of biosynthesis is accomplished most effectively through a small molecule that can penetrate a cell in order to reach its target.

Targeting these glycans could be through modulators (e.g., inhibitors) acting directly on the relevant biosynthetic enzymes or indirectly on other targets (e.g. protein kinase, phosphatase, transporter, GPCR, ion channel, hormone receptor, protease, etc.) that would alter the structure of the glycans though effects on biosynthetic (anabolic) enzymes or degradative (catabolic) enzymes. In some embodiments glycan biosynthesis modulators (e.g., inhibitors) described herein are direct glycan biosynthesis modulators (e.g., inhibitors) (i.e., such modulators directly affect enzymes involved in the biosynthetic pathway of the glycan). In other embodiments, glycan biosynthesis modulators (e.g., inhibitors) described herein are indirect glycan biosynthesis modulators (e.g., inhibitors) (i.e., such modulators indirectly affect the biosynthesis of the glycan, including upstream modulation of glycan biosynthetic components, enzymes, catalysts, or the like). In certain embodiments, glycan biosynthesis modulators (e.g., inhibitors) described herein are both direct and indirect glycan biosynthesis modulators (e.g., inhibitors)

### Compounds

In some embodiments, glycan (e.g., chondroitin sulfate, dermatan sulfate, and/or keratan sulfate) inhibitors include compounds of Table 7. In specific embodiments, glycan (e.g., chondroitin sulfate, dermatan sulfate, and/or keratan sulfate) inhibitors include, but are not limited to, the following compounds: N-(4-chlorophenyl)-2-[(4-methoxyphenyl)amino]-3,5-dinitrobenzamide (2); 4-bromo-N′[2-(trifluoroacetyl)-1-cyclopenten-1-yl]benzohydrazide (17); 7-[(3-chlorophenyl)(2-pyridinylamino)methyl]-2-methyl-8-quinolinol (59); 7-[(2-fluorophenyl)(2-pyridinylamino)methyl]-2-methyl-8-quinolinol (60); N-{4-[(4-benzyl-1-piperidinyl)carbonyl]-1-phenyl-1H-pyrazol-5-yl}-3-methylbenzamide (98); 7-tert-butyl-2-(trifluoromethyl)-5,6,7,8-tetrahydro[1]benzothieno[2,3-d]pyrimidin-4(3H)-one (99); N-[(8-hydroxy-7-quinolinyl)(4-methylphenyl)methyl]cyclohexanecarboxamide (115); N-[(dibenzylamino)carbonothioyl]-2-fluorobenzamide (181); 3-chloro-N-[(dibenzylamino)carbonothioyl]benzamide (185).

TABLE 7

| # | IUPAC Nomenclature |
|---|---|
| 1 | 2-[(1-bromo-2-naphthyl)oxy]-N′-(3-phenyl-2-propen-1-ylidene)butanohydrazide |
| 2 | N-(4-chlorophenyl)-2-[(4-methoxyphenyl)amino]-3,5-dinitrobenzamide |
| 3 | N-[(diethylamino)carbonothioyl]benzamide |
| 4 | 4-methoxy-N-(4-morpholinylcarbonothioyl)benzamide |
| 5 | 4-methoxy-N-(1-piperidinylcarbonothioyl)benzamide |
| 6 | N′-[2-(allyloxy)benzylidene]-2-[(1-bromo-2-naphthyl)oxy]butanohydrazide |
| 7 | 2-[(1-bromo-2-naphthyl)oxy]-N′-(2-hydroxybenzylidene)butanohydrazide |
| 8 | N′-benzylidene-2-[(1-bromo-2-naphthyl)oxy]butanohydrazide |
| 9 | 2-(3-bromophenoxy)-N′-(4-butoxybenzylidene)butanohydrazide |
| 10 | 2-[(1-bromo-2-naphthyl)oxy]-N′-(2-hydroxy-5-methoxybenzylidene)butanohydrazide |
| 11 | N-(4-thiomorpholinylcarbonothioyl)benzamide |
| 12 | 2-[(1-bromo-2-naphthyl)oxy]-N′-[4-(diethylamino)benzylidene]butanohydrazide |
| 13 | N-[(4-methyl-1-piperazinyl)carbonothioyl]benzamide |
| 14 | N′-(3-allyl-2-hydroxybenzylidene)-2-[(1-bromo-2-naphthyl)oxy]butanohydrazide |
| 15 | 4-chloro-N-{[4-(4-nitrophenyl)-1-piperazinyl]carbonothioyl}benzamide |
| 16 | N-(1-azepanylcarbonothioyl)benzamide |
| 17 | 4-bromo-N′-[2-(trifluoroacetyl)-1-cyclopenten-1-yl]benzohydrazide |
| 18 | N-[1,3-benzodioxol-5-yl(8-hydroxy-7-quinolinyl)methyl]benzamide |
| 19 | N-[(8-hydroxy-7-quinolinyl)(3-nitrophenyl)methyl]acetamide |
| 20 | N-[(8-hydroxy-7-quinolinyl)(phenyl)methyl]butanamide |
| 21 | 4-bromo-N-{[4-(4-nitrophenyl)-1-piperazinyl]carbonothioyl}benzamide |
| 22 | 7-[(4-methylphenyl)(2-pyridinylamino)methyl]-8-quinolinol |
| 23 | 7-[(4-isopropoxyphenyl)(2-pyridinylamino)methyl]-8-quinolinol |
| 24 | 7-[(4-chlorophenyl)(2-pyridinylamino)methyl]-8-quinolinol |
| 25 | 7-{(4-ethoxy-3-methylphenyl)[(4-methyl-2-pyridinyl)amino]methyl}-8-quinolinol |
| 26 | 7-[(2-fluorophenyl)(2-pyridinylamino)methyl]-8-quinolinol |
| 27 | 7-{(4-bromophenyl)[(6-methyl-2-pyridinyl)amino]methyl}-8-quinolinol |
| 28 | 7-{(4-isopropylphenyl)[(4-methyl-2-pyridinyl)amino]methyl}-8-quinolinol |
| 29 | N-[(5-chloro-8-hydroxy-7-quinolinyl)(2-methoxyphenyl)methyl]-3-methoxybenzamide |
| 30 | N-[(5-chloro-8-hydroxy-7-quinolinyl)(3-nitrophenyl)methyl]nicotinamide |
| 31 | N-[(5-chloro-8-hydroxy-7-quinolinyl)(4-methoxyphenyl)methyl]-3-methoxybenzamide |
| 32 | N-[(5-chloro-8-hydroxy-7-quinolinyl)(4-nitrophenyl)methyl]nicotinamide |
| 33 | N-[(5-chloro-8-hydroxy-7-quinolinyl)(4-nitrophenyl)methyl]-2-phenoxyacetamide |
| 34 | N-[(5-chloro-8-hydroxy-7-quinolinyl)(4-methoxyphenyl)methyl]-2-phenoxyacetamide |
| 35 | N-[(5-chloro-8-hydroxy-7-quinolinyl)(3-nitrophenyl)methyl]-4-methoxybenzamide |
| 36 | 2-fluoro-N-({4-[2-nitro-4-(trifluoromethyl)phenyl]-1-piperazinyl}carbonothioyl)benzamide |
| 37 | 2-chloro-5-iodo-N-(4-morpholinylcarbonothioyl)benzamide |
| 38 | 3-methoxy-N-({4-[2-nitro-4-(trifluoromethyl)phenyl]-1-piperazinyl}carbonothioyl)benzamide |
| 39 | 3-iodo-N-{[4-(4-nitrophenyl)-1-piperazinyl]carbonothioyl}benzamide |
| 40 | 4-methoxy-N-(4-morpholinylcarbonothioyl)-3-nitrobenzamide |
| 41 | ethyl 1-{[(4-fluorobenzoyl)amino]carbonothioyl}-4-piperidinecarboxylate |
| 42 | 4-isopropoxy-N-(4-morpholinylcarbonothioyl)benzamide |
| 43 | 2-bromo-N-(4-morpholinylcarbonothioyl)benzamide |
| 44 | 4-methoxy-3-nitro-N-(1-piperidinylcarbonothioyl)benzamide |
| 45 | 2-chloro-N-(4-morpholinylcarbonothioyl)benzamide |
| 46 | 3-methyl-N-{[4-(4-nitrophenyl)-1-piperazinyl]carbonothioyl}benzamide |
| 47 | 4-fluoro-N-{[4-(4-nitrophenyl)-1-piperazinyl]carbonothioyl}benzamide |
| 48 | 2-fluoro-N-{[4-(4-nitrophenyl)-1-piperazinyl]carbonothioyl}benzamide |
| 49 | 7-[[4-(diethylamino)phenyl](2-pyridinylamino)methyl]-2-methyl-8-quinolinol |
| 50 | 7-[(4-methoxyphenyl)(2-pyridinylamino)methyl]-2-methyl-8-quinolinol |
| 51 | 7-{(4-ethoxy-3-methoxyphenyl)[(4-methyl-2-pyridinyl)amino]methyl}-8-quinolinol |
| 52 | 7-[(2-methylphenyl)(2-pyridinylamino)methyl]-8-quinolinol |
| 53 | 2-methyl-7-[phenyl(2-pyridinylamino)methyl]-8-quinolinol |
| 54 | 7-[(3-ethoxyphenyl)(2-pyridinylamino)methyl]-2-methyl-8-quinolinol |
| 55 | 7-[[(4-methyl-2-pyridinyl)amino](2-naphthyl)methyl]-8-quinolinol |
| 56 | 7-[[(4-methyl-2-pyridinyl)amino](4-nitrophenyl)methyl]-8-quinolinol |
| 57 | 7-[(3-bromophenyl)(2-pyridinylamino)methyl]-2-methyl-8-quinolinol |
| 58 | 7-{(4-ethylphenyl)[(4-methyl-2-pyridinyl)amino]methyl}-8-quinolinol |
| 59 | 7-[(3-chlorophenyl)(2-pyridinylamino)methyl]-2-methyl-8-quinolinol |
| 60 | 7-[(2-fluorophenyl)(2-pyridinylamino)methyl]-2-methyl-8-quinolinol |
| 61 | 7-[(4-chlorophenyl)(2-pyridinylamino)methyl]-2-methyl-8-quinolinol |
| 62 | 7-{(2,3-dichlorophenyl)[(4-methyl-2-pyridinyl)amino]methyl}-8-quinolinol |
| 63 | 7-[(4-ethoxyphenyl)(2-pyridinylamino)methyl]-2-methyl-8-quinolinol |
| 64 | 7-{(3-ethoxy-4-methoxyphenyl)[(4-methyl-2-pyridinyl)amino]methyl}-8-quinolinol |
| 65 | 7-{mesityl[(4-methyl-2-pyridinyl)amino]methyl}-8-quinolinol |
| 66 | 2-methyl-7-[(4-methylphenyl)(2-pyridinylamino)methyl]-8-quinolinol |
| 67 | 2-methyl-7-[(4-nitrophenyl)(2-pyridinylamino)methyl]-8-quinolinol |
| 68 | 7-{(2,5-dimethylphenyl)[(4-methyl-2-pyridinyl)amino]methyl}-8-quinolinol |
| 69 | 2-methyl-7-[(2-methylphenyl)(2-pyridinylamino)methyl]-8-quinolinol |
| 70 | 7-[(4-hydroxyphenyl)(2-pyridinylamino)methyl]-2-methyl-8-quinolinol |
| 71 | 7-{[(4-methyl-2-pyridinyl)amino][4-(methylthio)phenyl]methyl}-8-quinolinol |
| 72 | 7-[[(4-methyl-2-pyridinyl)amino](2-nitrophenyl)methyl]-8-quinolinol |
| 73 | 7-[(4-bromophenyl)(2-pyridinylamino)methyl]-2-methyl-8-quinolinol |
| 74 | 4-chloro-N-[(4-methyl-1-piperidinyl)carbonothioyl]benzamide |
| 75 | N-[(2-ethyl-1-piperidinyl)carbonothioyl]-4-fluorobenzamide |
| 76 | N-[(2,6-dimethyl-4-morpholinyl)carbonothioyl]-4-fluorobenzamide |
| 77 | 2,4-dichloro-N-(1-pyrrolidinylcarbonothioyl)benzamide |
| 78 | N-[(2-ethyl-1-piperidinyl)carbonothioyl]-4-nitrobenzamide |
| 79 | N-[(4-methyl-1-piperidinyl)carbonothioyl]-3-nitrobenzamide |
| 80 | N-[(2-ethyl-1-piperidinyl)carbonothioyl]-3-nitrobenzamide |
| 81 | 4-chloro-N-[(2,6-dimethyl-4-morpholinyl)carbonothioyl]benzamide |
| 82 | N-[(2,6-dimethyl-4-morpholinyl)carbonothioyl]benzamide |
| 83 | N-[(2,6-dimethyl-4-morpholinyl)carbonothioyl]-4-nitrobenzamide |
| 84 | N-[(2,6-dimethyl-4-morpholinyl)carbonothioyl]-3-nitrobenzamide |
| 85 | 4-methyl-N-(1-pyrrolidinylcarbonothioyl)benzamide |
| 86 | 4-chloro-N-(1-pyrrolidinylcarbonothioyl)benzamide |
| 87 | N-[(2,6-dimethyl-4-morpholinyl)carbonothioyl]-4-methylbenzamide |
| 88 | 2-bromo-N-(1-pyrrolidinylcarbonothioyl)benzamide |
| 89 | 2-methyl-N-(1-pyrrolidinylcarbonothioyl)benzamide |
| 90 | 2,4-dichloro-N-[(2,6-dimethyl-4-morpholinyl)carbonothioyl]benzamide |
| 91 | 2-methyl-N-[(4-methyl-1-piperidinyl)carbonothioyl]benzamide |
| 92 | N-[(2,6-dimethyl-4-morpholinyl)carbonothioyl]-2-methylbenzamide |
| 93 | 3-nitro-N-(1-pyrrolidinylcarbonothioyl)benzamide |
| 94 | 2-bromo-N-[(2,6-dimethyl-4-morpholinyl)carbonothioyl]benzamide |
| 95 | N-[(4-methyl-1-piperidinyl)carbonothioyl]benzamide |
| 96 | 2-methyl-N-{[4-(4-nitrophenyl)-1-piperazinyl]carbonothioyl}benzamide |
| 97 | N-[(6,7-dimethoxy-3,4-dihydro-2(1H)-isoquinolinyl)carbonothioyl]benzamide |
| 98 | N-{4-[(4-benzyl-1-piperidinyl)carbonyl]-1-phenyl-1H-pyrazol-5-yl}-3-methylbenzamide |
| 99 | 7-tert-butyl-2-(trifluoromethyl)-5,6,7,8-tetrahydro[1]benzothieno[2,3-d]pyrimidin-4(3H)-one |
| 100 | N-[(8-hydroxy-7-quinolinyl)(4-isopropylphenyl)methyl]cyclohexanecarboxamide |
| 101 | N-[(5-chloro-8-hydroxy-7-quinolinyl)(4-chlorophenyl)methyl]butanamide |
| 102 | N-[(2,4-dichlorophenyl)(8-hydroxy-5-nitro-7-quinolinyl)methyl]butanamide |
| 103 | N-[(5-chloro-8-hydroxy-7-quinolinyl)(2-chlorophenyl)methyl]propanamide |
| 104 | N-[(5-chloro-8-hydroxy-7-quinolinyl)(3,4-dimethoxyphenyl)methyl]acetamide |
| 105 | N-[(3-ethoxy-4-hydroxyphenyl)(8-hydroxy-5-nitro-7-quinolinyl)methyl]butanamide |
| 106 | 2-chloro-N-[(2,6-dimethyl-4-morpholinyl)carbonothioyl]benzamide |
| 107 | N-[(8-hydroxy-5-nitro-7-quinolinyl)(2-methoxyphenyl)methyl]pentanamide |
| 108 | N-[(5-chloro-8-hydroxy-7-quinolinyl)(4-methylphenyl)methyl]acetamide |
| 109 | N-[(8-hydroxy-5-nitro-7-quinolinyl)(4-methoxyphenyl)methyl]pentanamide |
| 110 | N-[(4-chlorophenyl)(8-hydroxy-5-nitro-7-quinolinyl)methyl]acetamide |
| 111 | N-[(2,4-dichlorophenyl)(8-hydroxy-7-quinolinyl)methyl]acetamide |
| 112 | N-[(8-hydroxy-5-nitro-7-quinolinyl)(phenyl)methyl]pentanamide |
| 113 | N-[(4-chlorophenyl)(8-hydroxy-7-quinolinyl)methyl]cyclohexanecarboxamide |
| 114 | N-[(8-hydroxy-7-quinolinyl)(4-methylphenyl)methyl]cyclohexanecarboxamide |
| 115 | N-(4,5-diphenyl-1,3-oxazol-2-yl)-4-fluorobenzamide |
| 116 | N-[1,3-benzodioxol-5-yl(8-hydroxy-5-nitro-7-quinolinyl)methyl]pentanamide |
| 117 | N-[(2,4-dichlorophenyl)(8-hydroxy-7-quinolinyl)methyl]-2-methylpropanamide |
| 118 | N-[(dibenzylamino)carbonothioyl]-2-methylbenzamide |
| 119 | N-[[4-(diethylamino)phenyl](8-hydroxy-7-quinolinyl)methyl]pentanamide |
| 120 | 2-chloro-N-[(4-methyl-1-piperidinyl)carbonothioyl]benzamide |
| 121 | N-[(4-chlorophenyl)(8-hydroxy-5-nitro-7-quinolinyl)methyl]pentanamide |
| 122 | N-[(dibenzylamino)carbonothioyl]-4-fluorobenzamide |
| 123 | 2-chloro-N-(1-pyrrolidinylcarbonothioyl)benzamide |
| 124 | N-[(dibenzylamino)carbonothioyl]-3-nitrobenzamide |
| 125 | N-[(8-hydroxy-7-quinolinyl)(4-isopropylphenyl)methyl]acetamide |
| 126 | N-[(5-chloro-8-hydroxy-7-quinolinyl)(4-chlorophenyl)methyl]propanamide |
| 127 | N-[(5-chloro-8-hydroxy-7-quinolinyl)(2,4-dichlorophenyl)methyl]acetamide |
| 128 | N-[(4-chlorophenyl)(8-hydroxy-5-nitro-7-quinolinyl)methyl]butanamide |
| 129 | N-[(5-chloro-8-hydroxy-7-quinolinyl)(2-methoxyphenyl)methyl]propanamide |
| 130 | N-[(8-hydroxy-5-nitro-7-quinolinyl)(phenyl)methyl]acetamide |
| 131 | N-[(dibenzylamino)carbonothioyl]-4-nitrobenzamide |
| 132 | N-[(8-hydroxy-5-nitro-7-quinolinyl)(2-methoxyphenyl)methyl]propanamide |
| 133 | N-[(3,4-dimethoxyphenyl)(8-hydroxy-5-nitro-7-quinolinyl)methyl]pentanamide |
| 134 | N-[(8-hydroxy-5-nitro-7-quinolinyl)(4-isopropylphenyl)methyl]butanamide |
| 135 | N-[(5-chloro-8-hydroxy-7-quinolinyl)(4-isopropylphenyl)methyl]acetamide |
| 136 | N-[(5-chloro-8-hydroxy-7-quinolinyl)(3,4-dimethoxyphenyl)methyl]propanamide |
| 137 | N-[(8-hydroxy-7-quinolinyl)(2-methoxyphenyl)methyl]cyclohexanecarboxamide |
| 138 | N-[(dibenzylamino)carbonothioyl]-4-methylbenzamide |
| 139 | N-[(2-chlorophenyl)(8-hydroxy-5-nitro-7-quinolinyl)methyl]pentanamide |
| 140 | N-[(3-ethoxy-4-hydroxyphenyl)(8-hydroxy-5-nitro-7-quinolinyl)methyl]acetamide |
| 141 | N-[(2,4-dichlorophenyl)(8-hydroxy-7-quinolinyl)methyl]pentanamide |
| 142 | N-[(8-hydroxy-5-nitro-7-quinolinyl)(4-methylphenyl)methyl]propanamide |
| 143 | N-[(5-chloro-8-hydroxy-7-quinolinyl)(4-methoxyphenyl)methyl]acetamide |
| 144 | 2-chloro-N-[(2-ethyl-1-piperidinyl)carbonothioyl]benzamide |
| 145 | N-[[4-(diethylamino)phenyl](8-hydroxy-7-quinolinyl)methyl]-2-methylpropanamide |
| 146 | N-{(5-chloro-8-hydroxy-7-quinolinyl)[4-(dimethylamino)phenyl]methyl}acetamide |
| 147 | 7-[(4-ethoxy-3-methoxyphenyl)(2-pyridinylamino)methyl]-2-methyl-8-quinolinol |
| 148 | 7-[(2-chlorophenyl)(2-pyridinylamino)methyl]-2-methyl-8-quinolinol |
| 149 | 7-{(3-hydroxy-4-methoxyphenyl)[(4-methyl-2-pyridinyl)amino]methyl}-8-quinolinol |
| 150 | 7-[(4-methoxy-3-methylphenyl)(2-pyridinylamino)methyl]-2-methyl-8-quinolinol |
| 151 | 7-[(2,5-dimethylphenyl)(2-pyridinylamino)methyl]-2-methyl-8-quinolinol |
| 152 | 7-[(3-phenoxyphenyl)(2-pyridinylamino)methyl]-8-quinolinol |
| 153 | 7-[(4-isopropylphenyl)(2-pyridinylamino)methyl]-2-methyl-8-quinolinol |
| 154 | 7-[(2-chloro-6-fluorophenyl)(2-pyridinylamino)methyl]-2-methyl-8-quinolinol |
| 155 | N-[(5-chloro-8-hydroxy-7-quinolinyl)(4-isopropylphenyl)methyl]-2-methylpropanamide |
| 156 | N-[(5-chloro-8-hydroxy-7-quinolinyl)(4-chlorophenyl)methyl]butanamide |
| 157 | N-[(5-chloro-8-hydroxy-7-quinolinyl)(4-methoxyphenyl)methyl]pentanamide |
| 158 | N-[(4-phenyl-1-piperazinyl)carbonothioyl]benzamide |
| 159 | N-[(8-hydroxy-5-nitro-7-quinolinyl)(phenyl)methyl]-2-methylpropanamide |
| 160 | N-[(3,4-dimethoxyphenyl)(8-hydroxy-5-nitro-7-quinolinyl)methyl]-2-methylpropanamide |
| 161 | N-[1,3-benzodioxol-5-yl(8-hydroxy-7-quinolinyl)methyl]-3-phenylpropanamide |
| 162 | N-[(2,4-dichlorophenyl)(8-hydroxy-7-quinolinyl)methyl]-3-methylbutanamide |
| 163 | N-[1,3-benzodioxol-5-yl(8-hydroxy-5-nitro-7-quinolinyl)methyl]-2-methylpropanamide |
| 164 | N-[(8-hydroxy-7-quinolinyl)(4-methylphenyl)methyl]-3-phenylpropanamide |
| 165 | N-{(5-chloro-8-hydroxy-7-quinolinyl)[4-(dimethylamino)phenyl]methyl}butanamide |
| 166 | N-[1,3-benzodioxol-5-yl(8-hydroxy-5-nitro-7-quinolinyl)methyl]cyclohexanecarboxamide |
| 167 | N-[(3,4-dimethoxyphenyl)(8-hydroxy-5-nitro-7-quinolinyl)methyl]cyclohexanecarboxamide |
| 168 | N-[(2-chlorophenyl)(8-hydroxy-5-nitro-7-quinolinyl)methyl]propanamide |
| 169 | N-[(5-chloro-8-hydroxy-7-quinolinyl)(2-methoxyphenyl)methyl]butanamide |
| 170 | N-[(8-hydroxy-5-nitro-7-quinolinyl)(4-isopropylphenyl)methyl]-2-methylpropanamide |
| 171 | N-[(2,6-dimethyl-4-morpholinyl)carbonothioyl]-4-methoxybenzamide |
| 172 | N-{(5-chloro-8-hydroxy-7-quinolinyl)[4-(diethylamino)phenyl]methyl}butanamide |
| 173 | N-[(dibenzylamino)carbonothioyl]-4-methoxybenzamide |
| 174 | N-[(8-hydroxy-5-nitro-7-quinolinyl)(4-methoxyphenyl)methyl]-2-methylpropanamide |
| 175 | N-[(4-chlorophenyl)(8-hydroxy-5-nitro-7-quinolinyl)methyl]-2-methylpropanamide |
| 176 | N-[(4-chlorophenyl)(8-hydroxy-5-nitro-7-quinolinyl)methyl]cyclohexanecarboxamide |
| 177 | N-[(3,4-dimethoxyphenyl)(8-hydroxy-7-quinolinyl)methyl]-3-phenylpropanamide |
| 178 | N-[(4-chlorophenyl)(8-hydroxy-7-quinolinyl)methyl]-3-phenylpropanamide |
| 179 | N-{[4-(diphenylmethyl)-1-piperazinyl]carbonothioyl}benzamide |
| 180 | N-[(dibenzylamino)carbonothioyl]-2-fluorobenzamide |
| 181 | N-[(8-hydroxy-5-nitro-7-quinolinyl)(2-thienyl)methyl]-2-methylpropanamide |
| 182 | N-[(5-chloro-8-hydroxy-7-quinolinyl)(phenyl)methyl]pentanamide |
| 183 | N-[(3-ethoxy-4-hydroxyphenyl)(8-hydroxy-5-nitro-7-quinolinyl)methyl]pentanamide |
| 184 | 3-chloro-N-[(dibenzylamino)carbonothioyl]benzamide |
| 185 | 3-chloro-N-[(dibenzylamino)carbonothioyl]benzamide |
| 186 | N-[(8-hydroxy-5-nitro-7-quinolinyl)(2-methoxyphenyl)methyl]cyclohexanecarboxamide |
| 187 | N-[(3,4-dimethoxyphenyl)(8-hydroxy-7-quinolinyl)methyl]-3-methylbutanamide |
| 188 | 4-methoxy-N-[(4-methyl-1-piperidinyl)carbonothioyl]benzamide |
| 189 | 7-[(3,4-dichlorophenyl)(2-pyridinylamino)methyl]-2-methyl-8-quinolinol |
| 190 | N-[(1-methyl-1,3,4,9-tetrahydro-2H-beta-carbolin-2-yl)carbonothioyl]benzamide |
| 191 | 7-{(3,4-diethoxyphenyl)[(4-methyl-2-pyridinyl)amino]methyl}-8-quinolinol |
| 192 | 7-{[3-(allyloxy)phenyl][(4-methyl-2-pyridinyl)amino]methyl}-8-quinolinol |
| 193 | 7-[[(4-methyl-2-pyridinyl)amino](4-propoxyphenyl)methyl]-8-quinolinol |
| 194 | 7-{(4-isopropoxyphenyl)[(4-methyl-2-pyridinyl)amino]methyl}-8-quinolinol |
| 195 | 2-methyl-7-{(2-methylphenyl)[(6-methyl-2-pyridinyl)amino]methyl}-8-quinolinol |
| 196 | 2-iodo-N-[(4-methyl-1-piperazinyl)carbonothioyl]benzamide |
| 197 | 2-chloro-N-{[4-(2-methoxyphenyl)-1-piperazinyl]carbonothioyl}benzamide |

### General Definitions

The term “subject”, “patient” or “individual” are used interchangeably herein and refer to mammals and non-mammals, e.g., suffering from a disorder described herein. Examples of mammals include, but are not limited to, any member of the Mammalian class humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the patient.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to a patient at risk of developing a particular disease, to a patient reporting one or more of the physiological symptoms of a disease, or to a patient at risk of reoccurrence of the disease.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent. Furthermore, combination treatments are optionally administered separately or concomitantly.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to a patient simultaneously in the form of a single entity or dosage. The term “non-fixed combination” means that at least one of the agents described herein, and at least one co-agent, are administered to a patient as separate entities either simultaneously, concurrently or sequentially with variable intervening time limits, wherein such administration provides effective levels of the two or more agents in the body of the patient. In some instances, the co-agent is administered once or for a period of time, after which the agent is administered once or over a period of time. In other instances, the co-agent is administered for a period of time, after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration”, “administered in combination with” and their grammatical equivalents are meant to encompass administration of the selected therapeutic agents to a single patient, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is the alteration of or the disruption of the structure of endogenous glycan such that the binding ability, signaling ability or combination thereof of the glycan is inhibited or reduced. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the agents and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “carrier” as used herein, refers to relatively nontoxic chemical agents that, in certain instances, facilitate the incorporation of an agent into cells or tissues.

“Pharmaceutically acceptable prodrug” as used herein, refers to any pharmaceutically acceptable salt, ester, salt of an ester or other derivative of an agent, which, upon administration to a recipient, is capable of providing, either directly or indirectly, a chondroitin sulflate modulator agent described herein or a pharmaceutically active metabolite or residue thereof. Particularly favored prodrugs are those that increase the bioavailability of the glycan modulator agents described herein when such agents are administered to a patient (e.g., by allowing an orally administered agent to be more readily absorbed into blood) or which enhance delivery of the parent agent to a biological compartment (e.g., the brain or lymphatic system). In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-toluenenesulfonate, mesylate and the like. Furthermore, pharmaceutically acceptable salts include, by way of non-limiting example, alkaline earth metal salts (e.g., calcium or magnesium), alkali metal salts (e.g., sodium or potassium), ammonium salts and the like.

### Methods

Provided in certain embodiments herein is a process for modifying the structure of glycan described herein (e.g., chondroitin sulfate, dermatan sulfate, and/or keratan sulfate) on a core protein (i.e., a proteoglycan, such as a chondroitin sulfate proteoglycan), comprising contacting a cell that translationally produces at least one core protein having at least one attached glycan moiety with an effective amount of any glycan (e.g., chondroitin sulfate, dermatan sulfate, and/or keratan sulfate) inhibitor described herein. In some embodiments, the glycan (e.g., chondroitin sulfate, dermatan sulfate, and/or keratan sulfate) inhibitor is a selective glycan (e.g., chondroitin sulfate, dermatan sulfate, and/or keratan sulfate) inhibitor (as compared to the inhibition of the function of other glycans or GAGs), e.g., as described herein. In some embodiments, the selective glycan (e.g., chondroitin sulfate, dermatan sulfate, and/or keratan sulfate) inhibitor is a modulator of (e.g., promotes one or more of, or inhibits one or more of) glycan (e.g., chondroitin sulfate, dermatan sulfate, and/or keratan sulfate) glycosylation (e.g., modulates a chondroitin sulfate glycosyltransferase), glycan (e.g., chondroitin sulfate, dermatan sulfate, and/or keratan sulfate) sulfation (e.g., modulates a chondroitin sulfate sulfotransferase), glycan (e.g., chondroitin sulfate, dermatan sulfate, and/or keratan sulfate) phosphorylation (e.g., modulates a chondroitin sulfate kinase) or a combination thereof.

In some embodiments, the glycan (e.g., chondroitin sulfate, dermatan sulfate, and/or keratan sulfate) inhibitor modulates (e.g., promote or inhibit) glycosyltransferase. In some embodiments, the inhibitor of a glycan (e.g., chondroitin sulfate, dermatan sulfate, and/or keratan sulfate) glycosyltransferase inhibits the synthesis of the linkage region, the initiation of glycan (e.g., chondroitin sulfate, dermatan sulfate, and/or keratan sulfate) synthesis, the synthesis of glycan (e.g., chondroitin sulfate, dermatan sulfate, and/or keratan sulfate), or a combination thereof. In some embodiments, glycan (e.g., chondroitin sulfate, dermatan sulfate, and/or keratan sulfate) inhibitors modulate (e.g., promote or inhibit) one or more of a xylosyltransfarase (e.g., chondroitin sulfate xylosyltransfarase), a galactosyltransferase (e.g., chondroitin sulfate galactosyltransferase), a glucuronosyltransferase (e.g., chondroitin sulfate glucuronosyltransferase), an N-acetylgalactosaminyl transferase (e.g., chondroitin sulfate N-acetylgalactosaminyl transferase), or combinations thereof. In more specific embodiments, glycan (e.g., chondroitin sulfate, dermatan sulfate, and/or keratan sulfate) inhibitors selectively modulate (e.g., promote or inhibit) one or more of xylosyltransfarase I, xylosyltransfarase II, galactosyltransferase I, galactosyltransferase II, glucuronosyltransferase I, glucuronosyltransferase II, N-acetylgalactosaminyl transferase I, N-acetylgalactosaminyl transferase II, or a combination thereof.

In certain embodiments, chondroitin sulfate inhibitors that modulate sulfation modulate one or more sulfotransferase. In specific embodiments, the sulfotransferase is, by way of non-limiting example, a modulator (e.g., inhibitor or promoter) of one or more of a chondroitin sulfate O-sulfotransferase. In more specific embodiments, the chondroitin sulfate inhibitor modulates (e.g., inhibits or promotes) a chondroitin sulfate O-sulfotransferase such as, by way of non-limiting example, one or more of a 6-O sulfotransferase (of a galactosaminyl group), a 4-O sulfotransferase (of a galactosaminyl group), a 2-O sulfotransferase (of a uronic acid moiety, e.g., glucuronic acid), or a combination thereof.

In certain embodiments, the effective amount of the glycan (e.g., chondroitin sulfate, dermatan sulfate, and/or keratan sulfate) inhibitor alters or disrupts the nature (e.g., alters or disrupts the acetylation, sulfation, O-sulfation, the 2-O sulfation, the 4-O sulfation, the 6-O sulfation, the concentration of glycan (e.g., chondroitin sulfate, dermatan sulfate, and/or keratan sulfate), chain length of chondroitin sulfate, or a combination thereof) of glycan (e.g., chondroitin sulfate, dermatan sulfate, and/or keratan sulfate) compared to endogenous glycan (e.g., chondroitin sulfate, dermatan sulfate, and/or keratan sulfate) in an amount sufficient to alter or disrupt glycan (e.g., chondroitin sulfate, dermatan sulfate, and/or keratan sulfate) binding, glycan (e.g., chondroitin sulfate, dermatan sulfate, and/or keratan sulfate) signaling, or a combination thereof. In specific embodiments, the glycan (e.g., chondroitin sulfate, dermatan sulfate, and/or keratan sulfate) inhibitor described herein alters or disrupts the nature of the glycan (e.g., chondroitin sulfate, dermatan sulfate, and/or keratan sulfate) such that it inhibits glycan (e.g., chondroitin sulfate, dermatan sulfate, and/or keratan sulfate) signaling. In other specific embodiments, the glycan (e.g., chondroitin sulfate, dermatan sulfate, and/or keratan sulfate) inhibitor described herein alters or disrupts the nature of the glycan (e.g., chondroitin sulfate, dermatan sulfate, and/or keratan sulfate) such that it inhibits glycan (e.g., chondroitin sulfate, dermatan sulfate, and/or keratan sulfate) binding. In more specific embodiments, the glycan (e.g., chondroitin sulfate, dermatan sulfate, and/or keratan sulfate) inhibitor described herein alters or disrupts the nature of the glycan (e.g., chondroitin sulfate, dermatan sulfate, and/or keratan sulfate) such that it inhibits glycan (e.g., chondroitin sulfate, dermatan sulfate, and/or keratan sulfate) binding and glycan (e.g., chondroitin sulfate, dermatan sulfate, and/or keratan sulfate) signaling. In some embodiments, the glycan (e.g., chondroitin sulfate, dermatan sulfate, and/or keratan sulfate) inhibitor alters or disrupts the nature of the glycan (e.g., chondroitin sulfate, dermatan sulfate, and/or keratan sulfate) such that it inhibits the binding, singaling, or a combination thereof of any lectin (including polypeptides) subject to glycan (e.g., chondroitin sulfate, dermatan sulfate, and/or keratan sulfate) binding, signaling or a combination thereof, in the absence of a glycan (e.g., chondroitin sulfate, dermatan sulfate, and/or keratan sulfate) inhibitor. In some embodiments, the lectin is, by way of non-limiting example, a growth factor. In specific embodiments, the growth factor is (e.g., for chondroitin sulfate and/or dermatan sulfate binding or signaling), by way of non-limiting example, pleiotrophin, midkine, vascular endothelial growth factor (VEGF), fibroblast growth factor (FGF), hepatocyte growth factor, heparin co-factor II or heparin-binding epidermal growth factor (HB-EGF). In other specific embodiments (e.g., for keratan sulfate binding or signaling), the lectin is 5-D-4, or galectins. In certain embodiments, the glycan (e.g., chondroitin sulfate, dermatan sulfate, and/or keratan sulfate) inhibitor described herein selectively affects (e.g., inhibits) the binding of one or more of the afore mentioned lectins, but does not affect (e.g., inhibit) the binding of one or more of WGA, MAL, PHA, ConA, FGF2, CTB (cholera toxin B-subunit).

In certain embodiments, the cell is present in an individual (e.g., a human) diagnosed with a disorder mediated by glycan (e.g., chondroitin sulfate, dermatan sulfate, and/or keratan sulfate). In certain instances, the disorder mediated by glycan (e.g., chondroitin sulfate, dermatan sulfate, and/or keratan sulfate) is a cancer, a tumor, a lysosomal storage disease (e.g., MPS, etc.), undesired angiogenesis (e.g., cancer, diabetic blindness, age-related macular degeneration, rheumatoid arthritis, or psoriasis), insufficient angiogenesis (e.g., coronary artery disease, stroke, or delayed wound healing), mucopolysaccharidosis, amyloidosis, a spinal cord injury, hypertriglyceridemia, inflammation, diseases associated with inflammation, a wound, or the like. In some embodiments, the cell is present in a human diagnosed with cancer. In certain embodiments, the cell is present in an individual (e.g., a human) diagnosed with abnormal angiogenesis and/or undesired angiogenesis. In some embodiments, the cell is present in an individual (e.g., a human) diagnosed with a lysosomal storage disease (e.g., mucopolysaccharidosis (MPS)). In specific embodiments, the individual is diagnosed with MPS IV, MPS VI, MPS VII, MPS I or MPS II. In some embodiments, the cell is present in an individual (e.g., a human) diagnosed with amyloidosis, a spinal cord injury, hypertriglyceridemia, inflammation, neurodegenerative diseases, or the like. In specific embodiments, the cell is present in an individual suffering from a spinal cord injury. In some embodiments, the cell is present in an individual suffering from a neurodegenerative disease.

In some embodiments, the cell is present in an individual (e.g., a human) diagnosed with Alzheimer's disease, Parkinson's disease, Huntington's disease, spongiform encephalopathies (Creutzfeld-Jakob, Kuru, Mad Cow), diabetic amyloidosis, type-2 diabetes, Rheumatoid arthritis, juvenile chronic arthritis, Ankylosing spondylitis, psoriasis, psoriatic arthritis, adult still disease, Becet syndrome, famalial Mediterranean fever, Crohn's disease, leprosy, osteomyelitis, tuberculosis, chronic bronciectasis, Castleman disease, Hodgkin's disease, renal cell carcinoma, or carcinoma of the gut, lung or urogenital tract. In some embodiments, a disease treated or a cell is present in is neural regeneration, e.g. stroke. Other diseases include, e.g., dermal reconstruction and joint reconstruction.

In some embodiments, the cell is present in an individual (e.g., human) diagnosed with prostate cancer, pancreatic cancer, myoloma, gastric cancer, ovarian cancer, hepatocellular cancer, breast cancer, colon carcinoma, or melanoma. In certain embodimens, the cell is a prostate cancer cell, pancreatic cancer cell, myoloma cell, ovarian cancer cell, hepatocellular cancer cell, breast cancer cell, colon carcinoma cell, renal cell carcinoma, carcinoma of the gut, lung or urogenital tract, or melanoma cell.

In some embodiments, the cell is present in an individual (e.g., human) diagnosed with an infectious or viral disease including, by way of non-limiting example, herpes, diphtheria, papilloma virus, hepatitis, HIV, coronavirus, or adenovirus.

In certain embodiments, chondroitin sulfate inhibitors described herein are small molecule organic compounds. In certain instances, chondroitin sulfate inhibitors utilized herein are not polypeptides or carbohydrates. In some embodiments, a small molecule organic compound has a molecular weight of less than 2,000 g/mol, less than 1,500 g/mol, less than 1,000 g/mol, less than 700 g/mol, or less than 500 g/mol.

In certain embodiments, provided herein is a method of treating a disorder mediated by one or more glycosaminoglycan comprising galactose or N-acetylgalactosamine (e.g., chondroitin sulfate, dermatan sulfate, and/or keratan sulfate) by administering to an individual (e.g., a human) in need thereof a therapeutically effective amount of any glycan (e.g., chondroitin sulfate, dermatan sulfate, and/or keratan sulfate) inhibitor described herein. In specific embodiments, the glycan (e.g., chondroitin sulfate, dermatan sulfate, and/or keratan sulfate) inhibitor is a modulator (e.g., inhibitor or promoter) of a glycan (e.g., chondroitin sulfate, dermatan sulfate, and/or keratan sulfate) glycosyltransferase, or glycan (e.g., chondroitin sulfate, dermatan sulfate, and/or keratan sulfate) sulfotransferase. In certain instances, the disorder mediated by a glycosaminoglycan comprising galactose or N-acetylgalactosamine (e.g., chondroitin sulfate, dermatan sulfate, and/or keratan sulfate) is a cancer, a tumor, undesired angiogenesis (e.g., cancer, diabetic blindness, age-related macular degeneration, rheumatoid arthritis, or psoriasis), insufficient angiogenesis (e.g., coronary artery disease, stroke, or delayed wound healing), mucopolysaccharidosis, amyloidosis, a spinal cord injury, hypertriglyceridemia, inflammation, disease associated with inflammation, lysosomal storage disease (e.g., MPS, or the like), a wound, or the like. In some embodiments, provided herein is a method of treating cancer by administering to an individual (e.g., a human) in need thereof a therapeutically effective amount of any chondroitin sulfate inhibitor described herein. In some embodiments, provided herein is a method of treating a tumor by administering to an individual (e.g., a human) in need thereof a therapeutically effective amount of any chondroitin sulfate inhibitor described herein. In some embodiments, provided herein is a method of treating undesired angiogenesis by administering to an individual (e.g., a human) in need thereof a therapeutically effective amount of any chondroitin sulfate inhibitor described herein. In some embodiments, provided herein is a method of treating a lysosomal storage disease (e.g., MPS) by administering to an individual (e.g., a human) in need thereof a therapeutically effective amount of any chondroitin sulfate inhibitor described herein. In some embodiments, provided herein is a method of treating a amyloidosis, a spinal cord injury, hypertriglyceridemia, and/or inflammation by administering to an individual (e.g., a human) in need thereof a therapeutically effective amount of any chondroitin sulfate inhibitor described herein.

In certain embodiments, provided herein is a method of treating a spinal cord injury by administering to an individual (e.g., human) a therapeutically effective amount of any chondroitin sulfate modulator (e.g., inhibitor) described herein. In some embodiments, provided herein is a method of treating a neurodegenerative disease by administering to an individual (e.g., human) a therapeutically effective amount of any chondroitin sulfate modulator (e.g., inhibitor) described herein.

In some embodiments, provided herein is a method of treating cancer by administering to an individual (e.g., human) a therapeutically effective amount of any chondroitin sulfate inhibitor described herein. In some embodiments, the cancer is, by way of non-limiting example, prostate cancer, pancreatic cancer, myoloma, gastric cancer, ovarian cancer, hepatocellular cancer, breast cancer, colon carcinoma, renal cell carcinoma, carcinoma of the gut, lung or urogenital tract, or melanoma.

In some embodiments, provided herein is a method of treating an infectious or viral disease by administering to an individual (e.g., human) a therapeutically effective amount of any chondroitin sulfate inhibitor described herein. In some embodiments, the infectious or viral disease includes, by way of non-limiting example, herpes, diphtheria, papilloma virus, hepatitis, HIV, coronavirus, or adenovirus.

In some embodiments, the treatment of amyloidosis includes the treatment of Alzheimer's disease, Parkinson's disease, type-2 diabetes, Huntington's disease, spongiform encephalopathies (Creutzfeld-Jakob, Kuru, Mad Cow), diabetic amyloidosis, Rheumatoid arthritis, juvenile chronic arthritis, Ankylosing spondylitis, psoriasis, psoriatic arthritis, adult still disease, Becet syndrom, famalial Mediterranean fever, Crohn's disease, leprosy, osteomyelitis, tuberculosis, chronic bronciectasis, Castleman disease, Hodgkin's diease, renal cell carcinoma, carcinoma of the gut, lung or urogenital tract.

Provided in certain embodiments herein is a process of inhibiting glycan (e.g., chondroitin sulfate, dermatan sulfate, and/or keratan sulfate) function in a cell comprising contacting the cell with a selective modulator (e.g., with respect to other glycans, specifically GAGs) of galactosamine and glucuronic acid containing glycan (e.g., chondroitin sulfate, dermatan sulfate, and/or keratan sulfate) biosynthesis. In various embodiments, glycan (e.g., chondroitin sulfate, dermatan sulfate, and/or keratan sulfate) biosynthesis, as used herein, includes, by way of non-limiting example, (1) inhibition of (a) glycan (e.g., chondroitin sulfate, dermatan sulfate, and/or keratan sulfate) glycosylation; (b) glycan (e.g., chondroitin sulfate, dermatan sulfate, and/or keratan sulfate) sulfation; and/or (c) glycan (e.g., chondroitin sulfate, dermatan sulfate, and/or keratan sulfate) phosphorylation; and/or (2) promotion of (a) glycan (e.g., chondroitin sulfate, dermatan sulfate, and/or keratan sulfate) bond cleavage; (b) bond cleavage of the linker region connecting glycan (e.g., chondroitin sulfate, dermatan sulfate, and/or keratan sulfate) to a core protein; (c) bond cleavage between glycan (e.g., chondroitin sulfate, dermatan sulfate, and/or keratan sulfate) and the linker region; (d) sulfation of glycan (e.g., chondroitin sulfate, dermatan sulfate, and/or keratan sulfate), and/or (e) glycan (e.g., chondroitin sulfate, dermatan sulfate, and/or keratan sulfate) phosphorylation. In specific embodiments, the modulator of glycan (e.g., chondroitin sulfate, dermatan sulfate, and/or keratan sulfate) biosynthesis inhibits sulfation of a glycan (e.g., chondroitin sulfate, dermatan sulfate, and/or keratan sulfate). In specific embodiments, the modulator of glycan (e.g., chondroitin sulfate, dermatan sulfate, and/or keratan sulfate) biosynthesis promotes sulfation of a glycan (e.g., chondroitin sulfate, dermatan sulfate, and/or keratan sulfate).

In some embodiments, the modulator of glycan (e.g., chondroitin sulfate, dermatan sulfate, and/or keratan sulfate) biosynthesis modulates (e.g., promotes or inhibits) glycosyltransferase. In some embodiments, the modulator of glycan (e.g., chondroitin sulfate, dermatan sulfate, and/or keratan sulfate) glycosyltransferase inhibits the synthesis of the linkage region suitable for connecting glycan (e.g., chondroitin sulfate, dermatan sulfate, and/or keratan sulfate) to a core protein, the initiation of glycan (e.g., chondroitin sulfate, dermatan sulfate, and/or keratan sulfate) synthesis, the synthesis of glycan (e.g., chondroitin sulfate, dermatan sulfate, and/or keratan sulfate), or a combination thereof. In some embodiments, modulators of glycan (e.g., chondroitin sulfate, dermatan sulfate, and/or keratan sulfate) biosynthesis modulate (e.g., promote or inhibit) one or more of a chondroitin sulfate xylosyltransfarase, a chondroitin sulfate galactosyltransferase, a chondroitin sulfate glucuronosyltransferase, a chondroitin sulfate N-acetylgalactosaminyl transferase, or combinations thereof. In more specific embodiments, chondroitin sulfate inhibitors selectively modulate (e.g., promote or inhibit) one or more of xylosyltransfarase I, xylosyltransfarase II, galactosyltransferase I, galactosyltransferase II, glucuronosyltransferase I, glucuronosyltransferase II, N-acetylgalactosaminyl transferase I, N-acetylgalactosaminyl transferase II, or a combination thereof.

In certain embodiments, modulators of glycan (e.g., chondroitin sulfate, dermatan sulfate, and/or keratan sulfate) biosynthesis that modulate sulfation modulate one or more sulfotransferase. In specific embodiments, the sulfotransferase is, by way of non-limiting example, a modulator (e.g., inhibitor or promoter) of one or more of a chondroitin sulfate O-sulfotransferase. In more specific embodiments, the chondroitin sulfate inhibitor modulates (e.g., inhibits or promotes) a chondroitin sulfate O-sulfotransferase such as, by way of non-limiting example, one or more of a 6-O sulfotransferase (of a galactosaminyl group), a 4-O sulfotransferase (of a galactosaminyl group), a 2-O sulfotransferase (of a uronic acid moiety, e.g., glucuronic acid), a 6-O sulfotransferase (of a galactose in the linkage tetrasacchride), a 4-O sulfotransferase (of a galactose in the linkage tetrasacchride) or a combination thereof. In some embodiments, modulators of chondroitin sulfate biosynthesis modulate 2-O phosphorylation of the xylose in the chondroitin sulfate linkage region.

In certain embodiments, the effective amount of the modulator of chondroitin sulfate biosynthesis alters or disrupts the nature (e.g., alters or disrupts the sulfation, O-sulfation, the 2-O sulfation, the 4-O sulfation, the 6-O sulfation, concentration of chondroitin sulfate, chain length of chondroitin sulfate, or a combination thereof) of glycan (e.g., chondroitin sulfate, dermatan sulfate, and/or keratan sulfate) compared to endogenous glycan (e.g., chondroitin sulfate, dermatan sulfate, and/or keratan sulfate) in an amount sufficient to alter or disrupt glycan (e.g., chondroitin sulfate, dermatan sulfate, and/or keratan sulfate) binding, glycan (e.g., chondroitin sulfate, dermatan sulfate, and/or keratan sulfate) signaling, or a combination thereof. In specific embodiments, the modulator of glycan (e.g., chondroitin sulfate, dermatan sulfate, and/or keratan sulfate) biosynthesis alters or disrupts the nature of the glycan (e.g., chondroitin sulfate, dermatan sulfate, and/or keratan sulfate) such that it inhibits glycan (e.g., chondroitin sulfate, dermatan sulfate, and/or keratan sulfate) signaling. In other specific embodiments, the modulator of glycan (e.g., chondroitin sulfate, dermatan sulfate, and/or keratan sulfate) biosynthesis alters or disrupts the nature of the glycan (e.g., chondroitin sulfate, dermatan sulfate, and/or keratan sulfate) such that it inhibits glycan (e.g., chondroitin sulfate, dermatan sulfate, and/or keratan sulfate) binding. In more specific embodiments, modulator of glycan (e.g., chondroitin sulfate, dermatan sulfate, and/or keratan sulfate) biosynthesis alters or disrupts the nature of the glycan (e.g., chondroitin sulfate, dermatan sulfate, and/or keratan sulfate) such that it inhibits glycan (e.g., chondroitin sulfate, dermatan sulfate, and/or keratan sulfate) binding and glycan (e.g., chondroitin sulfate, dermatan sulfate, and/or keratan sulfate) signaling. In some embodiments, modulator of glycan (e.g., chondroitin sulfate, dermatan sulfate, and/or keratan sulfate) biosynthesis alters or disrupts the nature of the glycan (e.g., chondroitin sulfate, dermatan sulfate, and/or keratan sulfate) such that it inhibits the binding, singaling, or a combination thereof of any lectin (including polypeptides) subject to glycan (e.g., chondroitin sulfate, dermatan sulfate, and/or keratan sulfate) binding, signaling or a combination thereof, in the absence of a glycan (e.g., chondroitin sulfate, dermatan sulfate, and/or keratan sulfate) inhibitor. In some embodiments, the lectin is, by way of non-limiting example, a growth factor. In specific embodiments, the growth factor is, by way of non-limiting example, pleiotrophin, midkine, vascular endothelial growth factor (VEGF), fibroblast growth factor (FGF), hepatocyte growth factor, heparin co-factor II or heparin-binding epidermal growth factor (HB-EGF), lamin, nuclear ribonucleoprotein, an antibody, Plasmodium falciparum lectin, annexin 4, annexin 6, PTPsigma, endostatin, or any other chondroitin sulfate binding agent. In certain embodiments, the lectin is 5-D-4 or galectins.

In certain embodiments, the selective modulator of chondroitin sulfate biosynthesis is a small molecule organic compound. In certain instances, selective modulator of chondroitin sulfate biosynthesis utilized herein is not a polypeptide or a carbohydrate. In certain embodiments, the small molecule organic compound has a molecular weight of less than 2,000 g/mol, less than 1,500 g/mol, less than 1,000 g/mol, less than 700 g/mol, or less than 500 g/mol.

Provided in certain embodiments herein is a method of treating cancer or neoplasia comprising administering a therapeutically effective amount of a glycan (e.g., chondroitin sulfate, dermatan sulfate, and/or keratan sulfate) inhibitor to a patient in need thereof. In some embodiments, the chondroitin sulfate inhibitor reduces or inhibits tumor growth, reduces or inhibits angiogenesis, or a combination thereof. In certain embodiments, the glycan (e.g., chondroitin sulfate, dermatan sulfate, and/or keratan sulfate) inhibitor is selective (as compared to other glycans or GAGs) modulator of chondroitin sulfate glycosylation (e.g., inhibits one or more chondroitin sulfate glycosyltransferase), or modulator of chondroitin sulfate sulfation (e.g., inhibits or promotes one or more chondroitin sulfate sulfotransferase). In various embodiments, chondroitin sulfate alters or reduces the function of chondroitin sulfate by one or more of the following non-limiting manners: (1) inhibition of (a) chondroitin sulfate glycosylation; (b) chondroitin sulfate sulfation; and/or (c) chondroitin sulfate phosphorylation; and/or (2) promotion of (a) chondroitin sulfate bond cleavage; (b) bond cleavage of the linker region connecting chondroitin sulfate to a core protein; (c) bond cleavage between chondroitin sulfate and the linker region; (d) sulfation of chondroitin sulfate; and/or (e) chondroitin sulfate phosphorylation in chondroitin sulfate. In specific embodiments, the modulator of chondroitin sulfate biosynthesis inhibits sulfation of chondroitin sulfate. In specific embodiments, the modulator of chondroitin sulfate biosynthesis promotes sulfation of chondroitin sulfate.

In some embodiments, the chondroitin sulfate inhibitor is a selective chondroitin sulfate inhibitor (as compared to the inhibition of the function of other GAGs), e.g., as described herein. In some embodiments, the selective chondroitin sulfate inhibitor is a modulator of (e.g., promotes one or more of, or inhibits one or more of) chondroitin sulfate glycosylation (e.g., modulates a chondroitin sulfate glycosyltransferase), chondroitin sulfate sulfation (e.g., modulates a chondroitin sulfate sulfotransferase), or a combination thereof.

In some embodiments, the chondroitin sulfate inhibitor modulates (e.g., promote or inhibit) glycosyltransferase. In some embodiments, the inhibitor of a chondroitin sulfate glycosyltransferase inhibits the synthesis of the linkage region, the initiation of chondroitin sulfate synthesis, the synthesis of chondroitin sulfate, or a combination thereof. In some embodiments, chondroitin sulfate inhibitors modulate (e.g., promote or inhibit) one or more of a chondroitin sulfate xylosyltransferase, a chondroitin sulfate galactosyltransferase, a chondroitin sulfate glucuronosyltransferase, a chondroitin sulfate N-acetylgalactosaminyl transferase, or combinations thereof. In more specific embodiments, chondroitin sulfate inhibitors selectively modulate (e.g., promote or inhibit) one or more of xylosyltransfarase I, xylosyltransfarase II, galactosyltransferase I, galactosyltransferase II, glucuronosyltransferase I, glucuronosyltransferase II, N-acetylgalactosaminyl transferase I, N-acetylgalactosaminyl transferase II, or a combination thereof.

In certain embodiments, chondroitin sulfate inhibitors that modulate sulfation modulate one or more chondroitin sulfate sulfotransferase. In specific embodiments, the chondroitin sulfate sulfotransferase is, by way of non-limiting example, a modulator (e.g., inhibitor or promoter) of one or more of a chondroitin sulfate O-sulfotransferase. In more specific embodiments, the chondroitin sulfate inhibitor modulates (e.g., inhibits or promotes) a chondroitin sulfate O-sulfotransferase such as, by way of non-limiting example, one or more of a 6-O sulfotransferase (of a galactosaminegroup), a 4-O sulfotransferase (of a galactosaminegroup), a 2-O sulfotransferase (of a uronic acid moiety, e.g., glucuronic acid), or a combination thereof.

In certain embodiments, the effective amount of chondroitin sulfate inhibitor alters or disrupts the nature (e.g., alters or disrupts the acetylation, sulfation, O-sulfation, the 2-O sulfation, the 4-O sulfation, the 6-O sulfation, concentration of chondroitin sulfate, chain length of chondroitin sulfate, or a combination thereof) of chondroitin sulfate compared to endogenous chondroitin sulfate in an amount sufficient to alter or disrupt chondroitin sulfate binding, chondroitin sulfate signaling, or a combination thereof. In specific embodiments, the chondroitin sulfate inhibitor described herein alters or disrupts the nature of the chondroitin sulfate such that it inhibits chondroitin sulfate signaling. In other specific embodiments, the chondroitin sulfate inhibitor described herein alters or disrupts the nature of the chondroitin sulfate such that it inhibits chondroitin sulfate binding. In more specific embodiments, the chondroitin sulfate inhibitor described herein alters or disrupts the nature of the chondroitin sulfate such that it inhibits chondroitin sulfate binding and chondroitin sulfate signaling. In some embodiments, the chondroitin sulfate inhibitor alters or disrupts the nature of the chondroitin sulfate such that it inhibits the binding, singaling, or a combination thereof of any lectin (including polypeptides) subject to chondroitin sulfate binding, signaling or a combination thereof, in the absence of a chondroitin sulfate inhibitor. In some embodiments, the lectin is, by way of non-limiting example, a growth factor. In specific embodiments, the growth factor is, by way of non-limiting example, pleiotrophin, midkine, vascular endothelial growth factor (VEGF), fibroblast growth factor (FGF), hepatocyte growth factor, heparin co-factor II or heparin-binding epidermal growth factor (HB-EGF), lamin, nuclear ribonucleoprotein, an antibody, Plasmodium falciparum lectin, annexin 4, annexin 6, PTPsigma, endostatin, or any other chondroitin sulfate binding agent.

In certain embodiments, chondroitin sulfate inhibitors described herein are small molecule organic compounds. In certain instances, chondroitin sulfate inhibitors utilized herein are not polypeptides or carbohydrates. In some embodiments, a small molecule organic compound has a molecular weight of less than 2,000 g/mol, less than 1,500 g/mol, less than 1,000 g/mol, less than 700 g/mol, or less than 500 g/mol.

Provided in some embodiments herein is a method of treating a lysosomal storage disease comprising administering a therapeutically effective amount of a glycan (e.g., chondroitin sulfate, dermatan sulfate, and/or keratan sulfate) inhibitor to an individual (e.g., a human) in need thereof. In certain embodiments, the glycan (e.g., chondroitin sulfate, dermatan sulfate, and/or keratan sulfate) inhibitor is a selective (as compared to other GAGs) inhibitor of chondroitin sulfate. In some embodiments, the selective glycan (e.g., chondroitin sulfate, dermatan sulfate, and/or keratan sulfate) inhibitor is a selective modulator (e.g., inhibitor or promoter) of chondroitin sulfate glycosylation (e.g., of a chondroitin sulfate glycosyltransferase), or a modulator (e.g., inhibitor or promoter) of chondroitin sulfate sulfation (e.g., of a chondroitin sulfate sulfotransferase).

In specific embodiments, the lysosomal storage disease is, by way of non-limiting example, mucopolysaccharidosis (MPS). In more specific embodiments, the MPS is, by way of non-limiting example, MPS IV, MPS VI or MPS VII.

In various embodiments, the chondroitin sulfate inhibitor alters or disrupts the function of chondroitin sulfate by one or more of the following non-limiting manners: (1) inhibition of (a) chondroitin sulfate glycosylation; (b) chondroitin sulfate sulfation; and/or (d) chondroitin sulfate phosphorylation; and/or (2) promotion of (a) chondroitin sulfate bond cleavage; (b) bond cleavage of the linker region connecting chondroitin sulfate to a core protein; (c) bond cleavage between chondroitin sulfate and the linker region; (d) sulfation of chondroitin sulfate; and/or (g) chondroitin sulfate phosphorylation.

In various embodiments, the chondroitin sulfate inhibitor alters or disrupts the function of chondroitin sulfate by one or more of the following non-limiting manners: (1) inhibition of (a) chondroitin sulfate glycosylation; (b) chondroitin sulfate sulfation; and/or (c) chondroitin sulfate phosphorylation; and/or (2) promotion of (a) chondroitin sulfate bond cleavage; (b) bond cleavage of the linker region connecting chondroitin sulfate to a core protein; (c) bond cleavage between chondroitin sulfate and the linker region; (d) sulfation of chondroitin sulfate; and/or (e) chondroitin sulfate phosphorylation.

In some embodiments, the chondroitin sulfate inhibitor is a selective chondroitin sulfate inhibitor (as compared to the inhibition of the function of other GAGs), e.g., as described herein. In some embodiments, the selective chondroitin sulfate inhibitor is a modulator of (e.g., promotes one or more of, or inhibits one or more of) chondroitin sulfate glycosylation (e.g., modulates a chondroitin sulfate glycosyltransferase), chondroitin sulfate sulfation (e.g., modulates a chondroitin sulfate sulfotransferase), or a combination thereof.

In some embodiments, the chondroitin sulfate inhibitor modulates (e.g., promote or inhibit) glycosyltransferase. In some embodiments, the inhibitor of a chondroitin sulfate glycosyltransferase inhibits the synthesis of the linkage region, the initiation of chondroitin sulfate synthesis, the synthesis of chondroitin sulfate, or a combination thereof. In some embodiments, chondroitin sulfate inhibitors modulate (e.g., promote or inhibit) one or more of a chondroitin sulfate xylosyltransferase, a chondroitin sulfate galactosyltransferase, a chondroitin sulfate glucuronosyltransferase, a chondroitin sulfate N-acetylgalactosaminyl transferase, or combinations thereof. In more specific embodiments, chondroitin sulfate inhibitors selectively modulate (e.g., promote or inhibit) one or more of xylosyltransfarase I, xylosyltransfarase II, galactosyltransferase I, galactosyltransferase II, glucuronosyltransferase I, glucuronosyltransferase II, N-acetylgalactosaminyl transferase I, N-acetylgalactosaminyl transferase II, or a combination thereof.

In certain embodiments, chondroitin sulfate inhibitors that modulate sulfation modulate one or more chondroitin sulfate sulfotransferase. In specific embodiments, the chondroitin sulfate sulfotransferase is, by way of non-limiting example, a modulator (e.g., inhibitor or promoter) of one or more of a chondroitin sulfate O-sulfotransferase. In more specific embodiments, the chondroitin sulfate inhibitor modulates (e.g., inhibits or promotes) a chondroitin sulfate O-sulfotransferase such as, by way of non-limiting example, one or more of a 6-O sulfotransferase (of a galactosaminyl group), a 4-O sulfotransferase (of a galactosaminyl group), a 2-O sulfotransferase (of a uronic acid moiety, e.g., glucuronic acid), a 6-O sulfotransferase of galactose), a 4-O sulfotransferase of galactose or a combination thereof.

In certain embodiments, the effective amount of chondroitin sulfate inhibitor alters or disrupts the nature (e.g., alters or disrupts the acetylation, sulfation, O-sulfation, the 2-O sulfation, the 4-O sulfation, the 6-O sulfation, concentration of chondroitin sulfate, chain length of chondroitin sulfate, phosphorylation, or a combination thereof) of chondroitin sulfate compared to endogenous chondroitin sulfate in an amount sufficient to alter or disrupt chondroitin sulfate binding, chondroitin sulfate signaling, or a combination thereof. In specific embodiments, the chondroitin sulfate inhibitor described herein alters or disrupts the nature of the chondroitin sulfate such that it inhibits chondroitin sulfate signaling. In other specific embodiments, the chondroitin sulfate inhibitor described herein alters or disrupts the nature of the chondroitin sulfate such that it inhibits chondroitin sulfate binding. In more specific embodiments, the chondroitin sulfate inhibitor described herein alters or disrupts the nature of the chondroitin sulfate such that it inhibits chondroitin sulfate binding and chondroitin sulfate signaling. In some embodiments, the chondroitin sulfate inhibitor alters or disrupts the nature of the chondroitin sulfate such that it inhibits the binding, singaling, or a combination thereof of any lectin (including polypeptides) subject to chondroitin sulfate binding, signaling or a combination thereof, in the absence of a chondroitin sulfate inhibitor. In some embodiments, the lectin is, by way of non-limiting example, a growth factor. In specific embodiments, the growth factor is, by way of non-limiting example, pleiotrophin, midkine, vascular endothelial growth factor (VEGF), fibroblast growth factor (FGF), hepatocyte growth factor, heparin co-factor II or heparin-binding epidermal growth factor (HB-EGF), lamin, nuclear ribonucleoprotein, an antibody, Plasmodium falciparum lectin, annexin 4, annexin 6, PTPsigma, endostatin, or any other chondroitin sulfate binding agent.

In certain embodiments, chondroitin sulfate inhibitors described herein are small molecule organic compounds. In certain instances, chondroitin sulfate inhibitors utilized herein are not polypeptides or carbohydrates. In some embodiments, a small molecule organic compound has a molecular weight of less than 2,000 g/mol, less than 1,500 g/mol, less than 1,000 g/mol, less than 700 g/mol or less than 500 g/mol.

Provided in some embodiments herein is a method of treating a inflammatory disease comprising administering a therapeutically effective amount of a glycan (e.g., chondroitin sulfate, dermatan sulfate, and/or keratan sulfate) inhibitor to an individual (e.g., a human) in need thereof. In certain embodiments, the chondroitin sulfate inhibitor is a selective (as compared to other GAGs) inhibitor of chondroitin sulfate. In some embodiments, the selective chondroitin sulfate inhibitor is a selective modulator (e.g., inhibitor or promoter) of chondroitin sulfate glycosylation (e.g., of a chondroitin sulfate glycosyltransferase), or a modulator (e.g., inhibitor or promoter) of chondroitin sulfate sulfation (e.g., of a chondroitin sulfate sulfotransferase).

In specific embodiments, the inflammatory disease is, by way of non-limiting example, osteoarthritis.

In various embodiments, the chondroitin sulfate inhibitor alters or disrupts the function of chondroitin sulfate by one or more of the following non-limiting manners: (1) inhibition of (a) chondroitin sulfate glycosylation; (b) chondroitin sulfate sulfation; and/or (d) chondroitin sulfate phosphorylation; and/or (2) promotion of (a) chondroitin sulfate bond cleavage; (b) bond cleavage of the linker region connecting chondroitin sulfate to a core protein; (c) bond cleavage between chondroitin sulfate and the linker region; (d) sulfation of chondroitin sulfate; and/or (g) chondroitin sulfate phosphorylation.

In various embodiments, the chondroitin sulfate inhibitor alters or disrupts the function of chondroitin sulfate by one or more of the following non-limiting manners: (1) inhibition of (a) chondroitin sulfate glycosylation; (b) chondroitin sulfate sulfation; and/or (c) chondroitin sulfate phosphorylation; and/or (2) promotion of (a) chondroitin sulfate bond cleavage; (b) bond cleavage of the linker region connecting chondroitin sulfate to a core protein; (c) bond cleavage between chondroitin sulfate and the linker region; (d) sulfation of chondroitin sulfate; and/or (e) chondroitin sulfate phosphorylation.

In some embodiments, the chondroitin sulfate inhibitor is a selective chondroitin sulfate inhibitor (as compared to the inhibition of the function of other GAGs), e.g., as described herein. In some embodiments, the selective chondroitin sulfate inhibitor is a modulator of (e.g., promotes one or more of, or inhibits one or more of) chondroitin sulfate glycosylation (e.g., modulates a chondroitin sulfate glycosyltransferase), chondroitin sulfate sulfation (e.g., modulates a chondroitin sulfate sulfotransferase), or a combination thereof.

In some embodiments, the glycan (e.g., chondroitin sulfate, dermatan sulfate, and/or keratan sulfate) inhibitor modulates (e.g., promote or inhibit) glycosyltransferase. In some embodiments, the inhibitor of a glycan (e.g., chondroitin sulfate, dermatan sulfate, and/or keratan sulfate) glycosyltransferase inhibits the synthesis of the linkage region, the initiation of glycan (e.g., chondroitin sulfate, dermatan sulfate, and/or keratan sulfate) synthesis, the synthesis of glycan (e.g., chondroitin sulfate, dermatan sulfate, and/or keratan sulfate), or a combination thereof. In some embodiments, glycan (e.g., chondroitin sulfate, dermatan sulfate, and/or keratan sulfate) inhibitors modulate (e.g., promote or inhibit) one or more of a xylosyltransferase, a galactosyltransferase, a glucuronosyltransferase, a N-acetylgalactosaminyl transferase, or combinations thereof. In more specific embodiments, glycan (e.g., chondroitin sulfate, dermatan sulfate, and/or keratan sulfate) inhibitors selectively modulate (e.g., promote or inhibit) one or more of xylosyltransfarase I, xylosyltransfarase II, galactosyltransferase I, galactosyltransferase II, glucuronosyltransferase I, glucuronosyltransferase II, N-acetylgalactosaminyl transferase I, N-acetylgalactosaminyl transferase II, or a combination thereof.

In certain embodiments, chondroitin sulfate inhibitors that modulate sulfation modulate one or more chondroitin sulfate sulfotransferase. In specific embodiments, the chondroitin sulfate sulfotransferase is, by way of non-limiting example, a modulator (e.g., inhibitor or promoter) of one or more of a chondroitin sulfate O-sulfotransferase. In more specific embodiments, the chondroitin sulfate inhibitor modulates (e.g., inhibits or promotes) a chondroitin sulfate O-sulfotransferase such as, by way of non-limiting example, one or more of a 6-O sulfotransferase (of a galactosaminyl group), a 4-O sulfotransferase (of a galactosaminyl group), a 2-O sulfotransferase (of a uronic acid moiety, e.g., glucuronic acid), a 6-O sulfotransferase of galactose), a 4-O sulfotransferase of galactose or a combination thereof.

In certain embodiments, the effective amount of chondroitin sulfate inhibitor alters or disrupts the nature (e.g., alters or disrupts the acetylation, sulfation, O-sulfation, the 2-O sulfation, the 4-O sulfation, the 6-O sulfation, concentration of chondroitin sulfate, chain length of chondroitin sulfate, phosphorylation, or a combination thereof) of chondroitin sulfate compared to endogenous chondroitin sulfate in an amount sufficient to alter or disrupt chondroitin sulfate binding, chondroitin sulfate signaling, or a combination thereof. In specific embodiments, the chondroitin sulfate inhibitor described herein alters or disrupts the nature of the chondroitin sulfate such that it inhibits chondroitin sulfate signaling. In other specific embodiments, the chondroitin sulfate inhibitor described herein alters or disrupts the nature of the chondroitin sulfate such that it inhibits chondroitin sulfate binding. In more specific embodiments, the chondroitin sulfate inhibitor described herein alters or disrupts the nature of the chondroitin sulfate such that it inhibits chondroitin sulfate binding and chondroitin sulfate signaling. In some embodiments, the chondroitin sulfate inhibitor alters or disrupts the nature of the chondroitin sulfate such that it inhibits the binding, singaling, or a combination thereof of any lectin (including polypeptides) subject to chondroitin sulfate binding, signaling or a combination thereof, in the absence of a chondroitin sulfate inhibitor. In some embodiments, the lectin is, by way of non-limiting example, a growth factor. In specific embodiments, the growth factor is, by way of non-limiting example, pleiotrophin, midkine, vascular endothelial growth factor (VEGF), fibroblast growth factor (FGF), hepatocyte growth factor, heparin co-factor II or heparin-binding epidermal growth factor (HB-EGF) lamin, nuclear ribonucleoprotein, an antibody, Plasmodium falciparum lectin, annexin 4, annexin 6, PTPsigma, endostatin, or any other chondroitin sulfate binding agent.

In certain embodiments, chondroitin sulfate inhibitors described herein are small molecule organic compounds. In certain instances, chondroitin sulfate inhibitors utilized herein are not polypeptides or carbohydrates. In some embodiments, a small molecule organic compound has a molecular weight of less than 2,000 g/mol, less than 1,500 g/mol, less than 1,000 g/mol, 700 g/mol, or less than 500 g/mol.

Provided in some embodiments herein is a method of treating a injury to the central nervous system (CNS) comprising administering a therapeutically effective amount of a glycan (e.g., chondroitin sulfate, dermatan sulfate, and/or keratan sulfate) inhibitor to an individual (e.g., a human) in need thereof. In certain embodiments, the glycan (e.g., chondroitin sulfate, dermatan sulfate, and/or keratan sulfate) inhibitor is a selective (as compared to other GAGs) inhibitor of chondroitin sulfate. In some embodiments, the selective chondroitin sulfate inhibitor is a selective modulator (e.g., inhibitor or promoter) of chondroitin sulfate glycosylation (e.g., of a chondroitin sulfate glycosyltransferase), or a modulator (e.g., inhibitor or promoter) of chondroitin sulfate sulfation (e.g., of a chondroitin sulfate sulfotransferase). In certain embodiments, the injury to the central nervous system (CNS) is a head injury, brain injury, spinal cord injury, or any combination thereof.

In various embodiments, the chondroitin sulfate inhibitor alters or disrupts the function of chondroitin sulfate by one or more of the following non-limiting manners: (1) inhibition of (a) chondroitin sulfate glycosylation; (b) chondroitin sulfate sulfation; and/or (d) chondroitin sulfate phosphorylation; and/or (2) promotion of (a) chondroitin sulfate bond cleavage; (b) bond cleavage of the linker region connecting chondroitin sulfate to a core protein; (c) bond cleavage between chondroitin sulfate and the linker region; (d) sulfation of chondroitin sulfate; and/or (g) chondroitin sulfate phosphorylation.

In various embodiments, the chondroitin sulfate inhibitor alters or disrupts the function of chondroitin sulfate by one or more of the following non-limiting manners: (1) inhibition of (a) chondroitin sulfate glycosylation; (b) chondroitin sulfate sulfation; and/or (c) chondroitin sulfate phosphorylation; and/or (2) promotion of (a) chondroitin sulfate bond cleavage; (b) bond cleavage of the linker region connecting chondroitin sulfate to a core protein; (c) bond cleavage between chondroitin sulfate and the linker region; (d) sulfation of chondroitin sulfate; and/or (e) chondroitin sulfate phosphorylation.

In some embodiments, the chondroitin sulfate inhibitor is a selective chondroitin sulfate inhibitor (as compared to the inhibition of the function of other GAGs), e.g., as described herein. In some embodiments, the selective chondroitin sulfate inhibitor is a modulator of (e.g., promotes one or more of, or inhibits one or more of) chondroitin sulfate glycosylation (e.g., modulates a chondroitin sulfate glycosyltransferase), chondroitin sulfate sulfation (e.g., modulates a chondroitin sulfate sulfotransferase), or a combination thereof.

In some embodiments, the chondroitin sulfate inhibitor modulates (e.g., promote or inhibit) glycosyltransferase. In some embodiments, the inhibitor of a chondroitin sulfate glycosyltransferase inhibits the synthesis of the linkage region, the initiation of chondroitin sulfate synthesis, the synthesis of chondroitin sulfate, or a combination thereof. In some embodiments, chondroitin sulfate inhibitors modulate (e.g., promote or inhibit) one or more of a chondroitin sulfate xylosyltransferase, a chondroitin sulfate galactosyltransferase, a chondroitin sulfate glucuronosyltransferase, a chondroitin sulfate N-acetylgalactosaminyl transferase, or combinations thereof. In more specific embodiments, chondroitin sulfate inhibitors selectively modulate (e.g., promote or inhibit) one or more of xylosyltransfarase I, xylosyltransfarase II, galactosyltransferase I, galactosyltransferase II, glucuronosyltransferase I, glucuronosyltransferase II, N-acetylgalactosaminyl transferase I, N-acetylgalactosaminyl transferase II, or a combination thereof.

In certain embodiments, glycan (e.g., chondroitin sulfate, dermatan sulfate, and/or keratan sulfate) inhibitors that modulate sulfation modulate one or more glycan (e.g., chondroitin sulfate, dermatan sulfate, and/or keratan sulfate) sulfotransferase. In specific embodiments, the glycan (e.g., chondroitin sulfate, dermatan sulfate, and/or keratan sulfate) sulfotransferase is, by way of non-limiting example, a modulator (e.g., inhibitor or promoter) of one or more of a chondroitin sulfate O-sulfotransferase. In more specific embodiments, the glycan (e.g., chondroitin sulfate, dermatan sulfate, and/or keratan sulfate) inhibitor modulates (e.g., inhibits or promotes) a glycan (e.g., chondroitin sulfate, dermatan sulfate, and/or keratan sulfate) O-sulfotransferase such as, by way of non-limiting example, one or more of a 6-O sulfotransferase (of a galactosaminyl group), a 4-O sulfotransferase (of a galactosaminyl group), a 2-O sulfotransferase (of a uronic acid moiety, e.g., glucuronic acid), a 6-O sulfotransferase of galactose), a 4-O sulfotransferase of galactose or a combination thereof.

In certain embodiments, the effective amount of glycan (e.g., chondroitin sulfate, dermatan sulfate, and/or keratan sulfate) inhibitor alters or disrupts the nature (e.g., alters or disrupts the acetylation, sulfation, O-sulfation, the 2-O sulfation, the 4-O sulfation, the 6-O sulfation, concentration of glycan (e.g., chondroitin sulfate, dermatan sulfate, and/or keratan sulfate), chain length of glycan (e.g., chondroitin sulfate, dermatan sulfate, and/or keratan sulfate), phosphorylation, or a combination thereof) of glycan (e.g., chondroitin sulfate, dermatan sulfate, and/or keratan sulfate) compared to endogenous glycan (e.g., chondroitin sulfate, dermatan sulfate, and/or keratan sulfate) in an amount sufficient to alter or disrupt glycan (e.g., chondroitin sulfate, dermatan sulfate, and/or keratan sulfate) binding, glycan (e.g., chondroitin sulfate, dermatan sulfate, and/or keratan sulfate) signaling, or a combination thereof. In specific embodiments, the glycan (e.g., chondroitin sulfate, dermatan sulfate, and/or keratan sulfate) inhibitor described herein alters or disrupts the nature of the glycan (e.g., chondroitin sulfate, dermatan sulfate, and/or keratan sulfate) such that it inhibits glycan (e.g., chondroitin sulfate, dermatan sulfate, and/or keratan sulfate) signaling. In other specific embodiments, the glycan (e.g., chondroitin sulfate, dermatan sulfate, and/or keratan sulfate) inhibitor described herein alters or disrupts the nature of the glycan (e.g., chondroitin sulfate, dermatan sulfate, and/or keratan sulfate) such that it inhibits glycan (e.g., chondroitin sulfate, dermatan sulfate, and/or keratan sulfate) binding. In more specific embodiments, the glycan (e.g., chondroitin sulfate, dermatan sulfate, and/or keratan sulfate) inhibitor described herein alters or disrupts the nature of the glycan (e.g., chondroitin sulfate, dermatan sulfate, and/or keratan sulfate) such that it inhibits glycan (e.g., chondroitin sulfate, dermatan sulfate, and/or keratan sulfate) binding and glycan (e.g., chondroitin sulfate, dermatan sulfate, and/or keratan sulfate) signaling. In some embodiments, the glycan (e.g., chondroitin sulfate, dermatan sulfate, and/or keratan sulfate) inhibitor alters or disrupts the nature of the glycan (e.g., chondroitin sulfate, dermatan sulfate, and/or keratan sulfate) such that it inhibits the binding, signaling, or a combination thereof of any lectin (including polypeptides) subject to glycan (e.g., chondroitin sulfate, dermatan sulfate, and/or keratan sulfate) binding, signaling or a combination thereof, in the absence of a glycan (e.g., chondroitin sulfate, dermatan sulfate, and/or keratan sulfate) inhibitor. In some embodiments, the lectin is, by way of non-limiting example, a growth factor. In specific embodiments, the growth factor is, by way of non-limiting example, pleiotrophin, midkine, vascular endothelial growth factor (VEGF), fibroblast growth factor (FGF), hepatocyte growth factor, heparin co-factor II or heparin-binding epidermal growth factor (HB-EGF) lamin, nuclear ribonucleoprotein, an antibody, Plasmodium falciparum lectin, annexin 4, annexin 6, PTPsigma, endostatin, or any other chondroitin sulfate binding agent. In some embodiments, the lectin is 5-D-4, or galectins.

In certain embodiments, glycan (e.g., chondroitin sulfate, dermatan sulfate, and/or keratan sulfate) inhibitors described herein are small molecule organic compounds. In certain instances, glycan (e.g., chondroitin sulfate, dermatan sulfate, and/or keratan sulfate) inhibitors utilized herein are not polypeptides or carbohydrates. In some embodiments, a small molecule organic compound has a molecular weight of less than 2,000 g/mol, less than 1,500 g/mol, less than 1,000 g/mol, less than 700 g/mol, or less than 500 g/mol.

In various embodiments, any method described herein provides delivery (e.g., systemic delivery, topical delivery, or localized delivery) of any agent or combination of agents described herein to an individual or cell within an individual by oral administration, nasal administration, pulmonary administration, ocular administration, topical administration, intrathecal administration, intraperitoneal administration, intravenous administration, intraarterial administration, intracardiac administration, intraosseous administration, intrasynovial administration, intracutaneous administration, subcutaneous administration, intramuscular administration, and intradermal administration, intracranial administration, intralesional administration, transdermal administration, sublingual administration, buccal administration, intracerebral administration, intracerebroventricular administration, intracisternal administration, peridural (epidural) administration and/or intratumoral administration.

In specific embodiments, methods of treating spinal cord injury in an individual comprise administering any agent or combination of agents described herein intrathecally, systemically, and/or topically. In some embodiments, a method of treating spinal cord injury in an individual comprises administering any agent or combination of agents described herein at an appropriate time, including, by way of non-limiting example, immediately after injury, within one day of injury, within two days of injury, within three days of injury, within one week of injury, within two weeks of injury, within 30 days of injury, within 60 days of injury, or the like. In specific embodiments, any therapy for the threatment of spinal cord injury may be combined with any other suitable therapy, including, by way of non-limiting example, stem cell therapy.

### Screening Processes

Provided in some embodiments is a process for identifying a compound that modulates galactosamine and glucuronic acid containing glycans (e.g., chondroitin sulfate and/or dermatan sulfate) biosynthesis comprising: contacting a mammalian cell with the compound in combination with a labeled probe that binds a galactosamine and glucuronic acid containing glycan;
a. incubating the mammalian cell, compound and labeled probe;
b. collecting the labeled probe that is glycan-bound; and
c. detecting or measuring the amount of labeled probe that is glycan-bound.

Provided in some embodiments is a process for identifying a compound that modulates galactose and glucosamine containing glycans (e.g., keratan sulfate) biosynthesis comprising:
a. contacting a mammalian cell with the compound in combination with a labeled probe that binds a galactose and glucosamine containing glycan;
b. incubating the mammalian cell, compound and labeled probe;
c. collecting the labeled probe that is glycan-bound; and
d. detecting or measuring the amount of labeled probe that is glycan-bound.

In more specific embodiments, provided herein is a process for identifying a compound that selectively modulates galactosamine and uronic acid containing glycan (e.g., chondroitin sulfate and/or dermatan sulfate) biosynthesis comprising:
a. contacting a mammalian cell with the compound
b. contacting the mammalian cell and compound combination with a first labeled probe and a second labeled probe, wherein the first labeled probe binds chondroitin sulfate and/or dermatan sulfate and the second labeled probe binds at least one glycan (e.g., a GAG, a sulfated GAG, a non-GAG glycan, or the like) other than one or more of the galactosamine and uronic acid containing glycans (e.g., chondroitin sulfate and/or dermatan sulfate);
c. incubating the mammalian cell, compound, the first labeled probe, and the second labeled probe;
d. collecting the first labeled probe that is glycan-bound;
e. collecting the second labeled probe that is bound to at least one glycan (e.g., a GAG, a sulfated GAG, a non-GAG glycan, or the like) other than the one or more galactosamine and uronic acid containing glycan (e.g., chondroitin sulfate and/or dermatan sulfate);
f. detecting or measuring the amount of first labeled probe that is glycan-bound; and
g. detecting or measuring the amount of the second labeled probe that is glycan-bound (e.g., a GAG, a sulfated GAG, anon-GAG glycan, or the like).

In some embodiments, the process further comprises comparing the amount of first labeled probe that is glycan-bound to a control. In some embodiments, the process further comprises comparing the amount of first labeled probe that is glycan-bound to a control obtained or obtainable by contacting a similar or identical mammalian cell with the first labeled probe (e.g., in the absence of the compound), incubating the cell and the first labeled probe, collecting the first labeled probe that is glycan bound, and detecting the amount of first labeled probe that is glycan-bound. Similarly, some embodiments of any process described herein the process further comprises comparing the amount of second labeled probe that is glycan-bound to a control. In some embodiments, the process further comprises comparing the amount of second labeled probe that is glycan-bound to a control obtained or obtainable by contacting a similar or identical mammalian cell with the second labeled probe (e.g., in the absence of the compound), incubating the cell and the second labeled probe, collecting the second labeled probe that is glycan bound, and detecting the amount of second labeled probe that is glycan-bound. In specific embodiments, the amount of bound first labeled probe is compared to a first control and the amount of bound second labeled probe is compared to a second control.

Similarly, in some embodiments provided herein is a process for identifying compounds that selectively modulate galactosamine and uronic acid containing glycan (e.g., chondroitin sulfate and/or dermatan sulfate) biosynthesis comprising:
a. contacting a first mammalian cell (or population of cells) with the compound
b. contacting the first mamallian cell and compound combination with a first labeled probe, wherein the first labeled probe binds galactosamine and uronic acid containing glycan;
c. incubating the first mammalian cell, compound, and the first labeled probe;
d. collecting the first labeled probe that is glycan-bound;
e. detecting or measuring the amount of first labeled probe glycan-bound;
f. contacting a second mammalian cell with the compound, wherein the second mammalian cell is of the same type as the first mammalian cell;
g. contacting the second mammalian cell and compound combination with a second labeled probe, wherein the second labeled probe binds at least one glycan (e.g., a GAG, a sulfated GAG, a non-GAG glycan, or the like) other than one or more of the galactosamine and uronic acid containing glycans (e.g., chondroitin sulfate and/or dermatan sulfate);
h. collecting the second labeled probe that is bound that is glycan-bound (e.g., a GAG, a sulfated GAG, a non-GAG glycan, or the like); and
i. detecting or measuring the amount of the second labeled probe that is glycan-bound (e.g., a GAG, a sulfated GAG, a non-GAG glycan, or the like).

In some embodiments, the process further comprises comparing the amount of first labeled probe bound to galactosamine and uronic acid containing glycan (e.g., chondroitin sulfate and/or dermatan sulfate) to the amount of the second labeled probe bound to at least one glycan other than one or more of the galactosamine and uronic acid containing glycans (e.g., to determine a ratio of the amount of first labeled probe bound to the amount of second labeled probe bound under substantially similar conditions).

In some embodiments, any process described herein further comprises comparing a labeled probe amount (e.g., a first or second labeled probe) to a control. In certain instances, the control is the amount of glycan bound labeled probe measured in a cell (or population of cells) treated in a manner similar or identical to that described above, with the exception that the cell (or population of cells) is not treated with the compound.

In certain embodiments, a label utilized in any process described herein is any suitable label such as, by way of non-limiting example, a fluorecent label, a dye, a radiolabel, or the like. In some embodiments, the labeled probe comprises a biotinyl moiety and the process further comprises tagging the labeled probe with streptavidin-Cy5-PE. In certain embodiments, the first probe is any chondroitin and/or dermatan sulfate binding lectin, e.g., a growth factor. In specific embodiments, the growth factor is, by way of non-limiting example, FGF (e.g., FGF-2, FGF-16), HB-EGF, VEGF, hepatocyte growth factor, heparin co-factor II, pleiotrophin or midkine, lamin, nuclear ribonucleoprotein, an antibody, Plasmodium falciparum lectin, annexin 4, annexin 6, PTPsigma, endostatin, or any other chondroitin sulfate binding agent. In certain embodiments, the first probe is any labeled keratan sulfate binding lectin, e.g., 5-D-4, or galectins. In various embodiments, the amount of bound labeled probes are detected in any suitable manner, e.g., with a fluorimeter, a radiation detector, or the like.

In certain embodiments, the first and second probes are labeled in a manner so as to be independently detectable. In some embodiments, the first and second probes are contacted to the cells separately (i.e., to different cells of the same type) and independently analyzed. In some embodiments, the at least one glycan (e.g., a GAG, a sulfated GAG, a non-GAG glycan, or the like) other than chondroitin sulfate is, by way of non-limiting example, chondroitin sulfate, O-linked glycans, N-linked glycans, gangliosides, or the like. Furthermore, in some embodiments, a third labeled probe that binds at least one glycan (e.g., a GAG, a sulfated GAG, a non-GAG glycan, or the like) not bound by the first or second labeled probe is also utilized. Additional labeled probes are also optionally utilized.

Second and additional labeled probes include any labeled compound or labeled lectin suitable (e.g., a labeled compound or lectin that binds a non-chondroitin sulfate GAG, a non-chondroitin sulfate glycan, a non-sulfated GAG, a non-GAG glycan, an O-linked glycan, an N-linked glycan, a ganglioside, chondroitin sulfate, dermatan sulfate, keratan sulfate, and/or hyaluronan). In some embodiments, labeled probes included labeled forms of one or more of, by way of non-limiting example, Wheat Germ Agglutinin (WGA) from Triticum vulgaris (as a probe for binding N-linked and O-linked glycans with terminal GlcNAc residues and clustered sialic acid residues); Phaseolus Vulgaris Aggutinin (PHA) from Phaseolus vulgaris (as a probe for binding N-linked glycans); Cholera Toxin B-subunit (CTB) from Vibrio cholera (as a probe for binding sialic acid modified glycolipids); Concanavalin A (ConA) from Canavalia ensiformis (as a probe for binding mannose residues in N-linked glycans); and/or Jacalin from Artocarpus integrifolia (as a probe for binding O-linked glycans). In specific embodiments, labeled forms of each of Wheat Germ Agglutinin (WGA) from Triticum vulgaris (as a probe for binding N-linked and O-linked glycans with terminal GlcNAc residues and clustered sialic acid residues); Phaseolus Vulgaris Aggutinin (PHA) from Phaseolus vulgaris (as a probe for binding N-linked glycans); and Cholera Toxin B-subunit (CTB) from Vibrio cholera (as a probe for binding sialic acid modified glycolipids) are utilized.

Contact with first, second and additional labeled probes occurs in parallel, concurrently, or sequentially. In certain embodiments, contact the compounds and multiple probes allows identification of selective chondroitin sulfate inhibitors.

In some embodiments, the mammalian cell (e.g., human cell) is selected from any suitable mammalian cell. In specific embodiments, the mammalian cell is, by way of non-limiting example, a human cancer cell (e.g., human cervical cancer cell (HeLa)), a human ovarian cancer cell (SKOV), a human lung cancer cell (Hal8), a human meduloblastoma cancer cell (DAOY), a Chinese Hamster Ovary (CHO) cell, or a human primary cell. In certain embodiments, included herein are processes wherein the cell includes a plurality (e.g., 2, 3, 4 or all) of a human cancer cell (e.g., human cervical cancer cell (HeLa)), a human ovarian cancer cell (SKOV), a human lung cancer cell (Hal8), a human meduloblastoma cancer cell (DAOY), a human melanoma cell (SK-MEL), and/or a Chinese Hamster Ovary (CHO) cell. Contact with such cells optionally occurs in parallel, concurrently, or sequentially. In certain embodiments, contact of multiple cells identification of chondroitin sulfate inhibitors (e.g., selective chondroitin sulfate inhibitors) that inhibit chondroitin sulfate biosynthesis in multiple cell lines. In some instances, utilization of a plurality of cell lines allows the elimination or minimization of false positives in identifying chondroitin sulfate inhibitors.

Thus, in some embodiments, any process described herein comprises contacting the compound to a first cell (type), contacting the compound to a second cell (type), and, optionally, contacting the compound to additional cells (types), and repeating the process described for each of the first, second and any additional cell types utilized (e.g., to determine if a chondroitin sulfate inhibitor is selective for multiple cell lines or to determine which types of cell lines that the chondroitin sulfate inhibitor selectively targets). Furthermore, in such embodiments, the process further comprises comparing the amount of labeled probe (or the amount of first, second or any additional labeled probe) that is bound in each type of cell (e.g., to determine selectively of inhibiting chondroitin sulfate biosynthesis compared to the biosynthesis of other types of glycans).

In some embodiments, once a compound that modulates chondroitin sulfate biosynthesis is determined by the process described, a similar process is optionally utilized to determine whether or not the compound selectively modulates chondroitin sulfate biosynthesis. Specifically, selectivity of a compound that modulates chondroitin sulfate biosynthesis is determined by utilizing a similar process as described for determining whether or not the compound modulates chondroitin sulfate biosynthesis, e.g., by:
a. contacting a mammalian cell with the compound in combination with a labeled probe that binds one or more non-chondroitin sulfate glycan (e.g., another GAG or other class of glycan);
b. incubating the mammalian cell, compound and labeled probe;
c. collecting the labeled probe that is bound to non-chondroitin sulfate glycan (e.g., another GAG or other class of glycan); and
d. detecting or measuring the amount of labeled probe bound to non-chondroitin sulfate glycan (e.g., another GAG or other class of glycan).

In various embodiments, this process is repeated for any number of non-chondroitin sulfate glycans (e.g., another GAG or other class of glycan). In some embodiments, the non-chondroitin sulfate glycans are, by way of non-limiting example, heparan sulfate, O-linked glycans, N-linked glycans, gangliosides, or the like.

Furthermore, provided in some embodiments herein is a process for identifying a compound that modulates chondroitin sulfate (and/or dermatan sulfate) biosynthesis or a process for identifying the effect of a compound on chondroitin sulfate (and/or dermatan sulfate) biosynthesis comprising:
a. collecting chondroitin sulfate (and/or dermatan sulfate) from a first mammalian cell of a selected type, wherein the chondroitin sulfate (and/or dermatan sulfate) is a sulfated oligosaccharide comprising galactosaminyl groups and uronic acid groups;
b. cleaving the chondroitin sulfate (and/or dermatan sulfate) into a plurality of disaccharide component parts;
c. measuring:
   i. the amount of chondroitin sulfate (and/or dermatan sulfate) disaccharides produced by the first mammalian cell,
   ii. the amount of 6-OH sulfation of the galactosaminyl groups, the 4-OH sulfation of the galactosaminyl groups, the 2-OH sulfation of the uronic acid groups, or a combination thereof of the chondroitin sulfate (and/or dermatan sulfate),
   iii. the pattern of sulfation (domain organization); or
   iv. a combination thereof; and
d. contacting and incubating a second mammalian cell of the selected type with the compound;
e. collecting modified chondroitin sulfate (and/or dermatan sulfate) from the second mammalian cell, wherein the modified chondroitin sulfate (and/or dermatan sulfate) is sulfated oligosaccharide comprising galactosaminyl groups and uronic acid groups;
f. cleaving the modified chondroitin sulfate (and/or dermatan sulfate) into a plurality of disaccharide component parts;
g. measuring:
   i. the amount of chondroitin sulfate (and/or dermatan sulfate) disaccharides produced by the second mammalian cell,
   ii. the amount of 6-OH sulfation of the galactosaminyl groups, the 4-OH sulfation of the galactosaminyl groups, the 2-OH sulfation of the uronic acid groups, or a combination thereof of the modified chondroitin sulfate (and/or dermatan sulfate),
   iii. the pattern of sulfation (domain organization); or
   iv. a combination thereof; and
h. comparing:
   i. the amounts of chondroitin sulfate (and/or dermatan sulfate) disaccharides produced by the first and second mammalian cells,
   ii. the amounts of 6-OH sulfation of the galactoseaminyl groups, the 4-OH sulfation of the galactoseaminyl groups, the 2-OH sulfation of the uronic acid groups, pattern of sulfation, or a combination thereof of the chondroitin sulfate (and/or dermatan sulfate) and the modified chondroitin sulfate (and/or dermatan sulfate), or
   iii. a combination thereof.

Furthermore, provided in some embodiments herein is a process for identifying a compound that modulates keratan sulfate biosynthesis or a process for identifying the effect of a compound on keratan sulfate biosynthesis comprising:
a. collecting keratan sulfate from a first mammalian cell of a selected type, wherein the keratan sulfate is a sulfated oligosaccharide comprising glucosaminyl groups and galactose groups;
b. cleaving the keratan sulfate into a plurality of disaccharide component parts;
c. measuring:
   i. the amount of keratan sulfate disaccharides produced by the first mammalian cell,
   ii. the amount of 6-OH sulfation of the glucosaminyl groups, the 6-OH sulfation of the galactose groups, or a combination thereof of the keratan sulfate,
   iii. the pattern of sulfation (domain organization); or
   iv. a combination thereof; and
d. contacting and incubating a second mammalian cell of the selected type with the compound;
e. collecting modified keratan sulfate from the second mammalian cell, wherein the modified keratan sulfate is a sulfated oligosaccharide comprising glucosaminyl groups and galactose groups;
f. cleaving the modified keratan sulfate into a plurality of disaccharide component parts;
g. measuring:
   i. the amount of keratan sulfate disaccharides produced by the second mammalian cell,
   ii. the amount of 6-OH sulfation of the glucosaminyl groups, the 6-OH sulfation of the galactose groups, or a combination thereof of the modified keratan sulfate,
   iii. the pattern of sulfation (domain organization); or
   iv. a combination thereof; and
h. comparing:
   i. the amounts of keratan sulfate disaccharides produced by the first and second mammalian cells,
   ii. the amounts of 6-OH sulfation of the glucosaminyl groups, the 6-OH sulfation of the galactose groups, pattern of sulfation, or a combination thereof of the keratan sulfate and the modified keratan sulfate, or
   iii. a combination thereof.

In some embodiments, the mammalian cell (e.g., human cell) is selected from any suitable mammalian cell. In specific embodiments, the mammalian cell is, by way of non-limiting example, a human cancer cell (e.g., human cervical cancer cell (HeLa)) a human ovarian cancer cell (SKOV), a human lung cancer cell (Hal8), a human meduloblastoma cancer cell (DAOY), a human melanoma cell (SK-MEL), or a human primary cell. Furthermore, in some embodiments, the process is repeated utilizing one or more additional cell types. In certain embodiments, the results (e.g., of (c), (g), and/or (h)) from the one or more additional cell types (e.g., a second, third, fourth, fifth or the like cell types) are compared to each other and the results (e.g., of (c), (g), and/or (h)) from the first cell type.

In certain embodiments, the chondroitin sulfate and/or the modified chondroitin sulfate are cleaved in any suitable manner. In some embodiments, the chondroitin sulfate and/or the modified chondroitin sulfate are cleaved using a suitable enzyme such as chondroitinase AC from Pedobacter heparinus or Arthrobacter aurescens or chondroitinase ABC from Proteus vulgaris, or in any other suitable chemical manner.

In some embodiments, the amount of disaccharide units present in the cell and/or the characteristic of the sulfation in a cell are determined in any suitable manner. For example, in some embodiments, the amount of disaccharide present and/or the amount of 6-OH sulfation of the galactosaminyl groups, the 4-OH sulfation of the galactosaminyl groups, the 2-OH sulfation of the uronic acid groups, or a combination thereof is determined utilizing a carbozole assay, high performance liquid chromatography (HPLC), capillary elecrophoresis, gel electrophoseis, mass spectrum (MS) analysis, nuclear magnetic resonance (NMR) analysis, or the like.

Moreover, in certain embodiments, the process described is a process for identifying compounds that selectively modulate chondroitin sulfate biosynthesis. In such embodiments, the process also comprises collecting one or more non-chondroitin sulfate glycan (e.g., a sulfated glycan, such as chondroitin sulfate, O-linked glycans, N-linked glycans, gangliosides, or the like) from the cell, both without incubation with the compound and with incubation with the compound; cleaving each of such non-chondroitin sulfate glycans; measuring the character of each of such non-chondroitin sulfate glycan; and comparing the character of the non-chondroitin sulfate glycan that was not incubated with the character of the non-chondroitin sulfate glycan that was incubated. In certain embodiments, the character includes, by way of non-limiting example, the chain length of the non-chondroitin sulfate glycan, the amount of sulfation of the non-chondroitin sulfate glycan, the location of sulfation of the non-chondroitin sulfate glycan, the structure of the non-chondroitin sulfate glycan , the composition of the non-chondroitin sulfate glycan, or the like. The structure of glycosaminoglycans, N-linked glycans, O-linked glycans, and lipid linked glycans can be determined using any suitable method, including, by way of non-limiting example, monosaccharide compositional analysis, capillary electrophoresis, gel electrophoresis, gel filtration, high performance liquid chromatography (HPLC), thin layer chromatography (TLC), mass spectrum (MS) analysis, nuclear magnetic resonance (NMR) analysis, or the like.

### Combinations

In certain instances, it is appropriate to administer at least one therapeutic compound described herein (e.g., any glycan inhibitor described herein) in combination with another therapeutic agent. By way of example only, if one of the side effects experienced by a patient upon receiving one of the glycan inhibitors described herein is nausea, then it is appropriate in certain instances to administer an anti-nausea agent in combination with the initial therapeutic agent. Or, by way of example only, the therapeutic effectiveness of one of the glycan inhibitors described herein is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by a patient is increased by administering one of glycan inhibitors described herein with another therapeutic agent (which also includes a therapeutic regimen) that also has therapeutic benefit. In any case, regardless of the disease, disorder or condition being treated, the overall benefit experienced by the patient is in some embodiments additive of the two therapeutic agents or in other embodiments, the patient experiences a synergistic benefit.

In some embodiments, the particular choice of compounds depends upon the diagnosis of the attending physicians and their judgment of the condition of the patient and the appropriate treatment protocol. The compounds are optionally administered concurrently (e.g., simultaneously, essentially simultaneously or within the same treatment protocol) or sequentially, depending upon the nature of the disease, disorder, or condition, the condition of the patient, and the actual choice of compounds used. In certain instances, the determination of the order of administration, and the number of repetitions of administration of each therapeutic agent during a treatment protocol, is based on an evaluation of the disease being treated and the condition of the patient.

In some embodiments, therapeutically-effective dosages vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a glycan inhibitor described herein) are optionally administered in any order or even simultaneously. If simultaneously, the multiple therapeutic agents are optionally provided in a single, unified form, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing, e.g., from more than zero weeks to less than four weeks. In some embodiments, the additional therapeutic agent is utilized to achieve remission (partial or complete) of a cancer, whereupon the therapeutic agent described herein (e.g., any glycan inhibitor identified according to a process described herein) is subsequently administered. In addition, the combination methods, compositions and formulations are not to be limited to the use of only two agents; the use of multiple therapeutic combinations is also envisioned (including two or more therapeutic compounds described herein).

In certain embodiments, a dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in various embodiments, the dosage regimen actually employed varies and deviates from the dosage regimens set forth herein.

In some embodiments, the pharmaceutical agents which make up the combination therapy disclosed herein are provided in a combined dosage form or in separate dosage forms intended for substantially simultaneous administration. In certain embodiments, the pharmaceutical agents that make up the combination therapy are administered sequentially, with either therapeutic compound being administered by a regimen calling for two-step administration. In some embodiments, two-step administration regimen calls for sequential administration of the active agents or spaced-apart administration of the separate active agents. In certain embodiments, the time period between the multiple administration steps varies, by way of non-limiting example, from a few minutes to several hours, depending upon the properties of each pharmaceutical agent, such as potency, solubility, bioavailability, plasma half-life and kinetic profile of the pharmaceutical agent.

In addition, the chondroitin sulfate inhibitors described herein also are optionally used in combination with procedures that provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a gene or gene mutation that is known to be correlated with certain diseases or conditions.

In various embodiments, the chondroitin sulfate inhibitors described herein and combination therapies are administered before, during or after the occurrence of a disease or condition. Timing of administering the composition containing a chondroitin sulfate inhibitor is optionally varied to suit the needs of the individual treated. Thus, in certain embodiments, the chondroitin sulfate inhibitors are used as a prophylactic and are administered continuously to subjects with a propensity to develop conditions or diseases in order to prevent the occurrence of the disease or condition. In some embodiments, the compounds and compositions are administered to a subject during or as soon as possible after the onset of the symptoms. The administration of the chondroitin sulfate inhibitors are optionally initiated within the first 48 hours of the onset of the symptoms, within the first 6 hours of the onset of the symptoms, or within 3 hours of the onset of the symptoms. The initial administration is achieved by any route practical, such as, for example, an intravenous injection, a bolus injection, infusion over 5 minutes to about 5 hours, a pill, a capsule, transdermal patch, buccal delivery, and the like, or combination thereof. In some embodiments, the compound should be administered as soon as is practicable after the onset of a disease or condition is detected or suspected, and for a length of time necessary for the treatment of the disease, such as, for example, from about 1 month to about 3 months. The length of treatment is optionally varied for each subject based on known criteria. In exemplary embodiments, the compound or a formulation containing the compound is administered for at least 2 weeks, between about 1 month to about 5 years, or from about 1 month to about 3 years.

In certain embodiments, therapeutic agents are combined with or utilized in combination with one or more of the following therapeutic agents in any combination: immunosuppressants or anti-cancer therapies (e.g., radiation, surgery or anti-cancer agents).

In some embodiments, one or more of the anti-cancer agents are proapoptotic agents. Examples of anti-cancer agents include, by way of non-limiting example: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

Other anti-cancer agents include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rlL2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, ete.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents include, but are not limited to, nitrogen mustards (e.g., mechloroethamine, cyclophosphamide, chlorambucil, meiphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, ete.). Examples of antimetabolites include, but are not limited to folic acid analog (e.g., methotrexate), or pyrimidine analogs (e.g., fluorouracil, floxouridine, Cytarabine), purine analogs (e.g., mercaptopurine, thioguanine, pentostatin.

Examples of hormones and antagonists include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

In some embodiments, provided herein is a method of treating lymphoma comprising administering a therapeutically effective amount of a compound described herein in combination with an antibody to CD20 and/or a CHOP (cyclophosphamide, doxorubicin, vincristine, and prednisone) therapy. In certain embodiments, provided herein is a method of treating leukemia comprising administering a therapeutically effective amount of a compound described herein in combination with ATRA, methotrexate, cyclophosphamide and the like.

### Pharmaceutical Compositions

In certain embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition, as used herein, refers to a mixture of a glycan inhibitor described herein with other chemical components, such as carriers, stabilizers, diluents, dispersing agents, suspending agents, thickening agents, and/or excipients. In certain instances, the pharmaceutical composition facilitates administration of the glycan inhibitor to an individual or cell. In certain embodiments of practicing the methods of treatment or use provided herein, therapeutically effective amounts of glycan inhibitors described herein are administered in a pharmaceutical composition to an individual having a disease, disorder, or condition to be treated. In specific embodiments, the individual is a human. As discussed herein, the glycan inhibitors described herein are either utilized singly or in combination with one or more additional therapeutic agents.

In certain embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein are optionally manufactured in a conventional manner, such as, by way of example only, by means of conventional mixing, dissolving, granulating, dragee-making, levigating, emulsifying, encapsulating, entrapping or compression processes.

In certain embodiments, a pharmaceutical compositions described herein includes one or more glycan inhibitor described herein as an active ingredient in free-acid or free-base form, or in a pharmaceutically acceptable salt form. In some embodiments, the compounds described herein are utilized as an N-oxide or in a crystalline or amorphous form (i.e., a polymorph). In certain embodiments, an active metabolite or prodrug of a compound described herein is utilized. In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the glycan inhibitors presented herein are also considered to be disclosed herein.

A “carrier” includes, in some embodiments, a pharmaceutically acceptable excipient and is selected on the basis of compatibility with glycan inhibitors disclosed herein and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

Moreover, in certain embodiments, the pharmaceutical compositions described herein is formulated as a dosage form. As such, in some embodiments, provided herein is a dosage form comprising a glycan inhibitor described herein suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In certain embodiments, pharmaceutical compositions described herein are in or are used in any suitable form. Non-limiting examples of suitable dosage form for various embodiments described herein include dosage forms suitable for oral administration, nasal administration, pulmonary administration, ocular administration, systemic delivery, topical delivery or administration, intrathecal administration, intraperitoneal administration, intravenous administration, intraarterial administration, intracardiac administration, intraosseous administration, intraarticular administration, intrasynovial administration, intracutaneous administration, subcutaneous administration, intramuscular administration, and intradermal administration, intracranial administration, intralesional administration, or intratumoral administration.

The pharmaceutical solid dosage forms described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the glycan inhibitor. In one embodiment, a glycan inhibitor described herein is in the form of a particle and some or all of the particles of the compound are coated. In certain embodiments, some or all of the particles of a glycan inhibitor described herein are microencapsulated. In some embodiment, the particles of the glycan inhibitor described herein are not microencapsulated and are uncoated.

In certain embodiments, the pharmaceutical composition described herein is in unit dosage forms suitable for single administration of precise dosages. In unit dosage form, the formulation is divided into unit doses containing appropriate quantities of one or more therapeutic compound. In some embodiments, the unit dosage is in the form of a package containing discrete quantities of the formulation. Non-limiting examples are packaged tablets or capsules, and powders in vials or ampoules. Aqueous suspension compositions are optionally packaged in single-dose non-reclosable containers. In some embodiments, multiple-dose re-closeable containers are used. In certain instances, multiple dose containers comprise a preservative in the composition. By way of example only, formulations for parenteral injection are presented in unit dosage form, which include, but are not limited to ampoules, or in multi-dose containers, with an added preservative.

### EXAMPLES

### Example 1

### Neurite Outgrowth in Neurons

Cell Culture.

Cultures of cerebellar granule neurons [CGN] are prepared by removing cerebella from postnatal 5-8 d C56BL/6 mice and collected in Hank's Balanced Salt Solution. The cerebella are dissociated by trypsin and DNase, strained through a 40 μm cell strainer and resuspended in Neurobasal-A media supplemented with B27, 2 mM glutamine, 25 mM KCl, and 1% P/S antibiotics [CGN medium]. Neu7cells, an astrocytic cell line inhibitory to neurite outgrowth by producing glycan extracellular matrices, are grown in DMEM containing 10% fetal bovine serum and 1% P/S (see Fok-Seang J et al.: An analysis of astrocytic cell lines with different abilities to promote axon growth; Brain Res. 1995, 689:207-223.)

Experimental Procedure.

Conditioned medium is collected from control Neu7 cells or Neu7 cells treated with a glycan inhibitor described herein after 72 h of plating and mixed with laminin in DMEM and immobolized on coverslips for 4 h at 37° C. The condition medium is removed and CGNs are cultured on the coverslips for 48 h in CGN medium. After 48 h, CGNs are fixed and stained for tubulin. Images are obtained from randomly selected fields and analyzed for neurite length using ImageJ from cells incubated with conditioned medium from Neu7 cells alone or Neu7 cells treated with any glycan inhibitor described herein.

### Example 2

### Animal Model of Axon Regeneration

Experimental Procedure.

A rodent laceration model of spinal cord injury is used on Sprague-Dawley rats (see Chen J, Xu Z C, Xu X-M, Zhang J H: Animal Models of Acute Neurological Injuries, 2009). The rats are separated into two groups, where one group receives a daily intrathecal administration of any glycan inhibitor described herein while the other group receives a vehicle control for 6 weeks. After 6 weeks, animals are sacrificed and axon regeneration is measure by the length of axon growth across the laceration gap and into the distal spinal cord stump. Sensory axonal regeneration is traced via Cholera toxin subunit B (CTB) injection into the sciatic, median, and ulnar nerves. Chondroitin sulfate proteoglycan levels are measured using CS-56 expression, a marker of astrocytic differentiation. Chondroitin sulfate proteoglycan breakdown products are measured by 2-B-6 expression.

### Example 3

### Treatment for Acute Spinal Cord Injury

Human Clinical Trial of the Safety and/or Efficacy of Glycan Inhibitor for Acute Spinal Cord Injury Therapy.

Objective: To determine the safety, tolerability, feasibility, pharmacokinetics and efficacy of intrathecally-administered composition comprising any glycan biosynthesis modulator described herein (e.g., a chondroitin biosynthesis inhibitor) in acute spinal cord injury patients.

Study Design: This study is a Phase I, single-center, open-label, non-randomized dose escalation study followed by a Phase II study in acute spinal cord injury patients. Patients must not have received treatment for their spinal cord injury within 2 weeks of beginning the trial. Treatments include the use of stem cell therapy, pharmaceutical therapy, and biologic therapy such as monoclonal antibodies. Patients must have recovered from all toxicities (to grade 0 or 1) associated with previous treatment. Patients' injury is classified as American Spinal Injury Association Impairment Scale A with clinical evidence of lesions located below c-spine 5 (C-5). Tetraplegic patients who were initially diagnosed as ASIA A (neurologically complete lesion) at screening and turned into ASIA B (neurologically incomplete lesion) at baseline are accepted. Spinal cord injuries must have occurred within 60 months of beginning the trial. The time between the injury and enrollment must be greater than 2 weeks. All subjects are evaluated for safety and all cerebrospinal fluid collections for pharmacokinetic analysis are collected as scheduled. All studies are performed with institutional ethics committee approval and patient consent.

Phase I:

The starting dose for this trial is derived from pharmacokinetic simulations that utilized data from prior studies of intrathecal glycan biosynthesis modulator. The simulations are performed to estimate the length of time that ventricular CSF concentrations of glycan biosynthesis modulator would remain above an optimal “target level”. Dose escalations for patient cohorts are conducted following the traditional phase 1 design in order to determine the maximum tolerated dose (MTD). The MTD is called pharmacokinetically optimal if that dose achieves the targeted PK parameter in at least 90% of the patients treated at that dose level.

Phase II:

Patients receive a continuous intrathecal infusion (lumbar puncture) of glycan inhibitor at every treatment visit. Assessment of CSF pharmacokinetic occurs at predefined visits.

The study involves a pretreatment baseline series of visits, followed by a 2-year treatment period. Participants provide cerebrospinal fluid throughout treatment as directed by the study researchers, and additional studies may be performed during the study period if participants consent to further investigation.

Baseline Visits:

Visit 1: Participants provide cerebrospinal fluid and have a magnetic resonance imaging (MRI) scan of the brain.

Visits 2 and 3: Participants have an MRI scan of the spine, additional electrophysiology tests, and a lumbar puncture to collect a sample of cerebrospinal fluid.

Treatment Visits:

Visit 4: Participants are admitted for a 2-day inpatient stay, and have MRI scans, electrophysiology tests, and provide cerebrospinal fluid on the first day. On the second day, participants receive glycan biosynthesis modulator by intrathecal infusion (lumbar puncture) and are discharged on the following day after overnight monitoring.

Visit 5: Two weeks after Visit 4, participants have an overnight stay to receive any glycan biosynthesis modulator by intrathecal infusion (lumbar puncture).

Visit 6: Six months after Visit 5, participants have MRI scans, electrophysiology tests, and provide cerebrospinal fluid.

Visit 7: One year after Visit 4, participants have another 2-day inpatient stay. On the first day, the same procedures performed described for Visit 4 are repeated; on the second day, participants receive glycan biosynthesis modulator through a intrathecal infusion (lumbar puncture), and are discharged on the following day after overnight monitoring.

Visit 8: Six months after Visit 7, participants have MRI scans, perform electrophysiology tests, and provide cerebrospinal fluid.

Visit 9: Six months after Visit 8, participants have MRI scans, perform electrophysiology tests, and provide cerebrospinal fluid.

After the end of the study, participants continue with standard care for acute spinal cord injury.

Safety is evaluated by neurological and non-neurological tests performed after short-term (1 to 30 days) and long-term (2 to 12 months) follow-up evaluation periods after cell infusion. Early potential signal of efficacy is assessed by the American Spinal Cord Injury Association (ASIA) protocol and pharmacodynamic changes are assessed by electrophysiology tests for up to 2 years.

Pharmacokinetics: Patients undergo cerebrospinal fluid sample collection for pharmacokinetic evaluation. Pharmacokinetic parameters are calculated by model independent methods on a Digital Equipment Corporation VAX 8600 computer system using the latest version of the BIOAVL software. The following pharmacokinetics parameters are determined: peak concentration (Cₘₐₓ); time to peak concentration (tₘₐₓ); area under the concentration-time curve (AUC) from time zero to the last cerebrospinal fluid sampling time (AUC₀₋₇₂) calculated with the use of the linear trapezoidal rule; and terminal elimination half-life (t_{1/2}), computed from the elimination rₐₜₑ constant. The elimination rate constant is estimated by linear regression of consecutive data point_{s in} the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

### Example 4

### In Vitro Cell Migration and Spreading Assay

Cell Lines.

Two mouse Lewis lung carcinoma (3LL)-derived cell lines with different metastatic potentials are used. Highly metastatic LM66-H11 cells and low metastatic P29 cells are maintained in DMEM supplemented with 10% fetal calf serum and 1% penicillin/streptomycin.

Experimental Procedure.

For the migration assay, filters of 8 μm pores are coated with 30 μg of Matrigel on the upper surface and 0.5m fibronectin on the lower surface of Transwell cell culture chambers. Hepatocyte growth factor (HGF) is used as a chemoattractant in the lower chamber. LM66-H11 and P29 cells are pretreated with increasing concentrations of any glycan inhibitor described herein for 6 to 12 h or left untreated as control. 2×10⁵ cells are added to the upper chamber and incubated for 6 h at 37° C. Filters are then extracted from the lower chamber and cell migration/invasion is assessed by absorbance of cell lysate at 590 nm. In the cell spreading assay, 96 well plates are coated with fibronectin (0.5 m/well) for 24 h and subsequently blocked with 1% BSA. Vehicle treated or any glycan inhibitor described herein treated LM66-H11 and P29 cells are incubated with 30 ng/ml HGF in the fibronectin-coated wells. The cells are then fixed, stained with Giemsa's solution, and observed under a microscope for spreading.

### Example 5

### Chondroitin Sulfate Inhibition

Chondroitin sulfate inhibition was tested with monoclonal antibody 2B6 specific to chondroitinase-generated C-4-S. Monoclonal antibody 2B6 binds to delta-unsaturated glucuronic acid adjacent to N-acetylgalactosamine-4-sulphate in the non-reducing terminal disaccharide “stub” of 4-sulphated chondroitin sulphate that is produced after chondroitinase digestion of chondroitin-4-sulphate glycosaminoglycan chains (chondroitinase ABC or ACII) or dermatan sulphate glycosaminoglycan chains (chondroitinase ABC or chondroitinase B). For these analyses, cells grown in the presence and absence of a test compound for 2 days were released with 5 mM EDTA, neutralized with serum supplemented cell culture medium and separated by centrifugation. The cells were then resuspended in buffered solution and digested with chondroitinase ABC (˜1×10⁻³ U per 10 mg GAG for 20-30 minutes at 37° C.). After digestion the cells were separated by centrifugation and resuspended in 2B6 antibody solution in serum supplemented cell culture medium at various dilutions (e.g. 1:500) and incubated for 1 hour on ice. Following the incubation the cells were rinsed in serum supplemented medium and resuspended in a secondary probe antibody and incubated for 30 minutes on ice. After washing to remove unbound secondary probe antibody the bound probe was quantified using flow cytometry.

### Example 6

### Specificity of Compound Inhibition

Cells treated with the test compound were tested with a panel of lectins to measure the specificity of the test compounds for inhibiting only chondroitin sulfate biosynthesis. The lectin panel shows the effects of the compounds on additional glycans as indicated below. HeLa cells and/or Chinese Hamster Ovary (CHO) cells were treated with and without the test compound. After 2 days of growth, the cells were released with 5 mM EDTA. Parallel cultures were then probed for 1 hour on ice with biotinylated preparations of each of the lectins at various dilutions (e.g. 1:100) in the panel. After washing to remove unbound lectin the lectins were detected with streptavidin-Cy5-PE. After washing to remove the unbound streptavidin-Cy5-PE the bound probes were quantified using flow cytometry.

### Lectin Panel

concanavalin A (ConA) binds α-mannose/α-glucose on simple and biantennary N-glycans
wheat germ-agglutinin (WGA) binds to terminal N-acetylglucosamine on O and N-linked glycans and sialic acid
L-phytohemagglutinin (PHA) binds tri and tetra antennary N-linked glycans
jacalin (JAC) binds O-linked glycans, primarily galactose (β-1,3) N-acetyglucosamine (T-antigen)
Maackia Amurensis lectin II (MAL) binds (α-2,3) linked sialic acid.

### Example 7

### Method of Treatment

Human Clinical Trial of the Safety and/or Efficacy of any glycan inhibitor described herein (or a pharmaceutically acceptable salt thereof) therapy

Objective:

To determine the safety and pharmacokinetics of administered [chondroitin sulfate modulator].

Study Design:

This will be a Phase I, single-center, open-label, randomized dose escalation study followed by a Phase II study in cancer patients with a cancer that can be biopsied (e.g., prostate cancer, pancreatic cancer, colorectal cancer, lung cancer, or ovarian cancer). Patients should not have had exposure to any glycan inhibitor described herein prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I:

Patients receive intravenous any glycan inhibitor described herein daily for 5 consecutive days or 7 days a week. Doses of [chondroitin sulfate modulator] may be held or modified for toxicity based on assessments as outlined below. Treatment repeats every 28 days in the absence of unacceptable toxicity. Cohorts of 3-6 patients receive escalating doses of any glycan inhibitor described herein until the maximum tolerated dose (MTD) for the any glycan inhibitor described herein is determined. The MTD is defined as the dose preceding that at which 2 of 3 or 2 of 6 patients experience dose-limiting toxicity. Dose limiting toxicities are determined according to the definitions and standards set by the National Cancer Institute (NCl) Common Terminology for Adverse Events (CTCAE) Version 3.0 (Aug. 9, 2006).

Phase II:

Patients receive any glycan inhibitor described herein as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling

Serial blood is drawn by direct vein puncture before and after administration of [chondroitin sulfate modulator]. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics:

Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy:

Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH or TaqMan PCR for specific chromosomal translocations. After completion of study treatment, patients are followed periodically for 4 weeks.

What is claimed is:

1. A process for modifying the structure of chondroitin sulfate on a core protein, comprising contacting a cell that translationally produces at least one core protein having at least one attached chondroitin sulfate moiety with a selective inhibitor of a chondroitin sulfate glycosyltransferase, a chondroitin sulfate sulfotransferase, or a chondroitin sulfate phosphotransferase.

2. The process of claim 1, wherein the selective inhibitor of a chondroitin sulfate sulfotransferase is an inhibitor of a chondroitin sulfate O-sulfotransferase.

3. The process of claim 2, wherein the inhibitor of a chondroitin sulfate O-sulfotransferase inhibits the 6-OH sulfation of a galactosaminyl moiety, the 4-OH sulfation of a galactosaminyl moiety, the 2-OH sulfation of a uronic acid moiety, or a combination thereof.

4. The process of claim 1, wherein the inhibitor of a chondroitin sulfate glycosyltransferase inhibits the synthesis of the linkage region, the modification of the linkage region, the initiation of chondroitin sulfate synthesis, the synthesis of chondroitin sulfate, or a combination thereof.

5. A process of inhibiting chondroitin sulfate function in a cell comprising contacting the cell with a selective modulator of a chondroitin sulfate glycosyltransferase or a modulator of a chondroitin sulfate sulfotransferase.

6. The process of claim 5, wherein the chondroitin sulfate function inhibited is an ability to bind a chondroitin sulfate binding lectin.

7. The process of claim 6, wherein the chondroitin sulfate lectin is a growth factor.

8. The process of claim 7, wherein the growth factor is a fibroblast growth factor (FGF), heparin binding epidermal growth factor (HB-EGF), vascular endothelial growth factor (VEGF), pleiotrophin, hepatocyte growth factor, heparin co-factor II or midkine, lamin, nuclear ribonucleoprotein, an antibody, Plasmodium falciparum lectin, annexin 4, annexin 6, PTPsigma, or endostatin.

9. The process of claim 5, wherein the modulator of chondroitin sulfate sulfotransferase is an inhibitor of chondroitin sulfate sulfotransferase.

10. The process of claim 9, wherein the inhibitor of chondroitin sulfate sulfotransferase is an inhibitor of chondroitin sulfate O-sulfotransferase.

11. The process of claim 10, wherein the inhibitor of chondroitin sulfate O-sulfotransferase inhibits the 6-OH sulfation of a galactosaminyl moiety, the 4-OH sulfation of a galactosaminyl moiety, the 2-OH sulfation of a uronic acid moiety, the 6-O sulfation of a galactosyl moiety, the 4-O sulfation of a galactosyl moiety, or a combination thereof.

12. The process of claim 5, wherein the modulator of a chondroitin sulfate sulfotransferase is a promoter of the chondroitin sulfate sulfotransferase.

13. The process of claim 5, wherein the modulator of a chondroitin sulfate glycosyltransferase is an inhibitor of the chondroitin sulfate glycosyltransferase.

14. The process of claim 5, wherein the modulator of a chondroitin sulfate glycosyltransferase is a promoter of the chondroitin sulfate glycosyltransferase.

15. The process of claim 5, wherein the cell is present in a human diagnosed with cancer.

16. A process of inhibiting chondroitin sulfate function in a cell comprising contacting the cell with a selective modulator of chondroitin sulfate biosynthesis.

17. The process of claim 16, wherein the selective modulator of chondroitin sulfate biosynthesis inhibits chondroitin glycosylation.

18. The process of claim 16, wherein the selective modulator of chondroitin sulfate biosynthesis inhibits sulfation of chondroitin.

19. The process of claim 16, wherein the selective modulator of chondroitin sulfate biosynthesis promotes sulfation of chondroitin.

20. The process of any of claims 16-19, wherein the selective modulator of chondroitin sulfate biosynthesis has a molecular weight of less than 1,000 g/mol.

21. A method of treating cancer comprising administering a therapeutically effective amount of a selective modulator of chondroitin sulfate glycosylation, or a selective modulator of chondroitin sulfate sulfation.

22. The method of claim 21, wherein the selective modulator of chondroitin sulfate biosynthesis inhibits chondroitin glycosylation.

23. The method of claim 21, wherein the selective modulator of chondroitin sulfate promotes chondroitin glycosylation.

24. The method of claim 21, wherein the selective modulator of chondroitin sulfate inhibits sulfation of chondroitin.

25. The method of claim 21, wherein the selective modulator of chondroitin sulfate promotes sulfation of chondroitin.

26. The process of any of claims 21-25, wherein the selective modulator of chondroitin sulfate biosynthesis has a molecular weight of less than 1,000 g/mol.

27. A method of treating a lysosomal storage disease comprising administering a therapeutically effective amount of a selective modulator of chondroitin sulfate glycosylation, or a selective modulator of chondroitin sulfate sulfation.

28. The method of claim 27, wherein the lysosomal storage disease is selected from mucopolysaccharidosis.

29. The method of either of claim 27 or 28, wherein the selective modulator of chondroitin sulfate glycosylation is an inhibitor of chondroitin sulfate glycosylation.

30. The method of either of claim 27 or 28, wherein the selective modulator of chondroitin sulfate sulfation is an inhibitor of chondroitin sulfate sulfation.

31. A method of treating an inflammatory disease comprising administering a therapeutically effective amount of a selective modulator of chondroitin sulfate glycosylation, or a selective modulator of chondroitin sulfate sulfation.

32. The method of claim 31, wherein, the inflammatory disease is selected from osteoarthritis.

33. The method of either of claim 31 or 32, wherein the selective modulator of chondroitin sulfate glycosylation is an inhibitor of chondroitin sulfate glycosylation.

34. The method of either of claim 31 or 32, wherein the selective modulator of chondroitin sulfate glycosylation is a promoter of chondroitin sulfate glycosylation.

35. The method of either of claim 31 or 32, wherein the selective modulator of chondroitin sulfate sulfation is an inhibitor of chondroitin sulfate sulfation.

36. The method of either of claim 31 or 32, wherein the selective modulator of chondroitin sulfate sulfation is a promoter of chondroitin sulfate sulfation.

37. The method of any of claims 31-36, wherein the selective modulator of chondroitin sulfate biosynthesis has a molecular weight of less than 1,000 g/mol.

38. A method of treating injury to the central nervous system (CNS) comprising administering a therapeutically effective amount of a selective modulator of chondroitin sulfate glycosylation, or a selective modulator of chondroitin sulfate sulfation, wherein the modulator promotes axon regeneration.

39. The method of claim 38, wherein the selective modulator of chondroitin sulfate glycosylation is an inhibitor of chondroitin sulfate glycosylation.

40. The method of claim 38, wherein the selective modulator of chondroitin sulfate sulfation is an inhibitor of chondroitin sulfate sulfation.

41. A process for identifying a compound that selectively modulates chondroitin sulfate biosynthesis comprising:
a. contacting a mammalian cell with the compound
b. contacting the mammalian cell and compound combination with a first labeled probe and a second labeled probe, wherein the first labeled probe binds chondroitin sulfate and the second labeled probe binds at least one glycan other than chondroitin sulfate;
c. incubating the mammalian cell, compound, the first labeled probe, and the second labeled probe;
d. collecting the first labeled probe that is bound to chondroitin sulfate;
e. collecting the second labeled probe that is bound to at least one glycan other than chondroitin sulfate;
f. detecting or measuring the amount of first labeled probe bound to chondroitin sulfate; and
g. detecting or measuring the amount of the second labeled probe bound to at least one glycan other than chondroitin sulfate.

42. The process of claim 41, wherein the mammalian cell is a human cervical cancer cell (HeLa).

43. The process of claim 41, wherein the labeled probe comprises a biotinyl moiety and the process further comprises tagging the labeled probe with streptavidin-Cy5-PE.

44. The process of claim 41, wherein the labeled probe comprises a fluorescent label.

45. The process of claim 41, wherein labeled probe is a labeled growth factor.

46. The process of claim 45, wherein the labeled growth factor is labeled fibroblast growth factor (FGF).

47. A process for identifying a compound that modulates chondroitin sulfate biosynthesis comprising:
a. collecting chondroitin sulfate from a first mammalian cell of a selected type, wherein the chondroitin sulfate is sulfated oligosaccharide comprising galactosaminyl groups, and glucuronic acid groups;
b. cleaving the chondroitin sulfate into a plurality of disaccharide component parts;
c. measuring:
i. the amount of chondroitin sulfate disaccharides produced by the first mammalian cell,
ii. the amount of 6-OH sulfation of the galactosaminyl groups, the 4-OH sulfation of the galactosaminyl groups, the 2-OH sulfation of the uronic acid groups, or a combination thereof of the chondroitin sulfate,
iii. the pattern of sulfation; or
iv. a combination thereof; and
d. contacting and incubating a second mammalian cell of the selected type with the compound;
e. collecting modified chondroitin sulfate from the second mammalian cell, wherein the modified chondroitin sulfate is sulfated oligosaccharide comprising galactosaminyl groups, and glucuronic acid groups;
f. cleaving the modified chondroitin sulfate into a plurality of disaccharide component parts;
g. measuring:
i. the amount of chondroitin sulfate disaccharides produced by the second mammalian cell,
ii. the amount of 6-OH sulfation of the galactosaminyl groups, the 4-OH sulfation of the galactosaminyl groups, the 2-OH sulfation of the uronic acid groups, or a combination thereof of the modified chondroitin sulfate,
iii. the pattern of sulfation; or
iv. a combination thereof; and
h. comparing:
i. the amounts of chondroitin sulfate disaccharides produced by the first and second mammalian cells,
ii. the amounts of 6-OH sulfation of the galactosaminyl groups, the 4-OH sulfation of the galactosaminyl groups, the 2-OH sulfation of the uronic acid groups, pattern of sulfation, or a combination thereof of the chondroitin sulfate and the modified chondroitin sulfate,
iii. the pattern of sulfation; or
iv. a combination thereof.

48. A process for modifying the structure of chondroitin sulfate on a core protein, comprising contacting a cell that translationally produces at least one core protein having at least one attached chondroitin sulfate moiety with a selective inhibitor of a chondroitin sulfate glycosyltransferase, a chondroitin sulfate sulfotransferase, or a chondroitin sulfate phosphotransferase.

49. A process for modifying the structure of keratan sulfate on a core protein, comprising contacting a cell that translationally produces at least one core protein having at least one attached keratan sulfate moiety with a selective inhibitor of a keratan sulfate glycosyltransferase, a keratan sulfate sulfotransferase, or a keratan sulfate phosphotransferase.

50. A process for modifying the structure of dermatan sulfate on a core protein, comprising contacting a cell that translationally produces at least one core protein having at least one attached dermatan sulfate moiety with a selective inhibitor of a dermatan sulfate glycosyltransferase, a dermatan sulfate sulfotransferase, or a dermatan sulfate phosphotransferase.

51. A process of modulating chondroitin sulfate biosynthesis in a cell comprising contacting the cell with a selective modulator of chondroitin sulfate biosynthesis, wherein the selective modulator is optionally (i) cellularly active, (ii) a non-carbohydrate small molecule having a molecular weight of less than 1000 g/mol, (iii) an inhibitor of chondroitin sulfate biosynthesis, and/or (iv) an indirect modulator (e.g., inhibitor) of chondroitin sulfate biosynthesis.

52. The process of claim 51, wherein the selective modulator of chondroitin sulfate biosynthesis alters or disrupts the chain length of chondroitin sulfate compared to endogenous chondroitin sulfate by at least 5%.

53. The process of claim 51, wherein the selective modulator of chondroitin sulfate biosynthesis alters or disrupts the concentration of chondroitin sulfate compared to endogenous chondroitin sulfate by at least 5%.

54. The process of claim 51, wherein the selective modulator of chondroitin sulfate biosynthesis alters or disrupts the 2-O sulfation of chondroitin sulfate compared to endogenous chondroitin sulfate by at least 5%.

55. The process of claim 51, wherein the selective modulator of chondroitin sulfate biosynthesis alters or disrupts the 4-O sulfation of chondroitin sulfate compared to endogenous chondroitin sulfate by at least 5%.

56. The process of claim 51, wherein the selective modulator of chondroitin sulfate biosynthesis alters or disrupts the 6-O sulfation of chondroitin sulfate compared to endogenous chondroitin sulfate by at least 5%.

57. The process of claim 51, wherein the selective modulator of chondroitin sulfate biosynthesis alters the ratio of 4-O sulfation to 6-O sulfation to below 6.3 to 1.

58. The process of claim 51, wherein the selective modulator of chondroitin sulfate biosynthesis alters the ratio of 6-O sulfation to 2-O sulfation to below 0.16 to 1.

59. The process of claim 51, wherein the selective modulator is a direct modulator (e.g., inhibitor) of chondroitin sulfate biosynthesis

60. A process of modulating dermatan sulfate biosynthesis in a cell comprising contacting the cell with a selective modulator of dermatan sulfate biosynthesis, wherein the selective modulator is optionally (i) cellularly active, (ii) a non-carbohydrate small molecule having a molecular weight of less than 1000 g/mol, (iii) an inhibitor of dermatan sulfate biosynthesis, and/or (iv) an indirect modulator (e.g., inhibitor) of dermatan sulfate biosynthesis.

61. The process of claim 60, wherein the selective modulator of dermatan sulfate biosynthesis alters the ratio of 2-O sulfation to 4-O sulfation to below 0.13 to 1.

62. The process of claim 60, wherein the selective modulator of dermatan sulfate biosynthesis alters the ratio of 2-O sulfation to 6-O sulfation to below 1.44 to 1.

63. The process of claim 60, wherein the selective modulator of dermatan sulfate biosynthesis alters the ratio of 4-O sulfation to 6-O sulfation to below 11.4 to 1.

64. The process of claim 60, wherein the selective modulator of dermatan sulfate biosynthesis alters the ratio of 4-O sulfation to 2-O sulfation to below 7.9 to 1.

65. The process of claim 60, wherein the selective modulator of dermatan sulfate biosynthesis alters the ratio of 6-O sulfation to 2-O sulfation to below 0.7 to 1.

66. The process of claim 60, wherein the selective modulator of dermatan sulfate biosynthesis alters the ratio of 6-O sulfation to 4-O sulfation to below 0.09 to 1.

67. The process of claim 60, wherein the selective modulator is a direct modulator (e.g., inhibitor) of dermatan sulfate biosynthesis
